SEQUENCE LISTING (1001 sequences)

SEQ ID NO: 1 - RNA, 20 nt, synthetic construct
  misc_feature 1..20: note = Synthetic
  source 1..20: mol_type = other RNA
aggcgccctggccagtcgtc

SEQ ID NO: 2 - RNA, 20 nt, synthetic construct
  misc_feature 1..20: note = Synthetic
  source 1..20: mol_type = other RNA
ggcgccctggccagtcgtct

SEQ ID NO: 3 - RNA, 20 nt, synthetic construct
  misc_feature 1..20: note = Synthetic
  source 1..20: mol_type = other RNA
caccgcccagacgactggcc

SEQ ID NO: 4 - RNA, 20 nt, synthetic construct
  misc_feature 1..20: note = Synthetic
  source 1..20: mol_type = other RNA
tgtagcaccgcccagacgac

SEQ ID NO: 5 - RNA, 20 nt, synthetic construct
  misc_feature 1..20: note = Synthetic
  source 1..20: mol_type = other RNA
cgtctgggcggtgctacaac

SEQ ID NO: 6 - RNA, 20 nt, synthetic construct
  misc_feature 1..20: note = Synthetic
  source 1..20: mol_type = other RNA
gtctgggcggtgctacaact

SEQ ID NO: 7 - RNA, 20 nt, synthetic construct
  misc_feature 1..20: note = Synthetic
  source 1..20: mol_type = other RNA
gggcggtgctacaactgggc

SEQ ID NO: 8 - RNA, 20 nt, synthetic construct
  misc_feature 1..20: note = Synthetic
  source 1..20: mol_type = other RNA
cacctacctaagaaccatcc

SEQ ID NO: 9 - RNA, 20 nt, synthetic construct
  misc_feature 1..20: note = Synthetic
  source 1..20: mol_type = other RNA
cggtcaccacgagcagggct

SEQ ID NO: 10 - RNA, 20 nt, synthetic construct
  misc_feature 1..20: note = Synthetic
  source 1..20: mol_type = other RNA
tcggtcaccacgagcagggc

SEQ ID NO: 11 - RNA, 20 nt, synthetic construct
  misc_feature 1..20: note = Synthetic
  source 1..20: mol_type = other RNA
gccctgctcgtggtgaccga

SEQ ID NO: 12 - RNA, 20 nt, synthetic construct
  misc_feature 1..20: note = Synthetic
  source 1..20: mol_type = other RNA
cccttcggtcaccacgagca

SEQ ID NO: 13 - RNA, 20 nt, synthetic construct
  misc_feature 1..20: note = Synthetic
  source 1..20: mol_type = other RNA
ccccttcggtcaccacgagc

SEQ ID NO: 14 - RNA, 20 nt, synthetic construct
  misc_feature 1..20: note = Synthetic
  source 1..20: mol_type = other RNA
cctgctcgtggtgaccgaag

SEQ ID NO: 15 - RNA, 20 nt, synthetic construct
  misc_feature 1..20: note = Synthetic
  source 1..20: mol_type = other RNA
cacgaagctctccgatgtgt

SEQ ID NO: 16 - RNA, 20 nt, synthetic construct
  misc_feature 1..20: note = Synthetic
  source 1..20: mol_type = other RNA
cggagagcttcgtgctaaac

SEQ ID NO: 17 - RNA, 20 nt, synthetic construct
  misc_feature 1..20: note = Synthetic
  source 1..20: mol_type = other RNA
gcttgtccgtctggttgctg

SEQ ID NO: 18 - RNA, 20 nt, synthetic construct
  misc_feature 1..20: note = Synthetic
  source 1..20: mol_type = other RNA
agcttgtccgtctggttgct

SEQ ID NO: 19 - RNA, 20 nt, synthetic construct
  misc_feature 1..20: note = Synthetic
  source 1..20: mol_type = other RNA
cagcttgtccgtctggttgc

SEQ ID NO: 20 - RNA, 20 nt, synthetic construct
  misc_feature 1..20: note = Synthetic
  source 1..20: mol_type = other RNA
aggcggccagcttgtccgtc

SEQ ID NO: 21 - RNA, 20 nt, synthetic construct
  misc_feature 1..20: note = Synthetic
  source 1..20: mol_type = other RNA
tgacacggaagcggcagtcc

SEQ ID NO: 22 - RNA, 20 nt, synthetic construct
  misc_feature 1..20: note = Synthetic
  source 1..20: mol_type = other RNA
gcagttgtgtgacacggaag

SEQ ID NO: 23 - RNA, 20 nt, synthetic construct
  misc_feature 1..20: note = Synthetic
  source 1..20: mol_type = other RNA
cgtgtcacacaactgcccaa

SEQ ID NO: 24 - RNA, 20 nt, synthetic construct
  misc_feature 1..20: note = Synthetic
  source 1..20: mol_type = other RNA
gtgtcacacaactgcccaac

SEQ ID NO: 25 - RNA, 20 nt, synthetic construct
  misc_feature 1..20: note = Synthetic
  source 1..20: mol_type = other RNA
atgtggaagtcacgcccgtt

SEQ ID NO: 26 - RNA, 20 nt, synthetic construct
  misc_feature 1..20: note = Synthetic
  source 1..20: mol_type = other RNA
gcgtgacttccacatgagcg

SEQ ID NO: 27 - RNA, 20 nt, synthetic construct
  misc_feature 1..20: note = Synthetic
  source 1..20: mol_type = other RNA
acttccacatgagcgtggtc

SEQ ID NO: 28 - RNA, 20 nt, synthetic construct
  misc_feature 1..20: note = Synthetic
  source 1..20: mol_type = other RNA
cttccacatgagcgtggtca

SEQ ID NO: 29 - RNA, 20 nt, synthetic construct
  misc_feature 1..20: note = Synthetic
  source 1..20: mol_type = other RNA
agggcccggcgcaatgacag

SEQ ID NO: 30 - RNA, 20 nt, synthetic construct
  misc_feature 1..20: note = Synthetic
  source 1..20: mol_type = other RNA
ggtgccgctgtcattgcgcc

SEQ ID NO: 31 - RNA, 20 nt, synthetic construct
  misc_feature 1..20: note = Synthetic
  source 1..20: mol_type = other RNA
gatctgcgccttgggggcca

SEQ ID NO: 32 - RNA, 20 nt, synthetic construct
  misc_feature 1..20: note = Synthetic
  source 1..20: mol_type = other RNA
ctctctttgatctgcgcctt

SEQ ID NO: 33 - RNA, 20 nt, synthetic construct
  misc_feature 1..20: note = Synthetic
  source 1..20: mol_type = other RNA
gctctctttgatctgcgcct

SEQ ID NO: 34 - RNA, 20 nt, synthetic construct
  misc_feature 1..20: note = Synthetic
  source 1..20: mol_type = other RNA
agggtttggaactggccggc

SEQ ID NO: 35 - RNA, 20 nt, synthetic construct
  misc_feature 1..20: note = Synthetic
  source 1..20: mol_type = other RNA
accctggtggttggtgtcgt

SEQ ID NO: 36 - RNA, 20 nt, synthetic construct
  misc_feature 1..20: note = Synthetic
  source 1..20: mol_type = other RNA
gcccacgacaccaaccacca

SEQ ID NO: 37 - RNA, 20 nt, synthetic construct
  misc_feature 1..20: note = Synthetic
  source 1..20: mol_type = other RNA
cgcccacgacaccaaccacc

SEQ ID NO: 38 - RNA, 20 nt, synthetic construct
  misc_feature 1..20: note = Synthetic
  source 1..20: mol_type = other RNA
ctggtggttggtgtcgtggg

SEQ ID NO: 39 - RNA, 20 nt, synthetic construct
  misc_feature 1..20: note = Synthetic
  source 1..20: mol_type = other RNA
tcctggccgtcatctgctcc

SEQ ID NO: 40 - RNA, 20 nt, synthetic construct
  misc_feature 1..20: note = Synthetic
  source 1..20: mol_type = other RNA
gacgttacctcgtgcggccc

SEQ ID NO: 41 - RNA, 20 nt, synthetic construct
  misc_feature 1..20: note = Synthetic
  source 1..20: mol_type = other RNA
tgacgttacctcgtgcggcc

SEQ ID NO: 42 - RNA, 20 nt, synthetic construct
  misc_feature 1..20: note = Synthetic
  source 1..20: mol_type = other RNA
tgggatgacgttacctcgtg

SEQ ID NO: 43 - RNA, 20 nt, synthetic construct
  misc_feature 1..20: note = Synthetic
  source 1..20: mol_type = other RNA
cgactggccagggcgcctgt

SEQ ID NO: 44 - RNA, 20 nt, synthetic construct
  misc_feature 1..20: note = Synthetic
  source 1..20: mol_type = other RNA
tgcggtaccagtttagcacg

SEQ ID NO: 45 - RNA, 20 nt, synthetic construct
  misc_feature 1..20: note = Synthetic
  source 1..20: mol_type = other RNA
aggtgcccattccgctagga

SEQ ID NO: 46 - RNA, 20 nt, synthetic construct
  misc_feature 1..20: note = Synthetic
  source 1..20: mol_type = other RNA
ggcccccaaggcgcagatca

SEQ ID NO: 47 - RNA, 20 nt, synthetic construct
  misc_feature 1..20: note = Synthetic
  source 1..20: mol_type = other RNA
tggatttccagtggcgagag

SEQ ID NO: 48 - RNA, 20 nt, synthetic construct
  misc_feature 1..20: note = Synthetic
  source 1..20: mol_type = other RNA
acctcgtgcggcccgggagc

SEQ ID NO: 49 - RNA, 20 nt, synthetic construct
  misc_feature 1..20: note = Synthetic
  source 1..20: mol_type = other RNA
atcaaagagagcctgcgggc

SEQ ID NO: 50 - RNA, 20 nt, synthetic construct
  misc_feature 1..20: note = Synthetic
  source 1..20: mol_type = other RNA
ccgccgaccccacctaccta

SEQ ID NO: 51 - RNA, 20 nt, synthetic construct
  misc_feature 1..20: note = Synthetic
  source 1..20: mol_type = other RNA
gtggcatactccgtctgctc

SEQ ID NO: 52 - RNA, 20 nt, synthetic construct
  misc_feature 1..20: note = Synthetic
  source 1..20: mol_type = other RNA
ctacaactgggctggcggcc

SEQ ID NO: 53 - RNA, 20 nt, synthetic construct
  misc_feature 1..20: note = Synthetic
  source 1..20: mol_type = other RNA
tggccaaggaagccggtcag

SEQ ID NO: 54 - RNA, 20 nt, synthetic construct
  misc_feature 1..20: note = Synthetic
  source 1..20: mol_type = other RNA
tgctggggctcatgcggtac

SEQ ID NO: 55 - RNA, 20 nt, synthetic construct
  misc_feature 1..20: note = Synthetic
  source 1..20: mol_type = other RNA
gctacaactgggctggcggc

SEQ ID NO: 56 - RNA, 20 nt, synthetic construct
  misc_feature 1..20: note = Synthetic
  source 1..20: mol_type = other RNA
ccagtggcgagagaagaccc

SEQ ID NO: 57 - RNA, 20 nt, synthetic construct
  misc_feature 1..20: note = Synthetic
  source 1..20: mol_type = other RNA
tgcggcccgggagcagatga

SEQ ID NO: 58 - RNA, 20 nt, synthetic construct
  misc_feature 1..20: note = Synthetic
  source 1..20: mol_type = other RNA
cgttgggcagttgtgtgaca

SEQ ID NO: 59 - RNA, 20 nt, synthetic construct
  misc_feature 1..20: note = Synthetic
  source 1..20: mol_type = other RNA
gccaccattgtctttcctag

SEQ ID NO: 60 - RNA, 20 nt, synthetic construct
  misc_feature 1..20: note = Synthetic
  source 1..20: mol_type = other RNA
cccccaaggcgcagatcaaa

SEQ ID NO: 61 - RNA, 20 nt, synthetic construct
  misc_feature 1..20: note = Synthetic
  source 1..20: mol_type = other RNA
gtgctaaactggtaccgcat

SEQ ID NO: 62 - RNA, 20 nt, synthetic construct
  misc_feature 1..20: note = Synthetic
  source 1..20: mol_type = other RNA
cgaagctctccgatgtgttg

SEQ ID NO: 63 - RNA, 20 nt, synthetic construct
  misc_feature 1..20: note = Synthetic
  source 1..20: mol_type = other RNA
aacgggcgtgacttccacat

SEQ ID NO: 64 - RNA, 20 nt, synthetic construct
  misc_feature 1..20: note = Synthetic
  source 1..20: mol_type = other RNA
ttgtccccttcggtcaccac

SEQ ID NO: 65 - RNA, 20 nt, synthetic construct
  misc_feature 1..20: note = Synthetic
  source 1..20: mol_type = other RNA
gcagcttctccaacacatcg

SEQ ID NO: 66 - RNA, 20 nt, synthetic construct
  misc_feature 1..20: note = Synthetic
  source 1..20: mol_type = other RNA
gtggccaaggaagccggtca

SEQ ID NO: 67 - RNA, 20 nt, synthetic construct
  misc_feature 1..20: note = Synthetic
  source 1..20: mol_type = other RNA
cccaaggcgcagatcaaaga

SEQ ID NO: 68 - RNA, 20 nt, synthetic construct
  misc_feature 1..20: note = Synthetic
  source 1..20: mol_type = other RNA
agcccctgcgggcgggggat

SEQ ID NO: 69 - RNA, 20 nt, synthetic construct
  misc_feature 1..20: note = Synthetic
  source 1..20: mol_type = other RNA
catctgctcccgggccgcac

SEQ ID NO: 70 - RNA, 20 nt, synthetic construct
  misc_feature 1..20: note = Synthetic
  source 1..20: mol_type = other RNA
agcttctccaacacatcgga

SEQ ID NO: 71 - RNA, 20 nt, synthetic construct
  misc_feature 1..20: note = Synthetic
  source 1..20: mol_type = other RNA
agtggcgagagaagaccccg

SEQ ID NO: 72 - RNA, 20 nt, synthetic construct
  misc_feature 1..20: note = Synthetic
  source 1..20: mol_type = other RNA
acaagctggccgccttcccc

SEQ ID NO: 73 - RNA, 20 nt, synthetic construct
  misc_feature 1..20: note = Synthetic
  source 1..20: mol_type = other RNA
cactccgagggccgtcagct

SEQ ID NO: 74 - RNA, 20 nt, synthetic construct
  misc_feature 1..20: note = Synthetic
  source 1..20: mol_type = other RNA
cgttacctcgtgcggcccgg

SEQ ID NO: 75 - RNA, 20 nt, synthetic construct
  misc_feature 1..20: note = Synthetic
  source 1..20: mol_type = other RNA
acgaagctctccgatgtgtt

SEQ ID NO: 76 - RNA, 20 nt, synthetic construct
  misc_feature 1..20: note = Synthetic
  source 1..20: mol_type = other RNA
ctgctcgtggtgaccgaagg

SEQ ID NO: 77 - RNA, 20 nt, synthetic construct
  misc_feature 1..20: note = Synthetic
  source 1..20: mol_type = other RNA
ccgggagcagatgacggcca

SEQ ID NO: 78 - RNA, 20 nt, synthetic construct
  misc_feature 1..20: note = Synthetic
  source 1..20: mol_type = other RNA
acgttacctcgtgcggcccg

SEQ ID NO: 79 - RNA, 20 nt, synthetic construct
  misc_feature 1..20: note = Synthetic
  source 1..20: mol_type = other RNA
attgtctttcctagcggaat

SEQ ID NO: 80 - RNA, 20 nt, synthetic construct
  misc_feature 1..20: note = Synthetic
  source 1..20: mol_type = other RNA
tcagtggctgggcactccga

SEQ ID NO: 81 - RNA, 20 nt, synthetic construct
  misc_feature 1..20: note = Synthetic
  source 1..20: mol_type = other RNA
tggcatactccgtctgctca

SEQ ID NO: 82 - RNA, 20 nt, synthetic construct
  misc_feature 1..20: note = Synthetic
  source 1..20: mol_type = other RNA
acctcatcccccgcccgcag

SEQ ID NO: 83 - RNA, 20 nt, synthetic construct
  misc_feature 1..20: note = Synthetic
  source 1..20: mol_type = other RNA
accgcccagacgactggcca

SEQ ID NO: 84 - RNA, 20 nt, synthetic construct
  misc_feature 1..20: note = Synthetic
  source 1..20: mol_type = other RNA
gggctcatgcggtaccagtt

SEQ ID NO: 85 - RNA, 20 nt, synthetic construct
  misc_feature 1..20: note = Synthetic
  source 1..20: mol_type = other RNA
acgactggccagggcgcctg

SEQ ID NO: 86 - RNA, 20 nt, synthetic construct
  misc_feature 1..20: note = Synthetic
  source 1..20: mol_type = other RNA
ccggggtcttctctcgccac

SEQ ID NO: 87 - RNA, 20 nt, synthetic construct
  misc_feature 1..20: note = Synthetic
  source 1..20: mol_type = other RNA
gcagcctggtgctgctagtc

SEQ ID NO: 88 - RNA, 20 nt, synthetic construct
  misc_feature 1..20: note = Synthetic
  source 1..20: mol_type = other RNA
agccggccagttccaaaccc

SEQ ID NO: 89 - protein, 7 aa, synthetic construct
  REGION 1..7: note = Synthetic
PKKKRKV

SEQ ID NO: 90 - protein, 7 aa, synthetic construct
  REGION 1..7: note = Synthetic
PKKKRRV

SEQ ID NO: 91 - protein, 16 aa, synthetic construct
  REGION 1..16: note = Synthetic
KRPAATKKAGQAKKKK

SEQ ID NO: 92 - protein, 7 aa, synthetic construct
  REGION 1..7: note = Synthetic
PKKKRKV

SEQ ID NO: 93-100 - skipped (8 entries)

SEQ ID NO: 101 - RNA, 100 nt, synthetic construct
  misc_feature 1..100: note = Synthetic
  source 1..100: mol_type = other RNA
cgtgtcacacaactgcccaagttttagagctagaaatagcaagttaaaataaggctagtccgttatcaacttgaaaaagtggcaccgagtcggtgctttt

SEQ ID NO: 102 - RNA, 100 nt, synthetic construct
  misc_feature 1..100: note = Synthetic
  source 1..100: mol_type = other RNA
cccttcggtcaccacgagcagttttagagctagaaatagcaagttaaaataaggctagtccgttatcaacttgaaaaagtggcaccgagtcggtgctttt

SEQ ID NO: 103 - RNA, 100 nt, synthetic construct
  misc_feature 1..100: note = Synthetic
  source 1..100: mol_type = other RNA
gcagttgtgtgacacggaaggttttagagctagaaatagcaagttaaaataaggctagtccgttatcaacttgaaaaagtggcaccgagtcggtgctttt

SEQ ID NO: 104 - RNA, 100 nt, synthetic construct
  misc_feature 1..100: note = Synthetic
  source 1..100: mol_type = other RNA
cgactggccagggcgcctgtgttttagagctagaaatagcaagttaaaataaggctagtccgttatcaacttgaaaaagtggcaccgagtcggtgctttt

SEQ ID NO: 105 - RNA, 100 nt, synthetic construct
  misc_feature 1..100: note = Synthetic
  source 1..100: mol_type = other RNA
cgtctgggcggtgctacaacgttttagagctagaaatagcaagttaaaataaggctagtccgttatcaacttgaaaaagtggcaccgagtcggtgctttt

SEQ ID NO: 106 - RNA, 100 nt, synthetic construct
  misc_feature 1..100: note = Synthetic
  source 1..100: mol_type = other RNA
gccctgctcgtggtgaccgagttttagagctagaaatagcaagttaaaataaggctagtccgttatcaacttgaaaaagtggcaccgagtcggtgctttt

SEQ ID NO: 107 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
ccaatatcaggagactaggagttttagagctagaaatagcaagttaaaataaggctagtccgttatcaacttgaaaaagtggcaccgagtcggtgctttt

SEQ ID NO: 108 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
gaccccctccaccccgcctcgttttagagctagaaatagcaagttaaaataaggctagtccgttatcaacttgaaaaagtggcaccgagtcggtgctttt

SEQ ID NO: 109 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
ggcctcggcgctgacgatctgttttagagctagaaatagcaagttaaaataaggctagtccgttatcaacttgaaaaagtggcaccgagtcggtgctttt

SEQ ID NO: 110 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
ctctcagctggtacacggcagttttagagctagaaatagcaagttaaaataaggctagtccgttatcaacttgaaaaagtggcaccgagtcggtgctttt

SEQ ID NO: 111 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
agttgggcagataacacttggttttagagctagaaatagcaagttaaaataaggctagtccgttatcaacttgaaaaagtggcaccgagtcggtgctttt

SEQ ID NO: 112 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
gatcacgtcggccgttggcggttttagagctagaaatagcaagttaaaataaggctagtccgttatcaacttgaaaaagtggcaccgagtcggtgctttt

SEQ ID NO: 113 - RNA, 100 nt, synthetic construct; identical to SEQ ID NO: 104
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA

SEQ ID NO: 114 - RNA, 100 nt, synthetic construct
  misc_feature 1..100: note = Synthetic
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
cacctacctaagaaccatccgttttagagctagaaatagcaagttaaaataaggctagtccgttatcaacttgaaaaagtggcaccgagtcggtgctttt

SEQ ID NO: 115 - RNA, 42 nt, synthetic construct
  misc_feature 1..42: note = Synthetic
  source 1..42: mol_type = other RNA
gaccccctccaccccgcctcgttttagagctatgctgttttg

SEQ ID NO: 116 - RNA, 25 nt, synthetic construct
  misc_feature 1..25: note = Synthetic
  source 1..25: mol_type = other RNA
tggctgggcacgcgtttaatataag

SEQ ID NO: 117 - RNA, 25 nt, synthetic construct
  misc_feature 1..25: note = Synthetic
  source 1..25: mol_type = other RNA
ctgggcacgcgtttaatataagtgg

SEQ ID NO: 118 - RNA, 25 nt, synthetic construct
  misc_feature 1..25: note = Synthetic
  source 1..25: mol_type = other RNA
tttaatataagtggaggcgtcgcgc

SEQ ID NO: 119 - RNA, 25 nt, synthetic construct
  misc_feature 1..25: note = Synthetic
  source 1..25: mol_type = other RNA
aatataagtggaggcgtcgcgctgg

SEQ ID NO: 120 - RNA, 25 nt, synthetic construct
  misc_feature 1..25: note = Synthetic
  source 1..25: mol_type = other RNA
atataagtggaggcgtcgcgctggc

SEQ ID NO: 121 - RNA, 25 nt, synthetic construct
  misc_feature 1..25: note = Synthetic
  source 1..25: mol_type = other RNA
gggcattcctgaagctgacagcatt

SEQ ID NO: 122 - RNA, 25 nt, synthetic construct
  misc_feature 1..25: note = Synthetic
  source 1..25: mol_type = other RNA
ggcattcctgaagctgacagcattc

SEQ ID NO: 123 - RNA, 25 nt, synthetic construct
  misc_feature 1..25: note = Synthetic
  source 1..25: mol_type = other RNA
attcgggccgagatgtctcgctccg

SEQ ID NO: 124 - RNA, 25 nt, synthetic construct
  misc_feature 1..25: note = Synthetic
  source 1..25: mol_type = other RNA
ctgtgctcgcgctactctctctttc

SEQ ID NO: 125 - RNA, 25 nt, synthetic construct
  misc_feature 1..25: note = Synthetic
  source 1..25: mol_type = other RNA
ctcgcgctactctctctttctggcc

SEQ ID NO: 126 - RNA, 25 nt, synthetic construct
  misc_feature 1..25: note = Synthetic
  source 1..25: mol_type = other RNA
gcgctactctctctttctggcctgg

SEQ ID NO: 127 - RNA, 25 nt, synthetic construct
  misc_feature 1..25: note = Synthetic
  source 1..25: mol_type = other RNA
atattaaacgcgtgcccagccaatc

SEQ ID NO: 128 - RNA, 25 nt, synthetic construct
  misc_feature 1..25: note = Synthetic
  source 1..25: mol_type = other RNA
tctcggcccgaatgctgtcagcttc

SEQ ID NO: 129 - RNA, 25 nt, synthetic construct
  misc_feature 1..25: note = Synthetic
  source 1..25: mol_type = other RNA
gctaaggccacggagcgagacatct

SEQ ID NO: 130 - RNA, 25 nt, synthetic construct
  misc_feature 1..25: note = Synthetic
  source 1..25: mol_type = other RNA
agtagcgcgagcacagctaaggcca

SEQ ID NO: 131 - RNA, 25 nt, synthetic construct
  misc_feature 1..25: note = Synthetic
  source 1..25: mol_type = other RNA
agagagagtagcgcgagcacagcta

SEQ ID NO: 132 - RNA, 25 nt, synthetic construct
  misc_feature 1..25: note = Synthetic
  source 1..25: mol_type = other RNA
gagagactcacgctggatagcctcc

SEQ ID NO: 133 - RNA, 25 nt, synthetic construct
  misc_feature 1..25: note = Synthetic
  source 1..25: mol_type = other RNA
gcgggagggtaggagagactcacgc

SEQ ID NO: 134 - RNA, 25 nt, synthetic construct
  misc_feature 1..25: note = Synthetic
  source 1..25: mol_type = other RNA
tattcctcaggtactccaaagattc

SEQ ID NO: 135 - RNA, 25 nt, synthetic construct
  misc_feature 1..25: note = Synthetic
  source 1..25: mol_type = other RNA
tttactcacgtcatccagcagagaa

SEQ ID NO: 136 - RNA, 25 nt, synthetic construct
  misc_feature 1..25: note = Synthetic
  source 1..25: mol_type = other RNA
caaatttcctgaattgctatgtgtc

SEQ ID NO: 137 - RNA, 25 nt, synthetic construct
  misc_feature 1..25: note = Synthetic
  source 1..25: mol_type = other RNA
aaatttcctgaattgctatgtgtct

SEQ ID NO: 138 - RNA, 25 nt, synthetic construct
  misc_feature 1..25: note = Synthetic
  source 1..25: mol_type = other RNA
acattgaagttgacttactgaagaa

SEQ ID NO: 139 - RNA, 25 nt, synthetic construct
  misc_feature 1..25: note = Synthetic
  source 1..25: mol_type = other RNA
aagaatggagagagaattgaaaaag

SEQ ID NO: 140 - RNA, 25 nt, synthetic construct
  misc_feature 1..25: note = Synthetic
  source 1..25: mol_type = other RNA
gagcattcagacttgtctttcagca

SEQ ID NO: 141 - RNA, 25 nt, synthetic construct
  misc_feature 1..25: note = Synthetic
  source 1..25: mol_type = other RNA
ttcagacttgtctttcagcaaggac

SEQ ID NO: 142 - RNA, 25 nt, synthetic construct
  misc_feature 1..25: note = Synthetic
  source 1..25: mol_type = other RNA
tttgtcacagcccaagatagttaag

SEQ ID NO: 143 - RNA, 25 nt, synthetic construct
  misc_feature 1..25: note = Synthetic
  source 1..25: mol_type = other RNA
ttgtcacagcccaagatagttaagt

SEQ ID NO: 144 - RNA, 25 nt, synthetic construct
  misc_feature 1..25: note = Synthetic
  source 1..25: mol_type = other RNA
tgtcacagcccaagatagttaagtg

SEQ ID NO: 145 - RNA, 25 nt, synthetic construct
  misc_feature 1..25: note = Synthetic
  source 1..25: mol_type = other RNA
atctttggagtacctgaggaatatc

SEQ ID NO: 146 - RNA, 25 nt, synthetic construct
  misc_feature 1..25: note = Synthetic
  source 1..25: mol_type = other RNA
aatctttggagtacctgaggaatat

SEQ ID NO: 147 - RNA, 25 nt, synthetic construct
  misc_feature 1..25: note = Synthetic
  source 1..25: mol_type = other RNA
taaacctgaatctttggagtacctg

SEQ ID NO: 148 - RNA, 25 nt, synthetic construct
  misc_feature 1..25: note = Synthetic
  source 1..25: mol_type = other RNA
gatgacgtgagtaaacctgaatctt

SEQ ID NO: 149 - RNA, 25 nt, synthetic construct
  misc_feature 1..25: note = Synthetic
  source 1..25: mol_type = other RNA
ggaaatttgactttccattctctgc

SEQ ID NO: 150 - RNA, 25 nt, synthetic construct
  misc_feature 1..25: note = Synthetic
  source 1..25: mol_type = other RNA
atgaaacccagacacatagcaattc

SEQ ID NO: 151 - RNA, 25 nt, synthetic construct
  misc_feature 1..25: note = Synthetic
  source 1..25: mol_type = other RNA
tcagtaagtcaacttcaatgtcgga

SEQ ID NO: 152 - RNA, 25 nt, synthetic construct
  misc_feature 1..25: note = Synthetic
  source 1..25: mol_type = other RNA
ttcttcagtaagtcaacttcaatgt

SEQ ID NO: 153 - RNA, 25 nt, synthetic construct
  misc_feature 1..25: note = Synthetic
  source 1..25: mol_type = other RNA
caggcatactcatctttttcagtgg

SEQ ID NO: 154 - RNA, 25 nt, synthetic construct
  misc_feature 1..25: note = Synthetic
  source 1..25: mol_type = other RNA
gcaggcatactcatctttttcagtg

SEQ ID NO: 155 - RNA, 25 nt, synthetic construct
  misc_feature 1..25: note = Synthetic
  source 1..25: mol_type = other RNA
ggcaggcatactcatctttttcagt

SEQ ID NO: 156 - RNA, 25 nt, synthetic construct
  misc_feature 1..25: note = Synthetic
  source 1..25: mol_type = other RNA
cggcaggcatactcatctttttcag

SEQ ID NO: 157 - RNA, 25 nt, synthetic construct
  misc_feature 1..25: note = Synthetic
  source 1..25: mol_type = other RNA
gacaaagtcacatggttcacacggc

SEQ ID NO: 158 - RNA, 25 nt, synthetic construct
  misc_feature 1..25: note = Synthetic
  source 1..25: mol_type = other RNA
ctgtgacaaagtcacatggttcaca

SEQ ID NO: 159 - RNA, 25 nt, synthetic construct
  misc_feature 1..25: note = Synthetic
  source 1..25: mol_type = other RNA
tatcttgggctgtgacaaagtcaca

SEQ ID NO: 160 - RNA, 25 nt, synthetic construct
  misc_feature 1..25: note = Synthetic
  source 1..25: mol_type = other RNA
aagacttaccccacttaactatctt

SEQ ID NO: 161 - RNA, 25 nt, synthetic construct
  misc_feature 1..25: note = Synthetic
  source 1..25: mol_type = other RNA
taagacttaccccacttaactatct

SEQ ID NO: 162 - RNA, 25 nt, synthetic construct
  misc_feature 1..25: note = Synthetic
  source 1..25: mol_type = other RNA
agatcgagacatgtaagcagcatca

SEQ ID NO: 163 - RNA, 25 nt, synthetic construct
  misc_feature 1..25: note = Synthetic
  source 1..25: mol_type = other RNA
tcgagacatgtaagcagcatcatgg

SEQ ID NO: 164 - RNA, 25 nt, synthetic construct
  misc_feature 1..25: note = Synthetic
  source 1..25: mol_type = other RNA
atgtctcgatctatgaaaaagacag

SEQ ID NO: 165 - RNA, 25 nt, synthetic construct
  misc_feature 1..25: note = Synthetic
  source 1..25: mol_type = other RNA
ttttcaggtttgaagatgccgcatt

SEQ ID NO: 166 - RNA, 25 nt, synthetic construct
  misc_feature 1..25: note = Synthetic
  source 1..25: mol_type = other RNA
aggtttgaagatgccgcatttggat

SEQ ID NO: 167 - RNA, 25 nt, synthetic construct
  misc_feature 1..25: note = Synthetic
  source 1..25: mol_type = other RNA
cacttacactttatgcacaaaatgt

SEQ ID NO: 168 - RNA, 25 nt, synthetic construct
  misc_feature 1..25: note = Synthetic
  source 1..25: mol_type = other RNA
acttacactttatgcacaaaatgta

SEQ ID NO: 169 - RNA, 25 nt, synthetic construct
  misc_feature 1..25: note = Synthetic
  source 1..25: mol_type = other RNA
atgtagggttataataatgttaaca

SEQ ID NO: 170 - RNA, 25 nt, synthetic construct
  misc_feature 1..25: note = Synthetic
  source 1..25: mol_type = other RNA
gtctccatgtttgatgtatctgagc

SEQ ID NO: 171 - RNA, 25 nt, synthetic construct
  misc_feature 1..25: note = Synthetic
  source 1..25: mol_type = other RNA
gatgtatctgagcaggttgctccac

SEQ ID NO: 172 - RNA, 25 nt, synthetic construct
  misc_feature 1..25: note = Synthetic
  source 1..25: mol_type = other RNA
agcaggttgctccacaggtagctct

SEQ ID NO: 173 - RNA, 25 nt, synthetic construct
  misc_feature 1..25: note = Synthetic
  source 1..25: mol_type = other RNA
aggttgctccacaggtagctctagg

SEQ ID NO: 174 - RNA, 25 nt, synthetic construct
  misc_feature 1..25: note = Synthetic
  source 1..25: mol_type = other RNA
ggttgctccacaggtagctctagga

SEQ ID NO: 175 - RNA, 25 nt, synthetic construct
  misc_feature 1..25: note = Synthetic
  source 1..25: mol_type = other RNA
gctccacaggtagctctaggagggc

SEQ ID NO: 176 - RNA, 25 nt, synthetic construct
  misc_feature 1..25: note = Synthetic
  source 1..25: mol_type = other RNA
agctctaggagggctggcaacttag

SEQ ID NO: 177 - RNA, 25 nt, synthetic construct
  misc_feature 1..25: note = Synthetic
  source 1..25: mol_type = other RNA
tctaggagggctggcaacttagagg

SEQ ID NO: 178 - RNA, 25 nt, synthetic construct
  misc_feature 1..25: note = Synthetic
  source 1..25: mol_type = other RNA
ctaggagggctggcaacttagaggt

SEQ ID NO: 179 - RNA, 25 nt, synthetic construct
  misc_feature 1..25: note = Synthetic
  source 1..25: mol_type = other RNA
taggagggctggcaacttagaggtg

SEQ ID NO: 180 - RNA, 25 nt, synthetic construct
  misc_feature 1..25: note = Synthetic
  source 1..25: mol_type = other RNA
attctcttatccaacatcaacatct

SEQ ID NO: 181 - RNA, 25 nt, synthetic construct
  misc_feature 1..25: note = Synthetic
  source 1..25: mol_type = other RNA
caatttacatactctgcttagaatt

SEQ ID NO: 182 - RNA, 25 nt, synthetic construct
  misc_feature 1..25: note = Synthetic
  source 1..25: mol_type = other RNA
aatttacatactctgcttagaattt

SEQ ID NO: 183 - RNA, 25 nt, synthetic construct
  misc_feature 1..25: note = Synthetic
  source 1..25: mol_type = other RNA
atttacatactctgcttagaatttg

SEQ ID NO: 184 - RNA, 25 nt, synthetic construct
  misc_feature 1..25: note = Synthetic
  source 1..25: mol_type = other RNA
tttacatactctgcttagaatttgg

SEQ ID NO: 185 - RNA, 25 nt, synthetic construct
  misc_feature 1..25: note = Synthetic
  source 1..25: mol_type = other RNA
gggaaaatttagaaatataattgac

SEQ ID NO: 186 - RNA, 25 nt, synthetic construct
  misc_feature 1..25: note = Synthetic
  source 1..25: mol_type = other RNA
ttagaaatataattgacaggattat

SEQ ID NO: 187 - RNA, 25 nt, synthetic construct
  misc_feature 1..25: note = Synthetic
  source 1..25: mol_type = other RNA
tacttcttatacatttgataaagta

SEQ ID NO: 188 - RNA, 25 nt, synthetic construct
  misc_feature 1..25: note = Synthetic
  source 1..25: mol_type = other RNA
cttatacatttgataaagtaaggca

SEQ ID NO: 189 - RNA, 25 nt, synthetic construct
  misc_feature 1..25: note = Synthetic
  source 1..25: mol_type = other RNA
catttgataaagtaaggcatggttg

SEQ ID NO: 190 - RNA, 25 nt, synthetic construct
  misc_feature 1..25: note = Synthetic
  source 1..25: mol_type = other RNA
aagtaaggcatggttgtggttaatc

SEQ ID NO: 191 - RNA, 25 nt, synthetic construct
  misc_feature 1..25: note = Synthetic
  source 1..25: mol_type = other RNA
cttcaaacctgaaaagaaaagaaaa

SEQ ID NO: 192 - RNA, 25 nt, synthetic construct
  misc_feature 1..25: note = Synthetic
  source 1..25: mol_type = other RNA
atttggaattcatccaatccaaatg

SEQ ID NO: 193 - RNA, 25 nt, synthetic construct
  misc_feature 1..25: note = Synthetic
  source 1..25: mol_type = other RNA
tattaaaaagcaagcaagcagaatt

SEQ ID NO: 194 - RNA, 25 nt, synthetic construct
  misc_feature 1..25: note = Synthetic
  source 1..25: mol_type = other RNA
gcaacctgctcagatacatcaaaca

SEQ ID NO: 195 - RNA, 25 nt, synthetic construct
  misc_feature 1..25: note = Synthetic
  source 1..25: mol_type = other RNA
ttgccagccctcctagagctacctg

SEQ ID NO: 196 - RNA, 25 nt, synthetic construct
  misc_feature 1..25: note = Synthetic
  source 1..25: mol_type = other RNA
tcaaatctgaccaagatgttgatgt

SEQ ID NO: 197 - RNA, 25 nt, synthetic construct
  misc_feature 1..25: note = Synthetic
  source 1..25: mol_type = other RNA
caaattctaagcagagtatgtaaat

SEQ ID NO: 198 - RNA, 25 nt, synthetic construct
  misc_feature 1..25: note = Synthetic
  source 1..25: mol_type = other RNA
caagttttatgatttatttaacttg

SEQ ID NO: 199 - skipped

SEQ ID NO: 200 - RNA, 22 nt, synthetic construct
  misc_feature 1..22: note = Synthetic
  source 1..22: mol_type = other RNA
gttttagagctatgctgttttg

SEQ ID NO: 201 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 202 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 203 - RNA, 42 nt, synthetic construct
  misc_feature 1..42: note = Synthetic
  misc_feature 1..20: note = n is a, c, g, or u
  source 1..42: mol_type = other RNA
nnnnnnnnnnnnnnnnnnnngttttagagctatgctgttttg

SEQ ID NO: 204 - RNA, 74 nt, synthetic construct
  misc_feature 1..74: note = Synthetic
  source 1..74: mol_type = other RNA
aacagcatagcaagttaaaataaggctagtccgttatcaacttgaaaaagtggcaccgagtcggtgcttttttt

SEQ ID NO: 205-299 - skipped (95 entries)

SEQ ID NO: 300 - RNA, 100 nt, synthetic construct
  misc_feature 1..100: note = Synthetic
  misc_feature 1..20: note = n is a, c, g, or u
  modified_base 1..3: mod_base = OTHER; note = 2'-O-methyl and phosphorothioate linkage
  modified_base 29..40: mod_base = OTHER; note = 2'-O-methyl
  modified_base 69..100: mod_base = OTHER; note = 2'-O-methyl
  modified_base 97..100: mod_base = OTHER; note = phosphorothioate linkage
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 301-400 - skipped (100 entries)

SEQ ID NO: 401 - RNA, 90 nt, synthetic construct
  misc_feature 1..90: note = Synthetic
  misc_feature 1..20: note = n is a, c, g, or u
  source 1..90: mol_type = other RNA
nnnnnnnnnnnnnnnnnnnngttttagagctagaaatagcaagttaaaataaggctagtccgttatcacgaaagggcaccgagtcggtgc

SEQ ID NO: 402 - RNA, 90 nt, synthetic construct; identical to SEQ ID NO: 401
  misc_feature 1..90: note = Synthetic
  misc_feature 1..20: note = n is a, c, g, or u
  modified_base 1..3: mod_base = OTHER; note = 2'-O-methyl and phosphorothioate linkage
  modified_base 29..40: mod_base = OTHER; note = 2'-O-methyl
  modified_base 88..90: mod_base = OTHER; note = 2'-O-methyl
  modified_base 87..90: mod_base = OTHER; note = phosphorothioate linkage
  source 1..90: mol_type = other RNA

SEQ ID NO: 403 - RNA, 88 nt, synthetic construct
  misc_feature 1..88: note = Synthetic
  misc_feature 1..20: note = n is a, c, g, or u
  source 1..88: mol_type = other RNA
nnnnnnnnnnnnnnnnnnnngttttagagctagaaatagcaagttaaaataaggctagtccgttatcaacttggcaccgagtcggtgc

SEQ ID NO: 404 - RNA, 88 nt, synthetic construct; identical to SEQ ID NO: 403
  misc_feature 1..88: note = Synthetic
  misc_feature 1..20: note = n is a, c, g, or u
  modified_base 1..3: mod_base = OTHER; note = 2'-O-methyl and phosphorothioate linkage
  modified_base 29..40: mod_base = OTHER; note = 2'-O-methyl
  modified_base 86..88: mod_base = OTHER; note = 2'-O-methyl
  modified_base 85..88: mod_base = OTHER; note = phosphorothioate linkage
  source 1..88: mol_type = other RNA

SEQ ID NO: 405 - RNA, 88 nt, synthetic construct; identical to SEQ ID NO: 403
  misc_feature 1..88: note = Synthetic
  misc_feature 1..20: note = n is a, c, g, or u
  source 1..88: mol_type = other RNA

SEQ ID NO: 406 - RNA, 88 nt, synthetic construct; identical to SEQ ID NO: 403
  misc_feature 1..88: note = Synthetic
  misc_feature 1..20: note = n is a, c, g, or u
  modified_base 1..3: mod_base = OTHER; note = 2'-O-methyl and phosphorothioate linkage
  modified_base 29..40: mod_base = OTHER; note = 2'-O-methyl
  modified_base 86..88: mod_base = OTHER; note = 2'-O-methyl
  modified_base 85..88: mod_base = OTHER; note = phosphorothioate linkage
  source 1..88: mol_type = other RNA

SEQ ID NO: 407 - RNA, 90 nt, synthetic construct; identical to SEQ ID NO: 401
  misc_feature 1..90: note = Synthetic
  misc_feature 1..20: note = n is a, c, g, or u
  source 1..90: mol_type = other RNA

SEQ ID NO: 408 - RNA, 90 nt, synthetic construct; identical to SEQ ID NO: 401
  misc_feature 1..90: note = Synthetic
  misc_feature 1..20: note = n is a, c, g, or u
  modified_base 1..3: mod_base = OTHER; note = 2'-O-methyl and phosphorothioate linkage
  modified_base 21: mod_base = OTHER; note = 2'-O-methyl
  modified_base 24: mod_base = OTHER; note = 2'-fluoro
  modified_base 28..42: mod_base = OTHER; note = 2'-O-methyl
  modified_base 44: mod_base = OTHER; note = 2'-fluoro
  modified_base 46: mod_base = OTHER; note = 2'-O-methyl, 2'-fluoro
  modified_base 48: mod_base = OTHER; note = 2'-O-methyl, 2'-fluoro
  modified_base 50: mod_base = OTHER; note = 2'-O-methyl
  modified_base 52..57: mod_base = OTHER; note = 2'-O-methyl
  modified_base 60..61: mod_base = OTHER; note = 2'-O-methyl
  modified_base 63: mod_base = OTHER; note = 2'-fluoro
  modified_base 66..67: mod_base = OTHER; note = 2'-O-methyl
  modified_base 69..90: mod_base = OTHER; note = 2'-O-methyl
  modified_base 87..90: mod_base = OTHER; note = phosphorothioate linkage
  source 1..90: mol_type = other RNA

SEQ ID NO: 409 - RNA, 90 nt, synthetic construct; identical to SEQ ID NO: 401
  misc_feature 1..90: note = Synthetic
  misc_feature 1..20: note = n is a, c, g, or u
  source 1..90: mol_type = other RNA

SEQ ID NO: 410 - RNA, 90 nt, synthetic construct; identical to SEQ ID NO: 401
  misc_feature 1..90: note = Synthetic
  misc_feature 1..20: note = n is a, c, g, or u
  modified_base 1..3: mod_base = OTHER; note = 2'-O-methyl and phosphorothioate linkage
  modified_base 29..40: mod_base = OTHER; note = 2'-O-methyl
  modified_base 88..90: mod_base = OTHER; note = 2'-O-methyl
  modified_base 87..90: mod_base = OTHER; note = phosphorothioate linkage
  source 1..90: mol_type = other RNA

SEQ ID NO: 411-799 - skipped (389 entries)

SEQ ID NO: 800 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 801 - DNA, 4140 bp, synthetic construct
  misc_feature 1..4140: note = Synthetic
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 802 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 803 - RNA, 4197 nt, synthetic construct
  misc_feature 1..4197: note = Synthetic
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 804 - RNA, 4140 nt, synthetic construct
  misc_feature 1..4140: note = Synthetic
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 805 - RNA, 4140 nt, synthetic construct
  misc_feature 1..4140: note = Synthetic
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 806-1000 - skipped (195 entries)

SEQ ID NO: 1001 - DNA, 4607 bp, synthetic construct
  misc_feature 1..4607: note = Synthetic
  source 1..4607: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

(19) United States
(12) Patent Application Publication
(10) Pub. No.: US 2024/0228577 A1
(43) Pub. Date: Jul. 11, 2024

(54) Programmed Cell Death Protein 1 (PD1) Compositions and Methods for Cell-Based Therapy

(71) Applicant: Intellia Therapeutics, Inc., Cambridge, MA (US)

(72) Inventor: Danielle Ryan Cook, Boston, MA (US)

(73) Assignee: Intellia Therapeutics, Inc., Cambridge, MA (US)

(21) Appl. No.: 18/585,231

(22) Filed: Feb. 23, 2024

Related U.S. Application Data
(63) Continuation of application No. PCT/US2022/075317, filed on Aug. 23, 2022.
(60) Provisional application No. 63/236,398, filed on Aug. 24, 2021.

(51) Int. Cl. C07K 14/705; A61K 39/00; C07K 14/725; C07K 14/74; C12N 5/0783; C12N 9/22; C12N 15/113; C12N 15/88

(52) U.S. Cl. CPC C07K 14/70521; A61K 39/4611; A61K 39/4632; A61K 39/4637; C07K 14/70503; C07K 14/7051; C07K 14/70539; C12N 5/0636; C12N 9/22; C12N 15/1138; C12N 15/88; C12N 2310/20; C12N 2310/315; C12N 2310/321; C12N 2310/322; C12N 2310/531; C12N 2510/00

(57) ABSTRACT

Compositions and methods for editing, e.g., altering a DNA sequence, within a PD1 gene are provided. Compositions and methods for cell-based therapy are provided.

This application is a continuation of International Application No. PCT/US2022/075317, filed Aug. 23, 2022, which claims the benefit under 35 U.S.C. 119(e) of U.S. Provisional Application No. 63/236,398, filed Aug. 24, 2021, the content of each of which is incorporated herein by reference in its entirety.

This application is filed with a Sequence Listing which has been submitted electronically in XML format. The Sequence Listing is provided as a file entitled “01155-0044-00PCT_ST26.xml” created on Aug. 17, 2022, which is 495,292 bytes in size. The information in the electronic format of the Sequence Listing is incorporated herein by reference in its entirety.

T cell exhaustion is a broad term that has been used to describe the response of T cells to chronic antigen stimulation. This was first observed in the setting of chronic viral infection but has also been studied in the immune response to tumors. The features and characteristics of the T-cell exhaustion mechanism may have crucial implications for the success of checkpoint blockade and adoptive T cell transfer therapies.

T cell exhaustion is a progressive loss of effector function due to prolonged antigen stimulation, characteristic of chronic infections and cancer. In addition to continuous antigen stimulation, antigen presenting cells and cytokines present in the microenvironment can also contribute to this exhausted phenotype. Thus T cell exhaustion is a state of T cell dysfunction in which T cells present poor effector function and sustained expression of inhibitory receptors. This prevents optimal control of infections or tumours. Additionally, exhausted T cells have a transcriptional state distinct from that of functional effector or memory T cells. Therapeutic treatments have the potential to rescue exhausted T cells (Goldberg, M. V. & Drake, C. G., 2011, Wherry, E. J. & Kurachi M., 2015).

Exhausted T cells typically express co-inhibitory receptors such as programmed cell death 1 (PD1). The gene product acts as a component of an immune checkpoint system. T cell exhaustion may be reversed by blocking these receptors.

### SUMMARY

Provided herein are compounds and compositions for use, for example, in methods of preparation of cells with genetic modifications (e.g., insertions, deletions, substitutions) in a PD1 sequence, e.g., a genomic locus, generated, for example, using the CRISPR/Cas system; and the cells with genetic modifications in the PD1 sequence and their use in various methods, e.g., to promote an immune response e.g., in immunooncology and infectious disease. The cells with PD1 genetic modifications that may reduce PD1 expression, may include genetic modifications in additional genomic sequences including. T-cell receptor (TCR) loci, e.g., TRAC or TRBC loci, to reduce TCR expression: genomic loci that reduce expression of MHC class I molecules, e.g., B2M and HLA-A loci: genomic loci that reduce expression of MHC class II molecules, e.g., CIITA loci; and checkpoint inhibitor loci, e.g., CD244 (2B4) loci, TIM3 loci, and LAG3 loci. The present disclosure relates to populations of cells including cells with genetic modification of the PD1 sequence, and optionally other genomic loci as provided herein. The cells may be used in adoptive T cell transfer therapies. The present disclosure relates to compositions and uses of the cells with genetic modification of the PD1 sequence for use in therapy, e.g., cancer therapy and immunotherapy. The present disclosure relates to and provides gRNA molecules, CRISPR systems, cells, and methods useful for genome editing of cells.

Also provided herein is an engineered cell comprising a genetic modification in a human PD1 sequence, within the genomic coordinates of chr12: 241849881-241858908. Further embodiments are provided throughout and described in the claims and Figures. A PD1 guide RNA that specifically hybridizes to a PD1 sequence comprising a nucleotide sequence selected from SEQ ID NO: 1-88.

Also disclosed is the use of a composition or formulation of a cell of any of the foregoing embodiments for the preparation of a medicament for treating a subject. The subject may be human or animal (e.g., human or non-human animal, e.g., cynomolgus monkey).

Also disclosed are any of the foregoing compositions or formulations for use in producing a genetic modification (e.g., an insertion, a substitution, or a deletion) a PD1 gene sequence.

Also disclosed is a PD1 guide RNA that specifically hybridizes to a PD1 sequence, comprising a guide sequences disclosed herein. Also disclosed is a PD1 guide RNA comprising a guide sequence that directs an RNA-guided DNA binding agent to a chromosomal location within the genomic coordinates disclosed herein.

### BRIEF DESCRIPTION OF THE DRAWINGS

FIG. 1 shows the extent of PD1 protein expression in T-cells as measured by flow cytometry from each of 4 donors (“100”, “112”, “315”, and “797”). The y-axis shows the percentage of PD1 positive cells with the error bars showing the standard deviation (SD) of this measurement.

FIGS. 2A-2B show the extent of editing in restimulated T-cells as measured by NGS sequencing (FIG. 2A) and the percent of PD1+ cells as measured by flow cytometry (FIG. 2B) with the error bars showing the SD of this measurement.

FIG. 3 shows a dose response curve of editing with PD1 guide RNAs as well as control guide RNAs at the respective targeting loci in T cells.

### DETAILED DESCRIPTION

Reference will now be made in detail to certain embodiments of the disclosure, examples of which are illustrated in the accompanying drawings. While the present teachings are described in conjunction with various embodiments, it is not intended to limit the present teachings to those embodiments. On the contrary, the present teaching encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended claims, the singular form “a”, “an”, and “the” include plural references unless the context clearly dictates otherwise. Thus, for example, reference to “a conjugate” includes a plurality of conjugates and reference to “a cell” includes a plurality of cells (e.g., a population of cells) and the like.

Numeric ranges are inclusive of the numbers defining the range. Measured and measurable values are understood to be approximate, taking into account significant digits and the error associated with the measurement. In some embodiments a population of cells refers to a population of at least 10³, 10⁴, 10⁵ or 10⁶ cells, preferably 10⁷, 2×10⁷, 5×10⁷, or 10⁸ cells.

The use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings. Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense in the specification, i.e., equivalent to “and/or,” unless the context clearly indicates otherwise.

The term “about”, when used before a list, modifies each member of the list. The term “about” is understood to encompass tolerated variation or error within the art, e.g., 2 standard deviations from the mean, or the sensitivity of the method used to take a measurement. When “about” is present before the first value of a series, it can be understood to modify each value in the series.

Ranges are understood to include the numbers at the end of the range and all logical values therebetween. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least”, “up to”, or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

In the event of a conflict between a sequence in the application and an indicated accession number or position in an accession number, the sequence in the application predominates.

In the event of a conflict between a chemical name and a structure, the structure predominates.

As used herein, “detecting an analyte” and the like is understood as performing an assay in which the analyte can be detected, if present, wherein the analyte is present in an amount above the level of detection of the assay.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

The section headings used herein are for organizational purposes only and are not to be construed as limiting the desired subject matter in any way. In the event that any material incorporated by reference contradicts any term defined in this specification or any other express content of this specification, this specification controls.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. An RNA may comprise one or more deoxyribose nucleotides, e.g. as modifications, and similarly a DNA may comprise one or more ribonucleotides. Nitrogenous bases can be conventional bases (A, G. C. T. U), analogs thereof (e.g., modified uridines such as 5-methoxyuridine, pseudouridine, or N1-methylpseudouridine, or others): inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines: U.S. Pat. No. 5,378,825 and PCT No. WO 93/13121). For general discussion see The Biochemistry of the Nucleic Acids 5-36. Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleosides and one or more nucleoside analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel. 2004. Biochemistry 43(42): 13233-41). RNA and DNA have different sugar moieties and can differ by the presence of uracil or analogs thereof in RNA and thymine or analogs thereof in DNA.

“Guide RNA”. “gRNA”, and simply “guide” are used herein interchangeably to refer to, for example, either a single guide RNA or the combination of a crRNA and a trRNA (also known as tracrRNA). The crRNA and trRNA may be associated as a single RNA molecule (as a single guide RNA, sgRNA) or, for example, in two separate RNA strands (dual guide RNA, dgRNA). “Guide RNA” or “gRNA” refers to each type. The trRNA may be a naturally-occurring sequence, or a trRNA sequence with modifications or variations. As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence.” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs: 1-88. In some embodiments, the target sequence is in a gene or on a chromosome, for example, and is complementary to the guide sequence. In some embodiments, the degree of complementarity or identity between a guide sequence and its corresponding target sequence is at least 75%, 80%, 85%, 90%, or 95%, or is 100%. For example, in some embodiments, the guide sequence comprises a sequence with at least 75%, 80%. 85%. 90%, or 95%, or 100% identity to at least 17, 18, 19, or 20 contiguous nucleotides of a sequence selected from SEQ ID NOs: 1-88. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1, 2, 3, or 4 mismatches, where the total length of the target sequence is 17, 18, 19, 20 nucleotides, or more. In some embodiments, the guide sequence and the target region may contain 1-4 mismatches where the guide sequence comprises at least 17, 18, 19, 20 nucleotides, or more. In some embodiments, the guide sequence and the target region may contain 1, 2, 3, or 4 mismatches where the guide sequence comprises 20 nucleotides. That is, the guide sequence and the target region may form a duplex region having 17, 18, 19, 20 base pairs, or more. In certain embodiments, the duplex region may include 1, 2, 3, or 4 mismatches such that guide strand and target sequence are not fully complementary. For example, a guide strand and target sequence may be complementary over a 20 nucleotide region, including 2 mismatches, such that the guide sequence and target sequence are 90% complementary providing a duplex region of 18 base pairs out of 20.

Target sequences for RNA-guided DNA binding agents include both the positive and negative strands of genomic DNA (i.e., the sequence given and the reverse complement of the sequence), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, as used herein, encompasses Cas cleavases. Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g. via fusion with a FokI domain. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants). HypaCas) (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015): Shmakov et al., Molecular Cell, 60:385-397 (2015).

Exemplary nucleotide and polypeptide sequences of Cas9 molecules are provided below: Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, amino acid sequences, or nucleic acid sequences encoding the amino acid sequences provided herein are also contemplated.

Exemplary open reading frame for Cas9 are provided in Table 12.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence: in cases where the agent is a cleavase or nickase, binding can be followed by cleaving or nicking.

As used herein, a “target sequence” refers to a sequence of nucleic acid in a target gene that has complementarity to the guide sequence of the gRNA, i.e., that is sufficiently complementary to the guide sequence to permit specific binding of the guide sequence. The interaction of the target sequence and the guide sequence directs an RNA-guided DNA binding agent to bind, and potentially nick or cleave (depending on the activity of the agent), within the target sequence.

As used herein, a first sequence is considered to be “identical” or have “100% identity” with a second sequence if an alignment of the first sequence to the second sequence shows that all of the positions of the second sequence in its entirety are matched by the first sequence. For example, the sequence AAG has 100% identity to the sequence AAGA because an alignment would give 100% identity in that there are matches, without gaps, to all three positions of the first sequence. Less than 100% identity can be calculated using routine methods. For example ACG would have 67% identity with AAGA as two of the three positions of the first sequence are matches to the second sequence (⅔=67%). The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine: another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

Similarly, as used herein, a first sequence is considered to be “fully complementary” or 100% complementary” to a second sequence when all of the nucletodies of a first sequence are complementary to a second sequence, without gaps. For example, the sequence UCU would be considered to be fully complementary to the sequence AAGA as each of the nucleobases from the first sequence basepair with the nucleotides of the second sequence, without gaps. The sequence UGU would be considered to be 67% complementary to the sequence AAGA as two of the three nucleobases of the first sequence basepair with nucleobases of the second sequence. One skilled in the art will understand that algorithms are available with various parameter settings to determine percent complementarity for any pair of sequences using, e.g., the NCBI BLAST interface (blast.ncbi.nlm.nih.gov/Blast.cgi) or the Needleman-Wunsch algorithm.

“mRNA” is used herein to refer to a polynucleotide that comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Exemplary guide sequences useful in the guide RNA compositions and methods described herein are shown in Table 1 and throughout the application. For example, where Table 1 shows a guide sequence, this guide sequence may be used in a guide RNA to direct a RNA-guided DNA binding agent, e.g., a nuclease, such as a Cas nuclease, such as Cas9, to a target sequence. Target sequences are provided in Table 1 as genomic coordinates, and include both the positive and negative strands of genomic DNA (i.e., the sequence given and the sequence's reverse complement. In some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “indels” refer to insertion/deletion mutations consisting of a number of nucleotides that are either inserted or deleted at the site of double-stranded breaks (DSBs) in a target nucleic acid.

As used herein, “inhibit expression” and the like refer to a decrease in expression of a particular gene product (e.g., protein, mRNA, or both). Expression of a protein (i.e., gene product) can be measured by detecting total cellular amount of the protein from a tissue or cell population of interest by detecting expression of a protein as individual members of a population of cells, e.g., by cell sorting to define percent of cells expressing a protein, or expression of a protein in cells in aggregate, e.g., by ELISA or western blot. Inhibition of expression can result from genetic modification of a gene sequence, e.g., a genomic sequence, such that the full-length gene product, or any gene product, is no longer expressed. e.g. knockdown of the gene. Certain genetic modifications can result in the introduction of frameshift or nonsense mutations that prevent translation of the full-length gene product. Genetic modifications at a splice site, e.g., at a position sufficiently close to a splice acceptor site or a splice donor site to disrupt splicing, can prevent translation of the full-length protein. Inhibition of expression can result from a genetic modification in a regulatory sequence within the genomic sequence required for the expression of the gene product, e.g., a promoter sequence, a 3′ UTR sequence, e.g., a capping sequence, a 5′ UTR sequence, e.g., a poly A sequence. Inhibition of expression may also result from disrupting expression or activity of regulatory factors required for translation of the gene product, e.g., production of no gene product. For example, a genetic modification in a transcription factor sequence, inhibiting expression of the full-length transcription factor, can have downstream effects and inhibit expression of the expression of one or more gene products controlled by the transcription factor. Therefore, inhibition of expression can be predicted by changes in genomic or mRNA sequences. Therefore, mutations expected to result in inhibition of expression can be detected by known methods including sequencing of mRNA isolated from a tissue or cell population of interest. Inhibition of expression can be determined as the percent of cells in a population having a predetermined level of expression of a protein, i.e., a reduction of the percent or number of cells in a population expressing a protein of interest at at least a certain level. Inhibition of expression can also be assessed by determining a decrease in overall protein level, e.g., in a cell or tissue sample, e.g., a biopsy sample. In certain embodiments, inhibition of expression of a secreted protein can be assessed in a fluid sample, e.g., cell culture media or a body fluid. Proteins may be present in a body fluid, e.g., blood or urine, to permit analysis of protein level. In certain embodiments, protein level may be determined by protein activity or the level of a metabolic product, e.g., in urine or blood. In some embodiments. “inhibition of expression” may refer to some loss of expression of a particular gene product, for example a decrease in the amount of mRNA transcribed or a decrease in the amount of protein expressed by a population of cells. In some embodiments. “inhibition” may refer to some loss of expression of a particular gene product, for example a PD1 gene product at the cell surface. It is understood that the level of knockdown is relative to a starting level in the same type of subject sample. For example, routine monitoring of a protein level is more easily performed in a fluid sample from a subject, e.g., blood or urine, than in a tissue sample, e.g., a biopsy sample. It is understood that the level of knockdown is for the sample being assayed. Similarly, in animal studies where serial tissue samples may be obtained, e.g., liver tissue, the knockdown target may be expressed in other tissues. Therefore, the level of knockdown is not necessarily the level of knockdown systemically, but within the tissue, cell type, or fluid being sampled.

As used herein, a “genetic modification” is a change at the DNA level, e.g. induced by a CRISPR/Cas9 gRNA and Cas9 system. A genetic modification may comprise an insertion, deletion, or substitution (i.e., base sequence substitution, i.e., mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g., contiguous nucleotides. A genetic modification can be in a coding sequence, e.g., an exon sequence. A genetic modification can be at a splice site. i.e., sufficiently close to a splice acceptor site or a splice donor site to disrupt splicing. A genetic modification can include insertion of a nucleotide sequence not endogenous to the genomic locus, e.g., insertion of a coding sequence of a heterologous open reading frame or gene. As used herein, preferably a genetic modification prevents translation of a full-length protein having an amino acid sequence of the full-length protein prior to genetic modification of the genomic locus. Prevention of translation of a full-length protein or gene product includes prevention of translation of a protein or gene product of any length. Translation of a full-length protein can be prevented, for example, by a frameshift mutation that results in the generation of a premature stop codon or by generation of a nonsense mutation. Translation of a full-length protein can be prevented by disruption of splicing.

As used herein, a “heterologous coding sequence” refers to a coding sequence that has been introduced as an exogenous source within a cell (e.g., inserted at a genomic locus such as a safe harbor locus including a TCR gene locus). That is, the introduced coding sequence is heterologous with respect to at least its insertion site. A polypeptide expressed from such heterologous coding sequence gene is referred to as a “heterologous polypeptide.” The heterologous coding sequence can be naturally-occurring or engineered, and can be wild-type or a variant. The heterologous coding sequence may include nucleotide sequences other than the sequence that encodes the heterologous polypeptide (e.g., an internal ribosomal entry site). The heterologous coding sequence can be a coding sequence that occurs naturally in the genome, as a wild-type or a variant (e.g., mutant). For example, although the cell contains the coding sequence of interest (as a wild-type or as a variant), the same coding sequence or variant thereof can be introduced as an exogenous source for, e.g., expression at a locus that is highly expressed. The heterologous gcoding sequence can also be a coding sequence that is not naturally occurring in the genome, or that expresses a heterologous polypeptide that does not naturally occur in the genome. “Heterologous coding sequence”. “exogenous coding sequence”, and “transgene” are used interchangeably. In some embodiments, the heterologous coding sequence or transgene includes an exogenous nucleic acid sequence, e.g., a nucleic acid sequence is not endogenous to the recipient cell. In some embodiments, the heterologous coding sequence or transgene includes an exogenous nucleic acid sequence. e.g., a nucleic acid sequence that does not naturally occur in the recipient cell. For example, a heterologous coding sequence may be heterologous with respect to its insertion site and with respect to its recipient cell.

A “safe harbor” locus is a locus within the genome wherein a gene may be inserted without significant deleterious effects on the cell. Non-limiting examples of safe harbor loci that are targeted by nuclease(s) for use herein include AAVS1 (PPP1 R12C), TCR, B2M. In some embodiments, insertions at a locus or loci targeted for knockdown such as a TRC gene, e.g., TRAC gene, is advantageous for cells. Other suitable safe harbor loci are known in the art.

As used herein, “targeting receptor” refers to a receptor present on the surface of a cell, e.g., a T cell, to permit binding of the cell to a target site, e.g., a specific cell or tissue in an organism. Targeting receptors include, but are not limited to a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion of a protein.

As used herein, a “chimeric antigen receptor” refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837, the corresponding portions of the contents of each of which are incorporated herein by reference). A reversed universal CAR that promotes binding of an immune cell to a target cell through an adaptor molecule (see, e.g., WO2019238722, the contents of which are incorporated herein in their entirety) is also contemplated. CARs can be targeted to any antigen to which an antibody can be developed and are typically directed to molecules displayed on the surface of a cell or tissue to be targeted.

As used herein, “treatment” refers to any administration or application of a therapeutic for disease or disorder in a subject, and includes inhibiting the disease, arresting its development, relieving one or more symptoms of the disease, curing the disease, preventing one or more symptoms of the disease, or preventing reoccurrence of one or more symptoms of the disease. Treating an autoimmune or inflammatory response or disorder may comprise alleviating the inflammation associated with the specific disorder resulting in the alleviation of disease-specific symptoms. Treatment with the engineered T cells described herein may be used before, after, or in combination with additional therapeutic agents, e.g., the standard of care for the indication to be treated.

As used herein, “PD1” or “PD1” or “PD-1” refers to the nucleic acid sequence or protein sequence of “programmed cell death protein 1”: The human wild-type PD1 sequence is available at NCBI Gene ID: 5133 (worldwide web at ncbi.nlm.nih.gov/gene?cmd=retrieve&dopt-default&rn=1&list_uids=5133, in the version available on the date of filing the instant application): Ensembl: ENSG00000188389, chr2: 241849881-241858908.Synonyms for PD1 include PDCD1, CD279, SLEB2, hPD-1, and hSLE. PD1 is an immune-inhibitory receptor expressed in activated T cells, and plays a role in the regulation of T-cell functions, as well as the differentiation of CD4+ T cells into T regulatory cells. PD1 is expressed in many types of tumors. The PD1 gene contains 6 exons.

As used herein, “T cell receptor” or “TCR” refers to a receptor in a T cell. In general, a TCR is a heterodimer receptor molecule that contains two TCR polypeptide chains, α and β. α and β chain TCR polypeptides can complex with various CD3 molecules and elicit immune response(s), including inflammation and autoimmunity, after antigen binding. As used herein, a knockdown of TCR refers to a knockdown of any TCR gene in part or in whole, e.g., deletion of part of the TRBC1 gene, alone or in combination with knockdown of other TCR gene(s) in part or in whole.

“TRAC” is used to refer to the T cell receptor a chain. A human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

“TRBC” is used to refer to the T-cell receptor β-chain, e.g., TRBC1 and TRBC2. “TRBC1” and “TRBC2” refer to two homologous genes encoding the T-cell receptor β-chain, which are the gene products of the TRBC1 or TRBC2 genes.

A human wild-type TRBC1 sequence is available at NCBI Gene ID: 28639; Ensembl: ENSG00000211751. T-cell receptor Beta Constant, V_segment Translation Product, BV05S1J2.2. TCRBC1, and TCRB are gene synonyms for TRBC1.

A human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

A “T cell” plays a central role in the immune response following exposure to an antigen. T cells can be naturally occurring or non-natural, e.g., when T cells are formed by engineering, e.g., from a stem cell or by transdifferentiation, e.g., reprogramming a somatic cell. T cells can be distinguished from other lymphocytes by the presence of a T cell receptor on the cell surface. Included in this definition are conventional adaptive T cells, which include helper CD4+ T cells, cytotoxic CD8+ T cells, memory T cells, and regulatory CD4+ T cells, and innate-like T cells including natural killer T cells, mucosal associated invariant T cells, and gamma delta T cells. In some embodiments. T cells are CD4+. In some embodiments, T cells are CD3+/CD4+.

As used herein. “MHC” or “MHC protein” refers to a major histocompatibility complex molecule (or plural), and includes e.g., MHC class I molecules (e.g., HLA-A, HLA-B, and HLA-C in humans) and MHC class II molecules (e.g., HLA-DP, HLA-DQ, and HLA-DR in humans).

“CIITA” or “CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

“β2M” or “B2M,” as used herein, refers to nucleic acid sequence or protein sequence of “β-2 microglobulin”: the human gene has accession number NC_000015 (range 44711492 . . . 44718877), reference GRCh38.p13. The B2M protein is associated with MHC class I molecules as a heterodimer on the surface of nucleated cells and is required for MHC class I protein expression.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility. A alpha chain;” the human gene has accession number NC_000006.12 (29942532 . . . 29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: www.ebi.ac.uk/ipd/imgt/hla/. All alleles of HLA-A are encompassed by the terms “HLA-A” and “HLA-A gene.”

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

A “splice site,” as used herein, refers to the three nucleotides that make up an acceptor splice site or a donor splice site (defined below), or any other nucleotides known in the art that are part of a splice site. See e.g., Burset et al., Nucleic Acids Research 28(21):4364-4375 (2000) (describing canonical and non-canonical splice sites in mammalian genomes). The three nucleotides that make up an “acceptor splice site” are two conserved residues (e.g., AG in humans) at the 3′ of an intron and a boundary nucleotide (i.e., the first nucleotide of the exon 3′ of the AG). The “splice site boundary nucleotide” of an acceptor splice site is designated as “Y” in the diagram below and may also be referred to herein as the “acceptor splice site boundary nucleotide,” or “splice acceptor site boundary nucleotide.” The terms “acceptor splice site,” “splice acceptor site,” “acceptor splice sequence,” or “splice acceptor sequence” may be used interchangeably herein.

The three nucleotides that make up a “donor splice site” are two conserved residues (e.g., GT (gene) or GU (in RNA such as pre-mRNA) in human) at the 5′ end of an intron and a boundary nucleotide (i.e., the first nucleotide of the exon 5′ of the GT). The “splice site boundary nucleotide” of a donor splice site is designated as “X” in the diagram below and may also be referred to herein as the “donor splice site boundary nucleotide,” or “splice donor site boundary nucleotide.” The terms “donor splice site,” “splice donor site,” “donor splice sequence,” or “splice donor sequence” may be used interchangeably herein.

### II. Compositions

Compositions Comprising Guide RNA (gRNAs)

Provided herein are compositions useful for altering a DNA sequence, e.g., inducing a single-stranded (SSB) or double-stranded break (DSB), within a PD1 gene, e.g., using a guide RNA with an RNA-guided DNA binding agent (e.g., a CRISPR/Cas system). Guide sequences targeting a PD1 gene are shown in Table 1 at SEQ ID NOs: 1-88, as are the genomic coordinates that such guide RNA targets.

Each of the guide sequences shown in Table 1 at SEQ ID NOs: 1-88 may further comprise additional nucleotides to form a crRNA, e.g., with the following exemplary nucleotide sequence following the guide sequence at its 3′ end: GUUUUAGAGCUAUGCUGUUUUG (SEQ ID NO: 200) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGC (SEQ ID NO: 201) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 202) in 5′ to 3′ orientation.

In the case of a sgRNA, the guide sequences may be integrated into the following modified motif: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

In the case of a sgRNA, the guide sequences may further comprise a Spy Cas9 sgRNA sequence. An example of a Spy Cas9 sgRNA sequence is shown in the table below (SEQ ID NO: 201: GUUUUAGAGC UAGAAAUAGC AAGUUAAAAU AAGGCUAGUC CGUUAUCAAC UUGAAAAAGU GGCACCGAGU CGGUGC—“Exemplary SpyCas9 sgRNA-1”), included at the 3′ end of the guide sequence, and provided with the domains as shown in the table below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a Spy Cas9 sgRNA sequence, e.g., Exemplary SpyCas9 sgRNA-1. A gRNA, such as an sgRNA, may include modifications on the 5′ end of the guide sequence and/or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 201 (“Exemplary SpyCas9 sgRNA-1”) as an example, the Exemplary SpyCas9 sgRNA-1 further includes one or more of:
A. a shortened hairpin 1 region, or a substituted and optionally shortened hairpin A. 1 region, wherein
   1, at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a, any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2, the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 201) or
      b. one or more of positions H1-6 through H1-10 is substituted relative to Exemplary SpyCas9 sgRNA-1 (SEQ ID NO: 201); or
   3, the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 201); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 201); or
C. a substitution relative to Exemplary SpyCas9 sgRNA-1 (SEQ ID NO: 201) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 (SEQ ID NO: 201) with an upper stem region, wherein the upper stem modification comprises a modification to any one or more of US1-US12 in the upper stem region, wherein
   1, the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2, the modified nucleotide optionally includes a 2′-OMe modified nucleotide.

In certain embodiments, Exemplary SpyCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2″-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a substituted nucleotide, i.e., sequence substituted nucleotide, wherein the pyrimidine is substituted for a purine. In certain embodiments, when the pyrimidine forms a Watson-Crick base pair in the single guide, the Watson-Crick based nucleotide of the substituted pyrimidine nucleotide is substituted to maintain Watson-Crick base pairing.

Exemplary spyCas9 sgRNA-1 (SEQ ID NO: 201)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B3-B6 | | | | S7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| | |
|---|---|
| Nexus | H1-1 through H1-12 |

| 61 | 62 | 63 | 64 | 65 | 66 | 67 | 68 | 69 | 70 | 71 | 72 | 73 | 74 | 75 | 76 |
|---|---|---|---|---|---|---|---|---|---|---|---|---|---|---|---|
| G | G | C | A | C | C | G | A | G | U | C | G | G | U | G | C |

| | |
|---|---|
| N | H2-1 through H2-15 |

TABLE 1
PD1 guide sequences and chromosomal coordinates

| | | Guide | | |
|---|---|---|---|---|
| PD1 | | SEQ ID | | PD1 |
| Guide ID | Guide Sequence | NO | Genomic Coordinates (hg38) | NO |
| CR000833 | AGGCGCCCUGGCCAGUCGUC | 1 | chr2:241858805-241858825 | PD1-1 |
| CR000834 | GGCGCCCUGGCCAGUCGUCU | 2 | chr2:241858804-241858824 | PD1-2 |
| CR000836 | CACCGCCCAGACGACUGGCC | 3 | chr2:241858796-241858816 | PD1-3 |
| CR000837 | UGUAGCACCGCCCAGACGAC | 4 | chr2:241858791-241858811 | PD1-4 |
| CR000838 | CGUCUGGGCGGUGCUACAAC | 5 | chr2:241858789-241858809 | PD1-5 |
| CR000839 | GUCUGGGCGGUGCUACAACU | 6 | chr2:241858788-241858808 | PD1-6 |
| CR000840 | GGGCGGUGCUACAACUGGGC | 7 | chr2:241858784-241858804 | PD1-7 |
| CR000842 | CACCUACCUAAGAACCAUCC | 8 | chr2:241858755-241858775 | PD1-8 |
| CR000844 | CGGUCACCACGAGCAGGGCU | 9 | chr2:241852920-241852940 | PD1-9 |
| CR000845 | UCGGUCACCACGAGCAGGGC | 10 | chr2:241852919-241852939 | PD1-10 |
| CR000846 | GCCCUGCUCGUGGUGACCGA | 11 | chr2:241852919-241852939 | PD1-11 |
| CR000847 | CCCUUCGGUCACCACGAGCA | 12 | chr2:241852915-241852935 | PD1-12 |
| CR000849 | CCCCUUCGGUCACCACGAGC | 13 | chr2:241852914-241852934 | PD1-13 |
| CR000850 | CCUGCUCGUGGUGACCGAAG | 14 | chr2:241852917-241852937 | PD1-14 |
| CR000853 | CACGAAGCUCUCCGAUGUGU | 15 | chr2:241852864-241852884 | PD1-15 |
| CR000854 | CGGAGAGCUUCGUGCUAAAC | 16 | chr2:241852858-241852878 | PD1-16 |
| CR000856 | GCUUGUCCGUCUGGUUGCUG | 17 | chr2:241852821-241852841 | PD1-17 |
| CR000857 | AGCUUGUCCGUCUGGUUGCU | 18 | chr2:241852820-241852840 | PD1-18 |
| CR000858 | CAGCUUGUCCGUCUGGUUGC | 19 | chr2:241852819-241852839 | PD1-19 |
| CR000860 | AGGCGGCCAGCUUGUCCGUC | 20 | chr2:241852812-241852832 | PD1-20 |
| CR000868 | UGACACGGAAGCGGCAGUCC | 21 | chr2:241852764-241852784 | PD1-21 |
| CR000869 | GCAGUUGUGUGACACGGAAG | 22 | chr2:241852755-241852775 | PD1-22 |
| CR000870 | CGUGUCACACAACUGCCCAA | 23 | chr2:241852751-241852771 | PD1-23 |
| CR000871 | GUGUCACACAACUGCCCAAC | 24 | chr2:241852750-241852770 | PD1-24 |
| CR000872 | AUGUGGAAGUCACGCCCGUU | 25 | chr2:241852733-241852753 | PD1-25 |
| CR000874 | GCGUGACUUCCACAUGAGCG | 26 | chr2:241852728-241852748 | PD1-26 |
| CR000875 | ACUUCCACAUGAGCGUGGUC | 27 | chr2:241852723-241852743 | PD1-27 |
| CR000876 | CUUCCACAUGAGCGUGGUCA | 28 | chr2:241852722-241852742 | PD1-28 |
| CR000879 | AGGGCCCGGCGCAAUGACAG | 29 | chr2:241852703-241852723 | PD1-29 |
| CR000880 | GGUGCCGCUGUCAUUGCGCC | 30 | chr2:241852696-241852716 | PD1-30 |
| CR000885 | GAUCUGCGCCUUGGGGGCCA | 31 | chr2:241852654-241852674 | PD1-31 |
| CR000889 | CUCUCUUUGAUCUGCGCCUU | 32 | chr2:241852646-241852666 | PD1-32 |
| CR000890 | GCUCUCUUUGAUCUGCGCCU | 33 | chr2:241852645-241852665 | PD1-33 |
| CR000894 | AGGGUUUGGAACUGGCCGGC | 34 | chr2:241852283-241852303 | PD1-34 |
| CR000901 | ACCCUGGUGGUUGGUGUCGU | 35 | chr2:241852268-241852288 | PD1-35 |
| CR000902 | GCCCACGACACCAACCACCA | 36 | chr2:241852264-241852284 | PD1-36 |
| CR000903 | CGCCCACGACACCAACCACC | 37 | chr2:241852263-241852283 | PD1-37 |
| CR000904 | CUGGUGGUUGGUGUCGUGGG | 38 | chr2:241852265-241852285 | PD1-38 |
| CR000908 | UCCUGGCCGUCAUCUGCUCC | 39 | chr2:241852210-241852230 | PD1-39 |
| CR000911 | GACGUUACCUCGUGCGGCCC | 40 | chr2:241852189-241852209 | PD1-40 |
| CR000912 | UGACGUUACCUCGUGCGGCC | 41 | chr2:241852188-241852208 | PD1-41 |
| CR000913 | UGGGAUGACGUUACCUCGUG | 42 | chr2:241852183-241852203 | PD1-42 |
| CR002588 | CGACUGGCCAGGGCGCCUGU | 43 | chr2:241858807-241858827 | PD1-43 |
| CR007233 | UGCGGUACCAGUUUAGCACG | 44 | chr2:241852848-241852868 | PD1-44 |
| CR007234 | AGGUGCCCAUUCCGCUAGGA | 45 | chr2:241851146-241851166 | PD1-45 |
| CR007235 | GGCCCCCAAGGCGCAGAUCA | 46 | chr2:241852653-241852673 | PD1-46 |
| CR007236 | UGGAUUUCCAGUGGCGAGAG | 47 | chr2:241851228-241851248 | PD1-47 |
| CR007237 | ACCUCGUGCGGCCCGGGAGC | 48 | chr2:241852195-241852215 | PD1-48 |
| CR007238 | AUCAAAGAGAGCCUGCGGGC | 49 | chr2:241852637-241852657 | PD1-49 |
| CR007239 | CCGCCGACCCCACCUACCUA | 50 | chr2:241858745-241858765 | PD1-50 |
| CR007240 | GUGGCAUACUCCGUCUGCUC | 51 | chr2:241851175-241851195 | PD1-51 |
| CR007241 | CUACAACUGGGCUGGCGGCC | 52 | chr2:241858776-241858796 | PD1-52 |
| CR007242 | UGGCCAAGGAAGCCGGUCAG | 53 | chr2:241851041-241851061 | PD1-53 |
| CR007243 | UGCUGGGGCUCAUGCGGUAC | 54 | chr2:241852836-241852856 | PD1-54 |
| CR007244 | GCUACAACUGGGCUGGCGGC | 55 | chr2:241858777-241858797 | PD1-55 |
| CR007245 | CCAGUGGCGAGAGAAGACCC | 56 | chr2:241851221-241851241 | PD1-56 |
| CR007246 | UGCGGCCCGGGAGCAGAUGA | 57 | chr2:241852201-241852221 | PD1-57 |
| CR007247 | CGUUGGGCAGUUGUGUGACA | 58 | chr2:241852749-241852769 | PD1-58 |
| CR007248 | GCCACCAUUGUCUUUCCUAG | 59 | chr2:241851160-241851180 | PD1-59 |
| CR007249 | CCCCCAAGGCGCAGAUCAAA | 60 | chr2:241852651-241852671 | PD1-60 |
| CR007250 | GUGCUAAACUGGUACCGCAU | 61 | chr2:241852847-241852867 | PD1-61 |
| CR007251 | CGAAGCUCUCCGAUGUGUUG | 62 | chr2:241852866-241852886 | PD1-62 |
| CR007252 | AACGGGCGUGACUUCCACAU | 63 | chr2:241852733-241852753 | PD1-63 |
| CR007253 | UUGUCCCCUUCGGUCACCAC | 64 | chr2:241852910-241852930 | PD1-64 |
| CR007254 | GCAGCUUCUCCAACACAUCG | 65 | chr2:241852876-241852896 | PD1-65 |
| CR007255 | GUGGCCAAGGAAGCCGGUCA | 66 | chr2:241851040-241851060 | PD1-66 |
| CR007256 | CCCAAGGCGCAGAUCAAAGA | 67 | chr2:241852649-241852669 | PD1-67 |
| CR007257 | AGCCCCUGCGGGCGGGGGAU | 68 | chr2:241851125-241851145 | PD1-68 |
| CR007258 | CAUCUGCUCCCGGGCCGCAC | 69 | chr2:241852200-241852220 | PD1-69 |
| CR007259 | AGCUUCUCCAACACAUCGGA | 70 | chr2:241852874-241852894 | PD1-70 |
| CR007260 | AGUGGCGAGAGAAGACCCCG | 71 | chr2:241851219-241851239 | PD1-71 |
| CR007261 | ACAAGCUGGCCGCCUUCCCC | 72 | chr2:241852807-241852827 | PD1-72 |
| CR007262 | CACUCCGAGGGCCGUCAGCU | 73 | chr2:241851104-241851124 | PD1-73 |
| CR007263 | CGUUACCUCGUGCGGCCCGG | 74 | chr2:241852191-241852211 | PD1-74 |
| CR007264 | ACGAAGCUCUCCGAUGUGUU | 75 | chr2:241852865-241852885 | PD1-75 |
| CR007265 | CUGCUCGUGGUGACCGAAGG | 76 | chr2:241852916-241852936 | PD1-76 |
| CR007266 | CCGGGAGCAGAUGACGGCCA | 77 | chr2:241852207-241852227 | PD1-77 |
| CR007267 | ACGUUACCUCGUGCGGCCCG | 78 | chr2:241852190-241852210 | PD1-78 |
| CR007268 | AUUGUCUUUCCUAGCGGAAU | 79 | chr2:241851154-241851174 | PD1-79 |
| CR007269 | UCAGUGGCUGGGCACUCCGA | 80 | chr2:241851092-241851112 | PD1-80 |
| CR007270 | UGGCAUACUCCGUCUGCUCA | 81 | chr2:241851176-241851196 | PD1-81 |
| CR007271 | ACCUCAUCCCCCGCCCGCAG | 82 | chr2:241851130-241851150 | PD1-82 |
| CR007272 | ACCGCCCAGACGACUGGCCA | 83 | chr2:241858797-241858817 | PD1-83 |
| CR007273 | GGGCUCAUGCGGUACCAGUU | 84 | chr2:241852841-241852861 | PD1-84 |
| CR007274 | ACGACUGGCCAGGGCGCCUG | 85 | chr2:241858806-241858826 | PD1-85 |
| CR007275 | CCGGGGUCUUCUCUCGCCAC | 86 | chr2:241851218-241851238 | PD1-86 |
| CR007276 | GCAGCCUGGUGCUGCUAGUC | 87 | chr2:241852234-241852254 | PD1-87 |
| CR007277 | AGCCGGCCAGUUCCAAACCC | 88 | chr2:241852284-241852304 | PD1-88 |

TABLE 2
Exemplary sgRNA sequences targeting PD1

| Guide | |
|---|---|
| ID | sgRNA Sequence |
| G015072 | CGUGUCACACAACUGCCCAAGUUUUAGAGC |
| | UAGAAAUAGCAAGUUAAAAUAAGGCUAGUC |
| | CGUUAUCAACUUGAAAAAGUGGCACCGAGU |
| | CGGUGCUUUU (SEQ ID NO: 101) |
| G015073 | CCCUUCGGUCACCACGAGCAGUUUUAGAGC |
| | UAGAAAUAGCAAGUUAAAAUAAGGCUAGUC |
| | CGUUAUCAACUUGAAAAAGUGGCACCGAGU |
| | CGGUGCUUUU (SEQ ID NO: 102) |
| G015074 | GCAGUUGUGUGACACGGAAGGUUUUAGAGC |
| | UAGAAAUAGCAAGUUAAAAUAAGGCUAGUC |
| | CGUUAUCAACUUGAAAAAGUGGCACCGAGU |
| | CGGUGCUUUU (SEQ ID NO: 103) |
| G015075 | CGACUGGCCAGGGCGCCUGUGUUUUAGAGC |
| | UAGAAAUAGCAAGUUAAAAUAAGGCUAGUC |
| | CGUUAUCAACUUGAAAAAGUGGCACCGAGU |
| | CGGUGCUUUU (SEQ ID NO: 104) |
| G015076 | CGUCUGGGCGGUGCUACAACGUUUUAGAGC |
| | UAGAAAUAGCAAGUUAAAAUAAGGCUAGUC |
| | CGUUAUCAACUUGAAAAAGUGGCACCGAGU |
| | CGGUGCUUUU (SEQ ID NO: 105) |
| G015077 | GCCCUGCUCGUGGUGACCGAGUUUUAGAGC |
| | UAGAAAUAGCAAGUUAAAAUAAGGCUAGUC |
| | CGUUAUCAACUUGAAAAAGUGGCACCGAGU |
| | CGGUGCUUUU (SEQ ID NO: 106) |

In some embodiments, a composition comprising one or more guide RNAs (gRNA) comprising guide sequences that direct an RNA-guided DNA binding agent, which can be a nuclease (e.g., a Cas nuclease such as Cas9), to a target DNA sequence in PD1 are provided. In some embodiments, an engineered cell comprising a genetic modification in a human PD1 sequence within genomic coordinates of chr2: 241849881-241858908 is provided. In some embodiments, an engineered cell comprising a genetic modification in a human PD1 sequence is provided, wherein the genetic modification comprises a modification of at least one nucleotide within the genomic coordinates corresponding to PD1 guide sequence selected from PD1-1 through PD1-88. In some embodiments, an engineered cell comprising a genetic modification in a human PD1 sequence is provided, wherein the genetic modification comprises a modification of at least one nucleotide within the genomic coordinates selected from Table 3:

TABLE 3
Select PD1 guide sequences and chromosomal coordinates

| PD1 NO. | Genomic Coordinates (hg38) |
|---|---|
| PD1-29 | chr2: 241852703-241852723 |
| PD1-43 | chr2: 241858807-241858827 |
| PD1-5 | chr2: 241858789-241858809 |
| PD1-6 | chr2: 241858788-241858808 |
| PD1-8 | chr2: 241858755-241858775 |
| PD1-11 | chr2: 241852919-241852939 |
| PD1-12 | chr2: 241852915-241852935 |
| PD1-22 | chr2: 241852755-241852775 |
| PD1-23 | chr2: 241852751-241852771 |
| PD1-24 | chr2: 241852750-241852770 |
| PD1-36 | chr2: 241852264-241852284 |
| PD1-57 | chr2: 241852201-241852221. |
| PD1-58 | chr2: 241852749-241852769 |
| PD1-17 | chr2: 241852821-241852841 |
| PD1-38 | chr2: 241852265-241852285 |
| PD1-56 | chr2: 241851221-241851241 |
| PD1-41 | chr2: 241852188-241852208 |

In some embodiments comprising a gRNA, the gRNA may comprise a crRNA comprising a guide sequence shown in Table 1 as a guide sequence. In some embodiments, the gRNA comprises a guide sequence shown in Table 1, e.g. as an sgRNA. In some embodiments, the gRNA may comprise a guide sequence selected from SEQ ID NOs: 1-88; SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; or SEQ ID NOs: 6 and 29; or SEQ ID NOs: 6, 23, 29, 41, and 57; or SEQ ID NOs: 6, 29, and 57; or SEQ ID NO: 43. In some embodiments, the gRNA may comprise a guide sequence selected from SEQ ID NOs: 5, 6, 8, 11, 12, 17, 22, 23, 24, 29, 36, 38, 41, 43, 56, 57 and 58. In some embodiments, the gRNA may comprise a guide sequence selected from SEQ ID NOs: 5, 6, 8, 11, 12, 22, 23, 24, 29, 36, 43, and 57. In some embodiments, the gRNA may comprise a guide sequence selected from SEQ ID NOs:5, 11, 12, 22, 23, and 43. In some embodiments, the gRNA may comprise a guide sequence selected from SEQ ID NOs: 6, 8, 23, and 29. In some embodiments, the gRNA may comprise a guide sequence selected from SEQ ID NOs: 6 and 29. In some embodiments, the gRNA may comprise a guide sequence selected from SEQ ID NOs: 6, 23, 29, 41, and 57. In some embodiments, the gRNA may comprise a guide sequence selected from SEQ ID NOs: 6, 29, and 57. In some embodiments, the gRNA may comprise a guide sequence of SEQ ID NO: 43. The gRNA may comprise a guide sequence comprising 17, 18, 19, or 20 contiguous nucleotides of a guide sequence shown in Table 1. In some embodiments, the gRNA comprises a sequence with at least 75%, 80%, 85%, 90%, or 95%, or 100% identity to at least 17, 18, 19, or 20 contiguous nucleotides of a guide sequence shown in Table 1. In some embodiments, the gRNA comprises a sequence with at least 75%, 80%, 85%, 90%, or 95%, or 100% identity to a guide sequence shown in Table 1. The gRNA may further comprise a trRNA. In each embodiment described herein, the gRNA may comprise a crRNA and trRNA associated as a single RNA (sgRNA) or on separate RNAs (dgRNA). In the context of sgRNAs, the crRNA and trRNA components may be covalently linked, e.g., via a phosphodiester bond or other covalent bond.

In each embodiment described herein, the guide RNA may comprise two RNA molecules as a “dual guide RNA” or “dgRNA.” The dgRNA comprises a first RNA molecule comprising a crRNA comprising, e.g., a guide sequence shown in Table 1, and a second RNA molecule comprising a trRNA. The first and second RNA molecules may not be covalently linked, but may form an RNA duplex via the base pairing between portions of the crRNA and the trRNA.

In each embodiment described herein, the guide RNA may comprise a single RNA molecule as a “single guide RNA” or “sgRNA”. The sgRNA may comprise a crRNA (or a portion thereof) comprising a guide sequence shown in Table 1, or a guide sequence selected from SEQ ID NOs: 1-88; SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43, covalently linked to a trRNA. The sgRNA may comprise 17, 18, 19, or 20 contiguous nucleotides of a guide sequence shown in Table 1, or a guide sequence selected from SEQ ID NOs: 1-88; SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; SEQ ID NOs: 6, 29, and 57; or SEQ ID NO: 43. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one of SEQ ID NOs: 1-88, or SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43 is provided.

In some embodiments, a composition comprising one or more sgRNAs comprising any one of SEQ ID NOs: 101-106 is provided.

In one aspect, a composition comprising a gRNA that comprises a guide sequence that is at least 90% or 95% identical to any of the nucleic acids of SEQ ID NOs: 1-88 is provided. In some embodiments, a composition comprising a gRNA that comprises a guide sequence that is at least 90% or 95% identical to any of SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43 is provided.

In some embodiments, a composition is provided comprising at least one, e.g., at least two gRNA's, comprising guide sequences selected from any one or two or more of the guide sequences of SEQ ID NOs: 1-88; or SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; or SEQ ID NOs: 6 and 29; or SEQ ID NOs: 6, 23, 29, 41, and 57; or SEQ ID NOs: 6, 29, and 57; or SEQ ID NO: 43. In some embodiments, the composition comprises at least two gRNA's that each comprise a guide sequence that is at least 90% or 95% identical to any of the nucleic acids of SEQ ID NOs: 1-88; or SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43.

The guide RNA compositions provided herein are designed to recognize (e.g., hybridize to) a target sequence in a PD1 gene. For example, the PD1 target sequence may be recognized and cleaved by a provided Cas cleavase comprising a guide RNA. In some embodiments, an RNA-guided DNA binding agent, such as a Cas cleavase, may be directed by a guide RNA to a target sequence of a PD1 gene, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within a PD1 gene.

Without being bound by any particular theory, mutations (e.g., frameshift mutations resulting from indels, i.e., insertions or deletions, occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary or having complementarity to a target sequence within PD1 is used to direct the RNA-guided DNA binding agent to a particular location in the appropriate PD1 gene. In some embodiments, gRNAs are designed to have guide sequences that are complementary or have complementarity to target sequences in exon 1, exon 2, exon 3, exon 4, exon 5, or exon 6 of PD1.

In some embodiments, the guide sequence is at least 90%, 95%, or 100% identical to the reverse complement of a target sequence present in a human PD1 gene. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, or 95%; or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G. C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification): (ii) alteration, e.g., replacement, of a constituent of the ribose sugar, e.g., of the 2′ hydroxyl on the ribose sugar (an exemplary sugar modification): (iii) wholesale replacement of the phosphate moiety with “dephospho” linkers (an exemplary backbone modification); (iv) modification or replacement of a naturally occurring nucleobase, including with a non-canonical nucleobase (an exemplary base modification): (v) replacement or modification of the ribose-phosphate backbone (an exemplary backbone modification): (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar or backbone modification); and (vii) modification or replacement of the sugar (an exemplary sugar modification).

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e, at sugar modification. For example, the 2′ hydroxyl group (OH) can be modified, e.g. replaced with a number of different “Oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0) to 20 (e.g., from 0) to 4, from 0 to 8, from 0 to 10, from 0) to 16, from 1 to 4, from 1 to 8, from 1 to 10, from 1 to 16, from 1 to 20), from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e, deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the entire sgRNA may be chemically modified. Certain embodiments comprise a 5′ end modification. Certain embodiments comprise a 3′ end modification. Additional embodiments comprise a 5′ end modification and a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety.

In some embodiments, the sgRNA comprises any of the modification patterns shown herein, where N is any natural or non-natural nucleotide, and wherein the totality of the N's comprise a PD1 guide sequence as described herein in Table 1. In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise a PD1 guide sequence as described in Table 1, for example. For example, where the N's are replaced with any of the guide sequences disclosed herein in Table 1, optionally wherein the N's are replaced with SEQ ID NOs: 1-88; or SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43.

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

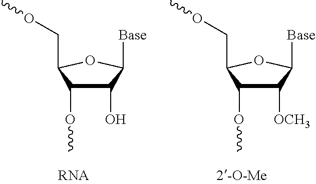

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

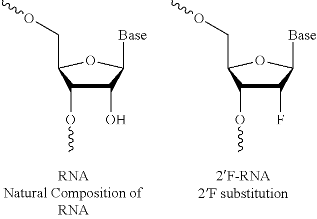

Phosphorothioate (PS) linkage or bond refers to a bond where a sulfur is substituted for one non-bridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S— into a non-bridging phosphate oxygen, generating a PS bond in lieu of a phosphodiester bond:

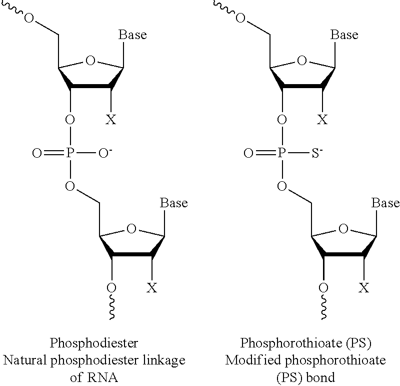

Abasic nucleotides refer to those which lack nitrogenous bases. The figure below depicts an oligonucleotide with an abasic (also known as apurinic) site that lacks a base:

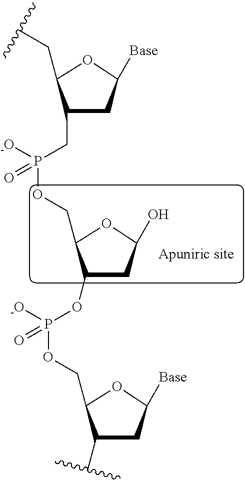

Inverted bases refer to those with linkages that are inverted from the normal 5′ to 3′ linkage (i.e., either a 5′ to 5′ linkage or a 3′ to 3′ linkage). For example:

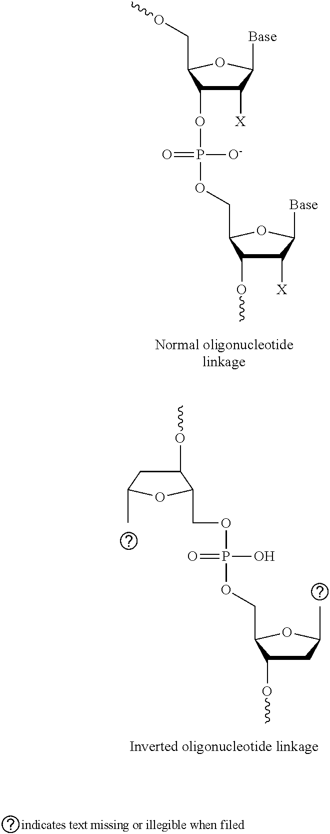

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where N is any natural or non-natural nucleotide, and where the totality of the N's comprise a guide sequence that directs a nuclease to a target sequence in PD1, e.g., the genomic coordinates shown in Table 1.

In some embodiments, the guide RNA comprises a sgRNA comprising any one of the guide sequences of SEQ ID NOs: 1-88 and a conserved portion of an sgRNA, for example, the conserved portion of sgRNA shown as Exemplary SpyCas9 sgRNA-1 or the conserved portions of the gRNAs shown in Table 2 and throughout the specification. In some embodiments, the guide RNA comprises a sgRNA comprising any one of the guide sequences of SEQ ID NOs: 1-88 and the nucleotides of GUUUUAGAGCUAGAAAUAGCAAGUUAAAAUAAGGCUAGUCCGUUAUCAACUU GAAAAAGUGGCACCGAGUCGGUGCUUUU (SEQ ID NO: 202), wherein the nucleotides are on the 3′ end of the guide sequence, and wherein the sgRNA may be modified as shown herein or in the sequence mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300). In some embodiments, the sgRNA comprises Exemplary SpyCas9 sgRNA-1 or the modified versions thereof provided herein, or a version as provided in the TABLE 3B below, where the totality of the N's comprise a guide sequence that directs a nuclease to a target sequence. Each N is independently modified or unmodified. In certain embodiments, in the absence of an indication of a modification, the nucleotide is an unmodified RNA nucleotide residue, i.e., a ribose sugar and a phosphodiester backbone.

TABLE 3B
exemplary sgRNA sequences (modified and unmodified
versions).

| Guide Scaffold | sgRNA unmodified | |
|---|---|---|
| (unmodified/modified) | sequence | sgRNA modified sequence |
| 81/181 | (N)₂₀GUUUUAGAGCUAG | mN*mN*mN*(N)₁₇GUUU |
| | AAAUAGCAAGUUAAAA | UAGAmGmCmUmAmGm |
| | UAAGGCUAGUCCGUUA | AmAmAmUmAmGmCAA |
| | UCACGAAAGGGCACCG | GUUAAAAUAAGGCUAG |
| | AGUCGGUGC (SEQ ID | UCCGUUAUCACGAAAG |
| | NO: 401) | GGCACCGAGUCGG*mU |
| | | *mG*mC (SEQ ID NO: |
| | | 402) |
| 94/194 | (N)₂₀GUUUUAGAGCUAG | mN*mN*mN*(N)₁₇GUUU |
| | AAAUAGCAAGUUAAAA | UAGAmGmCmUmAmGm |
| | UAAGGCUAGUCCGUUA | AmAmAmUmAmGmCAA |
| | UCAACUUGGCACCGAG | GUUAAAAUAAGGCUAG |
| | UCGGUGC (SEQ ID NO: | UCCGUUAUCAACUUGG |
| | 403) | CACCGAGUCGG*mU*m |
| | | G*mC (SEQ ID NO: 404) |
| 95/195 | (N)₂₀GUUUUAGAGCUAG | mN*mN*mN*(N)₁₇GUUU |
| | AAAUAGCAAGUUAAAA | UAGAmGmCmUmAmGm |
| | UAAGGCUAGUCCGUUA | AmAmAmUmAmGmCAA |
| | UCAACUUGGCACCGAG | GUUAAAAUAAGGCUAG |
| | UCGGUGC (SEQ ID NO: | UCCGUUAUCAACUUGG |
| | 405) | CACCGAGUCGG*mU*m |
| | | G*mC (SEQ ID NO: 406) |
| 871/971 | (N)₂₀GUUUUAGAGCUAG | mN*mN*mN*(N)₁₇mGUU |
| | AAAUAGCAAGUUAAAA | UfUAGmAmGmCmUmAm |
| | UAAGGCUAGUCCGUUA | GmAmAmAmUmAmGmC |
| | UCACGAAAGGGCACCG | mAmAGUfUmAfAmAfAm |
| | AGUCGGUGC (SEQ ID | UAmAmGmGmCmUmAG |
| | NO: 407) | UmCmCGUfUAmUmCAm |
| | | CmGmAmAmAmGmGmG |
| | | mCmAmCmCmGmAmGm |
| | | UmCmGmG*mU*mG*mC |
| | | (SEQ ID NO: 408) |
| 872/972 | (N)₂₀GUUUUAGAGCUAG | mN*mN*mN*(N)₁₇GUUU |
| | AAAUAGCAAGUUAAAA | UAGAmGmCmUmAmGm |
| | UAAGGCUAGUCCGUUA | AmAmAmUmAmGmCAA |
| | UCACGAAAGGGCACCG | GUUAAAAUAAGGCUAG |
| | AGUCGGUGC (SEQ ID | UCCGUUAUCACGAAAG |
| | NO: 409) | GGCACCGAGUCGG*mU |
| | | *mG*mC (SEQ ID NO: |
| | | 410) |

As noted above, in some embodiments, a composition or formulation disclosed herein comprises an mRNA comprising an open reading frame (ORF) encoding an RNA-guided DNA binding agent, such as a Cas nuclease, e.g. Cas9 nuclease, as described herein. In some embodiments, an mRNA comprising an ORF encoding an RNA-guided DNA binding agent, such as a Cas nuclease, e.g. Cas9 nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the mRNA and/or modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine. N1-methyl-pseudouridine. 5-methoxyuridine. 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33): 12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

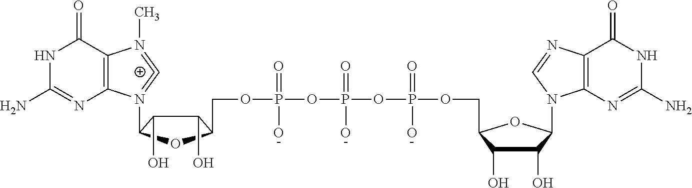

CleanCap™ AG (m7G(5′)ppp(5′)(2′OMeA)pG: TriLink Biotechnologies Cat. No. N-7113) or CleanCap™ GG (m7G(5′)ppp(5′)(2′OMeG)pG: TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

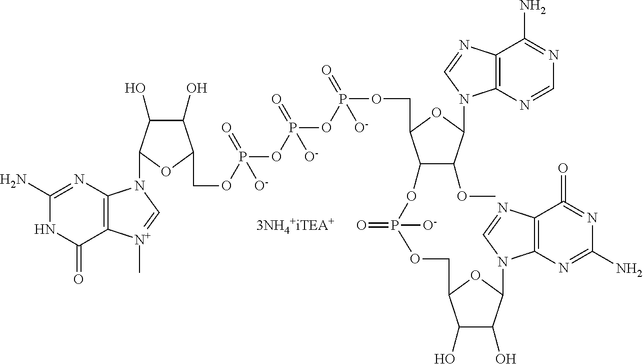

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its DI subunit, and guanine methyltransferase, provided by its D12 subunit. As such, it can add a 7-methylguanine to an RNA, so as to give Cap0, in the presence of S-adenosyl methionine and GTP. See, e.g., Guo, P, and Moss, B. (1990) Proc. Natl. Acad. Sci. USA 87, 4023-4027; Mao, X, and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

### Ribonucleoprotein Complex

In some embodiments, a composition is encompassed comprising one or more gRNAs comprising one or more guide sequences from Table 1 or one or more sgRNAs from Table 2 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes. S. aureus, and other prokaryotes (see, e.g., the list in the next paragraph), and modified (e.g., engineered or mutant) versions thereof. See, e.g., US20160312198; US 20160312199. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015): Shmakov et al., MOLECULAR CELL. 60:385-397 (2015).

Non-limiting exemplary species that the Cas nuclease can be derived from include Streptococcus pyogenes, Streptococcus thermophilus. Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis. Campylobacter jejuni. Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans. Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile. Finegoldia magna, Natranaerobius thermophilus, Pelotomaculum thermopropionicum, Acidithiobacillus caldus, Acidithiobacillus ferrooxidans, Allochromatium vinosum. Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens. Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A. H840A. N863A. H983A, and D986A (based on the S. pyogenes Cas) protein). See, e.g., Zetsche et al. (2015). Further exemplary amino acid substitutions include D917A. E1006A, and D1255A (based on the Francisella novicida U112 Cpf1 (FnCpf1) sequence (UniProtKB—AOQ7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 20140186958; US 20150166980.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS. PKKKRKV (SEQ ID NO: 89) or PKKKRRV (SEQ ID NO: 90). In some embodiments, the NLS may be a bipartite sequence, such as the NLS of nucleoplasmin. KRPAATKKAGQAKKKK (SEQ ID NO: 91). In a specific embodiment, a single PKKKRKV (SEQ ID NO: 92) NLS may be linked at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12). Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP. GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine. Venus, YPet, PhiYFP, ZsYellow 1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1. DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AUI, AU5, E, ECS, E2, FLAG, HA, nus, Softag 1, Softag 3, Strep, SBP, Glu-Glu, HSV, KT3, S, S1, T7, V5, VSV-G, 6xHis, 8xHis, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” (′el/152:1173-83 (2013): Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering.” Nat. Biotechnol. 31:833-8 (2013): Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase.

Determination of Efficacy of gRNAs

In some embodiments, the efficacy of a gRNA is determined when delivered or expressed together with other components forming an RNP. In some embodiments, the gRNA is expressed together with an RNA-guided DNA binding agent, such as a Cas protein, e.g. Cas9. In some embodiments, the gRNA is delivered to or expressed in a cell line that already stably expresses an RNA-guided DNA nuclease, such as a Cas nuclease or nickase. e.g. Cas9 nuclease or nickase. In some embodiments the gRNA is delivered to a cell as part of a RNP. In some embodiments, the gRNA is delivered to a cell along with a mRNA encoding an RNA-guided DNA nuclease, such as a Cas nuclease or nickase, e.g. Cas9 nuclease or nickase.

As described herein, use of an RNA-guided DNA nuclease and a guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein. In some embodiments, the efficacy of particular gRNAs is determined based on in vitro models. In some embodiments, the in vitro model is HEK293 cells stably expressing Cas9 (HEK293_Cas9). In some embodiments the in vitro model is a peripheral blood mononuclear cell (PBMC). In some embodiments, the in vitro model is a T cell, such as primary human T cells. With respect to using primary cells, commercially available primary cells can be used to provide greater consistency between experiments. In some embodiments, the number of off-target sites at which a deletion or insertion occurs in an in vitro model (e.g., in T cell) is determined, e.g., by analyzing genomic DNA from transfected cells in vitro with Cas9 mRNA and the guide RNA. In some embodiments, such a determination comprises analyzing genomic DNA from the cells transfected in vitro with Cas9 mRNA, the guide RNA, and a donor oligonucleotide. Exemplary procedures for such determinations are provided in the working examples in which HEK293 cells, PBMCs, and human CD3′ T cells are used.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of a guide RNA is measured by percent indels or percent genetic modifications of PD1. In some embodiments, the efficacy of a guide RNA is measured by percent indels or percent genetic modifications at a PD1 locus. In some embodiments, the efficacy of a guide RNA is measured by percent indels or percent genetic modifications of PD1 at genomic coordinates of Table 1. In some embodiments, the percent editing of PD1 is compared to the percent indels or genetic modifications necessary to achieve knockdown of the PD1 protein products. In some embodiments, the efficacy of a guide RNA is measured by reduced or eliminated expression of PD1 protein. In embodiments, said reduced or eliminated expression of PD1 protein is as measured by flow cytometry, e.g., as described herein.

In some embodiments, the PD1 protein expression is reduced or eliminated in a population of cells using the methods and compositions disclosed herein. In some embodiments, the population of cells is at least 55%, 60%, 65%, 70%, 80%, 90%, 91%, 92%, 93%, 94%, 95%, 96%, 97%, 98%, or 99% PD1 negative as measured by flow cytometry relative to a population of unmodified cells.

An “unmodified cell” (or “unmodified cells”) refers to a control cell (or cells) of the same type of cell in an experiment or test, wherein the “unmodified” control cell has not been contacted with a PD1 guide. Therefore, an unmodified cell (or cells) may be a cell that has not been contacted with a guide RNA, or a cell that has been contacted with a guide RNA that does not target PD1.

In some embodiments, the efficacy of a guide RNA is measured by the number or frequency of indels or genetic modifications at off-target sequences within the genome of the target cell type, such as a T cell. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., a T cell), or which produce a frequency of off-target indel formation of <5% in a cell population or relative to the frequency of indel creation at the target site. In some embodiments, the disclosure provides guide RNAs which do not exhibit any off target indel formation in the target cell type (e.g., T cell). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and insertion or homology directed repair (HDR) events in target DNA utilize linear amplification with a tagged primer and isolating the tagged amplification products (herein after referred to as “LAM-PCR,” or “Linear Amplification (LA)” method). In some embodiments, the efficacy of a guide RNA is measured by the levels of functional protein complexes comprising the expressed protein product of the gene. In some embodiments, the efficacy of a guide RNA is measured by flow cytometric analysis of TCR expression by which the live population of edited cells is analyzed for loss of the TCR.

### T Cell Receptors (TCR)

In some embodiments, the engineered cells or population of cells comprising a genetic modification, e.g., of an endogenous nucleic acid sequence encoding PD1, further comprise a modification, e.g., knockdown, of an endogenous nucleic acid sequence encoding TCR gene sequence(s), e.g., TRAC or TRBC.

In some embodiments, the engineered cells or population of cells comprising a genetic modification, e.g., knockdown, of an endogenous nucleic acid sequence encoding PD1 and insertion into the cell of heterologous sequence(s) encoding a targeting receptor, further comprise a modification, e.g., knockdown, of an endogenous nucleic acid sequence encoding TCR gene sequence(s), e.g., TRAC or TRBC.

Generally, a TCR is a heterodimer receptor molecule that contains two TCR polypeptide chains, α and β. Suitable a and ß genomic sequences or loci to target for knockdown are known in the art. In some embodiments, the engineered T cells comprise a modification, e.g., knockdown, of a TCR α-chain gene sequence, e.g., TRAC. See, e.g., NCBI Gene ID: 28755; Ensembl: ENSG00000277734 (T-cell receptor Alpha Constant), US 2018/0362975, and WO2020081613.

In some embodiments, the engineered cells or population of cells comprise a genetic modification of an endogenous nucleic acid sequence encoding PD1, a genetic modification, e.g., knockdown, of an endogenous nucleic acid sequence encoding TCR gene sequence(s), e.g., TRAC or TRBC; and modification, e.g., knockdown of an MHC class I gene, e.g., B2M or HLA-A. In some embodiments, an MHC class I gene is an HLA-B gene or an HLA-C gene.

In some embodiments, the engineered cells or population of cells comprise a genetic modification of an endogenous nucleic acid sequence encoding PD1 and a genetic modification, e.g., knockdown, of an endogenous nucleic acid sequence encoding TCR gene sequence(s), e.g., TRAC or TRBC; and a genetic modification, e.g., knockdown of an MHC class II gene, e.g., CIITA.

In some embodiments, the engineered cells or population of cells comprise a modification of an endogenous nucleic acid sequence encoding PD1, a genetic modification, e.g., knockdown, of an endogenous nucleic acid sequence encoding TCR gene sequence(s), e.g., TRAC or TRBC; and a genetic modification, e.g. knockdown of a checkpoint inhibitor gene, e.g., TIM3, 2B4, or LAG3.

In some embodiments, the engineered cells or population of cells comprise a genetic modification of a PD1 gene as assessed by sequencing, e.g., NGS, wherein at least 50%, 55%, 60%, 65%, preferably at least 70%, 75%, 80%, 85%, 90%, 95%, 96%, 97%, 98%, or 99% of cells comprise an insertion, deletion, or substitution in the endogenous PD1 sequence. In some embodiments, at least 50% of cells in the population comprise a modification selected from an insertion, a deletion, and a substitution in the endogenous PD1 sequence. In some embodiments, at least 55% of cells in the population comprise a modification In some embodiments, at least 60% of cells in the population comprise a modification selected from an insertion, a deletion, and a substitution in the endogenous PD1 sequence. In some embodiments, at least 65% of cells in the population comprise a modification selected from an insertion, a deletion, and a substitution in the endogenous PD1 sequence, on selected from an insertion, a deletion, and a substitution in the endogenous PD1 sequence. In some embodiments, at least 70% of cells in the population comprise a modification selected from an insertion, a deletion, and a substitution in the endogenous PD1 sequence. In some embodiments, at least 75% of cells in the population comprise a modification selected from an insertion, a deletion, and a substitution in the endogenous PD1 sequence. In some embodiments, at least 85% of cells in the population comprise a modification selected from an insertion, a deletion, and a substitution in the endogenous PD1 sequence. In some embodiments, at least 70% of cells in the population comprise a modification selected from an insertion, a deletion, and a substitution in the endogenous PD1 sequence. In some embodiments, at least 90% of cells in the population comprise a modification selected from an insertion, a deletion, and a substitution in the endogenous PD1 sequence. In some embodiments, at least 95% of cells in the population comprise a modification selected from an insertion, a deletion, and a substitution in the endogenous PD1 sequence. In some embodiments. PD1 is decreased by at least 50%, 55%, 60%, 65%, preferably at least 70%, 75%, 80%, 85%, 90%, 95%, 96%, 97%, 98%, or 99%, or to below the limit of detection of the assay as compared to a suitable control, e.g., wherein the PD1 gene has not been modified. In some embodiments, expression of PD1 is decreased by at least 50%, or to below the limit of detection of the assay as compared to a suitable control, e.g., wherein the PD1 gene has not been modified. In some embodiments, expression of PD1 is decreased by at least 55%, or to below the limit of detection of the assay as compared to a suitable control, e.g., wherein the PD1 gene has not been modified. In some embodiments, expression of PD1 is decreased by at least 60%, or to below the limit of detection of the assay as compared to a suitable control, e.g., wherein the PD1 gene has not been modified. In some embodiments, expression of PD1 is decreased by at least 65%, or to below the limit of detection of the assay as compared to a suitable control, e.g., wherein the PD1 gene has not been modified. In some embodiments, expression of PD1 is decreased by at least 70%, or to below the limit of detection of the assay as compared to a suitable control, e.g., wherein the PD1 gene has not been modified. In some embodiments, expression of PD1 is decreased by at least 80%, or to below the limit of detection of the assay as compared to a suitable control, e.g., wherein the PD1 gene has not been modified. In some embodiments, expression of PD1 is decreased by at least 90%, or to below the limit of detection of the assay as compared to a suitable control. e.g., wherein the PD1 gene has not been modified. In some embodiments, expression of PD1 is decreased by at least 95%, or to below the limit of detection of the assay as compared to a suitable control, e.g., wherein the PD1 gene has not been modified. Assays for PD1 protein and mRNA expression are known in the art.

In some embodiments, the engineered cells or population of cells comprise a modification, e.g., knockdown, of a TCR gene sequence by gene editing, e.g., as assessed by sequencing, e.g., NGS, wherein at least 50%, 55%, 60%, 65%, preferably at least 70%, 75%, 80%, 85%, 90%, 95%, 96%, 97%, 98%, or 99% of cells comprise an insertion, deletion, or substitution in the endogenous TCR gene sequence. In some embodiments, TCR is decreased by at least 50%, 55%, 60%, 65%, preferably at least 70%, 75%, 80%, 85%, 90%, 95%, 96%, 97%, 98%, 99%, or to below the limit of detection of the assay as compared to a suitable control, e.g., wherein the TCR gene has not been modified. In certain embodiments, the TCR is TRAC or TRBC. Assays for TCR protein and mRNA expression are known in the art.

In some embodiments, the engineered cells or population of cells comprise an insertion of sequence(s) encoding a targeting receptor by gene editing, e.g., as assessed by sequencing, e.g., NGS.

In some embodiments, guide RNAs that specifically target sites within the TCR genes, e.g., TRAC gene, are used to provide a modification, e.g., knockdown, of the TCR genes.

In some embodiments, the TCR gene is modified, e.g., knocked down, in a T cell using a guide RNA with an RNA-guided DNA binding agent. In some embodiments, disclosed herein are T cells engineered by inducing a break (e.g., double-stranded break (DSB) or single-stranded break (nick)) within the TCR genes of a T cell, e.g., using a guide RNA with an RNA-guided DNA-binding agent (e.g., a CRISPR/Cas system). The methods may be used in vitro or ex vivo, e.g., in the manufacture of cell products for suppressing immune response.

In some embodiments, the guide RNAs mediate a target-specific cutting by an RNA-guided DNA-binding agent (e.g., Cas nuclease) at a site described herein within a TCR gene. It will be appreciated that, in some embodiments, the guide RNAs comprise guide sequences that bind to, or are capable of binding to, said regions.

### III. Methods and Uses Including Therapeutic Methods and Uses and Methods of Preparing Engineered Cells or Immunotherapy Reagents

The gRNAs and associated methods and compositions disclosed herein are useful for making immunotherapy reagents, such as engineered cells.

In some embodiments, the gRNAs comprising the guide sequences of Table 1 together with an RNA-guided DNA nuclease such as a Cas nuclease induce DSBs, and non-homologous ending joining (NHEJ) during repair leads to a modification, e.g., a mutation, in a PD1 gene. In some embodiments, NHEJ leads to a deletion or insertion of a nucleotide(s), which induces a frame shift or nonsense mutation in a PD1 gene. In certain embodiments, gRNAs comprising guide sequences targeted to TCR sequences, e.g., TRAC and TRBC, are also delivered to the cell together with RNA-guided DNA nuclease such as a Cas nuclease, either together or separately, to make a genetic modification in a TCR sequence to inhibit the expression of a full-length TCR sequence. In certain embodiments, the gRNAs are sgRNAs. In some embodiments, the subject is mammalian. In some embodiments, the subject is human. In some embodiments, the subject is a non-human primate

In some embodiments, the guide RNAs, compositions, and formulations are used to produce a cell ex vivo, e.g., an immune cell, e.g., a T cell with a genetic modification in a PD1 gene. The modified T cell may be a natural killer (NK) T-cell. The modified T cell may express a T-cell receptor, such as a universal TCR or a modified TCR. The T cell may express a CAR or a CAR construct with a zeta chain signaling motif.

Delivery of gRNA Compositions

Lipid nanoparticles (LNPs) are a well-known means for delivery of nucleotide and protein cargo, and may be used for delivery of the guide RNAs and compositions disclosed herein ex vivo and in vitro. In some embodiments, the LNPs deliver nucleic acid, protein, or nucleic acid together with protein.

In some embodiments, a method for delivering any one of the cells or populations of cells disclosed herein to a subject is provided, wherein the gRNA is delivered via an LNP. In some embodiments, the gRNA/LNP is also associated with a Cas9 or an mRNA encoding Cas9.

In some embodiments, a composition comprising any one of the gRNAs disclosed and an LNP is provided. In some embodiments, the composition further comprises a Cas9 or an mRNA encoding Cas9.

In some embodiments, LNPs associated with the gRNAs disclosed herein are for use in preparing cells as a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, a method for delivering any one of the gRNAs disclosed herein to an ex vivo cell is provided, wherein the gRNA is associated with an LNP or not associated with an LNP. In some embodiments, the gRNA/LNP or gRNA is also associated with a Cas9 or an mRNA encoding Cas9.

In some embodiments, the guide RNA compositions described herein, alone or encoded on one or more vectors, are formulated in or administered via a lipid nanoparticle; see e.g., WO2017/173054 and WO2021/222287, the contents of each of which are hereby incorporated by reference in their entirety.

In certain embodiments, DNA or RNA vectors encoding any of the guide RNAs comprising any one or more of the guide sequences described herein are provided. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### IV. EXAMPLES

The following examples are provided to illustrate certain disclosed embodiments and are not to be construed as limiting the scope of this disclosure in any way.

### Example 1. Materials and Methods

### 1.1. Next-Generation Sequencing (“NGS”) and Analysis for On-Target Cleavage Efficiency

Genomic DNA was extracted using QuickExtract™ DNA Extraction Solution (Lucigen, Cat. No. QE09050) according to manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., PD1), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “indel percent”) is defined as the total number of sequence reads with insertions or deletions (“indels”) over the total number of sequence reads, including wild type.

### 1.2. Preparation of Lipid Nanoparticles

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z, 12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 by weight, unless otherwise specified.

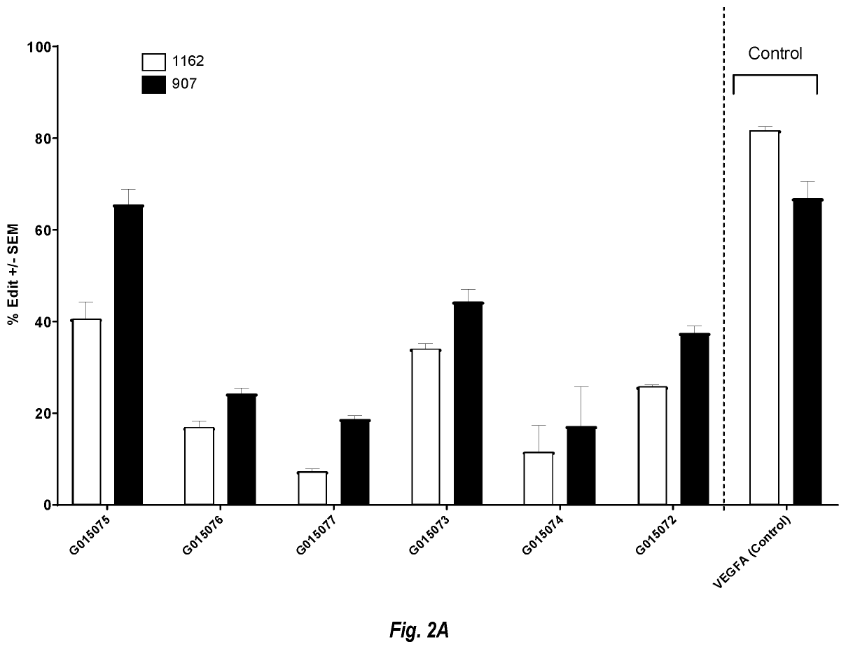

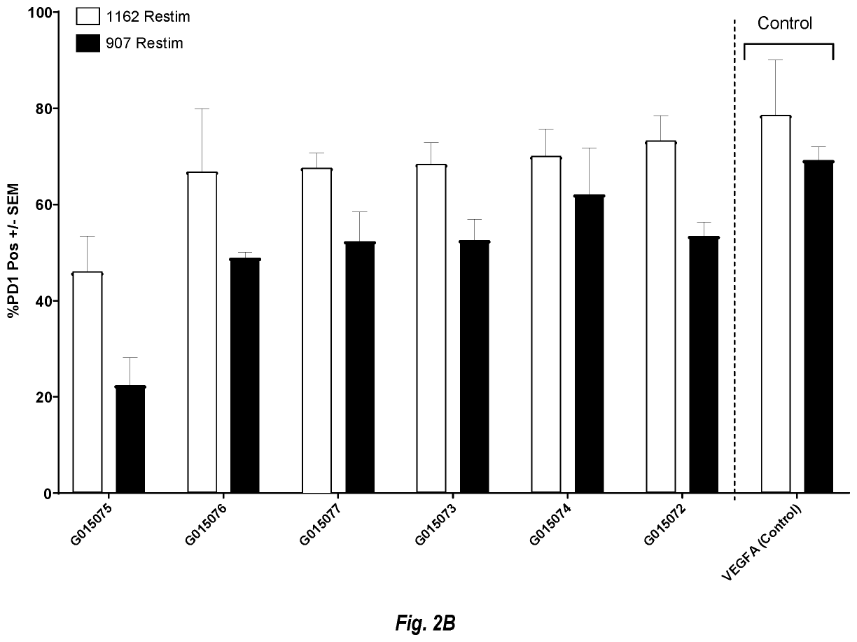

Lipid nanoparticles (LNPs) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2). The LNPs were held for 1 hour at room temperature (RT), and further diluted with water (approximately 1:1 v/v). LNPs were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° ° C. until further use.

1.3. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37ºC for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37ºC for 1.5-4 hours in the following conditions: 50 ng/μL linearized plasmid: 2-5 mM each of GTP, ATP, CTP, and N1-methyl pseudo-UTP (Trilink); 10-25 mM ARCA (Trilink): 5 U/μL T7 RNA polymerase (NEB): 1 U/μL Murine RNase inhibitor (NEB): 0.004 U/μL Inorganic E, coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID NOs: 801-803 (see sequences in Table 12). When SEQ ID NOs: 801-803 are referred to below with respect to RNAs, it is understood that Ts should be replaced with Us (which were N1-methyl pseudouridines as described above). Messenger RNAs used in the Examples include a 5′ cap and a 3′ poly-A tail, e.g., up to 100 nts, and are identified by the SEQ ID NOs: 801-803 in Table 12. Guide RNAs are chemically synthesized by methods known in the art.

### Example 2—PD1 Guide Design and Screening in HEK Cells

### 2.1 Genomic DNA Isolation

HEK293_Cas9 transfected cells were harvested post-transfection at 48 hours. The gDNA was extracted from each well of a 96-well plate using 50 μL/well QuickExtract™ DNA Extraction Solution (Lucigen, Cat. QE09050) according to manufacturer's protocol. DNA samples were subjected to PCR and subsequent NGS analysis, as described herein.

### 2.2 Human PD1 Guide Design

Initial guide selection was performed in silico using a human reference genome (e.g., hg38) and user defined genomic regions of interest (e.g., PD1 protein coding exons), for identifying PAMs in the regions of interest. For each identified PAM, analyses were performed and statistics reported, gRNA molecules were further selected and rank-ordered based on a number of criteria known in the art (e.g., GC content, predicted on-target activity, and potential off-target activity).

A total of 88 guide RNAs were designed toward PD1 (ENST00000334409). Guide sequences and corresponding genomic coordinates are provided in Table 1. For each crRNA, the guide sequence indicated by SEQ ID NO is included within an N20GUUUUAGAGCUAUGCUGUUUUG (SEQ ID NO: 203) nucleic acid sequence, where “N20” represents the guide sequence.

Guides were screened for editing efficiency in HEK293_Cas9 cells. The human embryonic kidney adenocarcinoma cell line HEK293 constitutively expressing Spy Cas9 (“HEK293_Cas9”) was cultured in DMEM media supplemented with 10% fetal bovine serum. Cells were plated at a density of 10,000 cells/well in a 96-well plate about 24 hours prior to transfection (˜70% confluent at time of transfection). Cells were transfected with Lipofectamine RNAiMAX (ThermoFisher, Cat. 13778150) according to the manufacturer's protocol. Cells were transfected with a lipoplex containing crRNA (25 nM), trRNA (25 nM), Lipofectamine RNAiMAX (0.3 μL/well) and OptiMem Medium (ThermoFisher). DNA isolation and NGS analysis were performed as described in Example 1. Table 4 shows % editing at the PD1 locus by these guides in HEK293_Cas9 cells.

TABLE 4
PD1 guide editing in HEK293_Cas9 cells

| | % Editing | % Editing | Number of |
|---|---|---|---|
| crRNA | (Mean) | (St. Dev) | samples (n) |
| CR000833 | NA | NA | 3 |
| CR000834 | NA | NA | 3 |
| CR000836 | NA | NA | 3 |
| CR000837 | 49.37 | 9.77 | 3 |
| CR000838 | 76.40 | 5.11 | 3 |
| CR000839 | 95.23 | 0.55 | 2 |
| CR000840 | 49.37 | 4.88 | 3 |
| CR000842 | 81.20 | 3.10 | 3 |
| CR000844 | 43.63 | 9.71 | 3 |
| CR000845 | 33.17 | 4.43 | 3 |
| CR000846 | 71.57 | 10.45 | 3 |
| CR000847 | 89.97 | 1.96 | 3 |
| CR000849 | 46.30 | 8.94 | 3 |
| CR000850 | 65.27 | 8.47 | 3 |
| CR000853 | 40.60 | 13.00 | 3 |
| CR000854 | 56.20 | 7.53 | 3 |
| CR000856 | 81.67 | 1.72 | 3 |
| CR000857 | 17.23 | 3.26 | 3 |
| CR000858 | 41.73 | 10.08 | 3 |
| CR000860 | 4.50 | 0.87 | 3 |
| CR000868 | 75.47 | 3.90 | 3 |
| CR000869 | 87.60 | 3.03 | 3 |
| CR000870 | 91.43 | 1.36 | 3 |
| CR000871 | 79.13 | 2.42 | 3 |
| CR000872 | 26.33 | 4.27 | 3 |
| CR000874 | 63.63 | 6.37 | 3 |
| CR000875 | 24.63 | 3.80 | 3 |
| CR000876 | 38.37 | 2.61 | 3 |
| CR000879 | 93.97 | 0.40 | 3 |
| CR000880 | 17.23 | 2.98 | 3 |
| CR000885 | 8.50 | 2.33 | 3 |
| CR000889 | 1.27 | 0.15 | 3 |
| CR000890 | 3.27 | 1.60 | 3 |
| CR000894 | 16.90 | 0.36 | 3 |
| CR000901 | 51.60 | 11.72 | 3 |
| CR000902 | 94.47 | 1.91 | 3 |
| CR000903 | 82.73 | 3.33 | 3 |
| CR000904 | 86.57 | 2.89 | 3 |
| CR000908 | NA | NA | 3 |
| CR000911 | 58.73 | 3.09 | 3 |
| CR000912 | 81.33 | 7.07 | 3 |
| CR000913 | 69.50 | 8.23 | 3 |
| CR002588 | 84.60 | 1.81 | 3 |
| CR007233 | 0.27 | 0.06 | 3 |
| CR007234 | 0.13 | 0.06 | 3 |
| CR007235 | 0.17 | 0.06 | 3 |
| CR007236 | 0.27 | 0.15 | 3 |
| CR007237 | NA | NA | 3 |
| CR007238 | 0.03 | 0.06 | 3 |
| CR007239 | 2.43 | 0.57 | 3 |
| CR007240 | 21.83 | 13.67 | 3 |
| CR007241 | 2.47 | 0.93 | 3 |
| CR007242 | 21.73 | 3.78 | 3 |
| CR007243 | 0.27 | 0.06 | 3 |
| CR007244 | 0.13 | 0.06 | 3 |
| CR007245 | 86.03 | 2.19 | 3 |
| CR007246 | NA | NA | 3 |
| CR007247 | 85.03 | 2.08 | 3 |
| CR007248 | 73.70 | 9.15 | 3 |
| CR007249 | 0.25 | 0.35 | 3 |
| CR007250 | 2.33 | 0.35 | 3 |
| CR007251 | 0.50 | 0.10 | 3 |
| CR007252 | 21.30 | 7.52 | 3 |
| CR007253 | 10.83 | 4.04 | 3 |
| CR007254 | 1.10 | 0.26 | 3 |
| CR007255 | 1.00 | 0.26 | 3 |
| CR007256 | NA | NA | 3 |
| CR007257 | 0.23 | 0.12 | 3 |
| CR007258 | 1.27 | 0.59 | 3 |
| CR007259 | 3.40 | 1.11 | 3 |
| CR007260 | 3.50 | 1.00 | 3 |
| CR007261 | 0.90 | 0.35 | 3 |
| CR007262 | 0.20 | 0.00 | 3 |
| CR007263 | 5.10 | 2.40 | 3 |
| CR007264 | 0.27 | 0.15 | 3 |
| CR007265 | 0.93 | 0.47 | 3 |
| CR007266 | NA | NA | 3 |
| CR007267 | 0.27 | 0.06 | 3 |
| CR007268 | 56.30 | 8.43 | 3 |
| CR007269 | 82.17 | 5.45 | 3 |
| CR007270 | 8.87 | 3.72 | 3 |
| CR007271 | 80.67 | 4.40 | 3 |
| CR007272 | NA | NA | 3 |
| CR007273 | 0.40 | 0.17 | 3 |
| CR007274 | 52.77 | 5.25 | 3 |
| CR007275 | 17.30 | 2.33 | 3 |
| CR007276 | 0.00 | NA | 3 |
| CR007277 | 79.63 | 5.05 | 3 |
| CR003187 VEGFA | 70.87 | 3.15 | 3 |

### Example 3—PD1 Guide Screening in Human CD3⁺ T Cells

Guides from the editing screen in HEK293_Cas9 cells from Example 2 were screened for editing efficiency in human CD3⁺ T cells. CD3⁺ T cells are comprised of multiple T cell populations including CD4⁺ T helper cells and CD8⁺ cytotoxic T cells. These cells can be isolated from whole blood or from leukopheresis samples. T cells can be modified to specifically target cancerous cells and to be less immunogenic, by engineering T cells using Cas9-mediated editing.

### Delivery of RNPs to T Cells

T cells were either obtained commercially (e.g. Human Peripheral Blood T Cells, Frozen, Stem Cell Technology, Cat. 70029) or prepared internally from a leukopak. For internal preparation, T cells were isolated using a commercially available kit (e.g., EasySep™ Human T Cell Isolation Kit, Stem Cell Technology). T cells were aliquoted and frozen down (at 5×10⁶/vial) for future use. Vials were subsequently thawed as needed, and activated by addition of 3:1 ratio of Dynabeads Human T-Expander CD3/CD28 (Life Technologies 11141D) in T cell media (RPMI 1640, FBS, L-glutamine, non-essential amino acids, sodium pyruvate, HEPES buffer, 2-mercaptoethanol and IL2). RNP was generated by pre-annealing individual crRNA and trRNA by mixing equivalent amounts of reagent and incubating at 95° C. for 2 min and cooling to room temperature. The dual guide (dgRNA) consisting of pre-annealed crRNA and trRNA, was incubated with Spy Cas9 protein to form a ribonucleoprotein (RNP) complex. CD3⁺ T cells were transfected in technical triplicates with an RNP containing Spy Cas9 (10 nM), crRNA (10 nM) and tracer RNA (10 nM) nucleofected using the P3 Primary Cell 96-well Nucleofector™ Kit (Lonza, Cat. V4SP-3960) using the manufacturer's Amaxa™ 96-well Shuttle™ Protocol for Stimulated Human T Cells. T cell media was added to cells immediately post-nucleofection and cultured for 2 days or more.

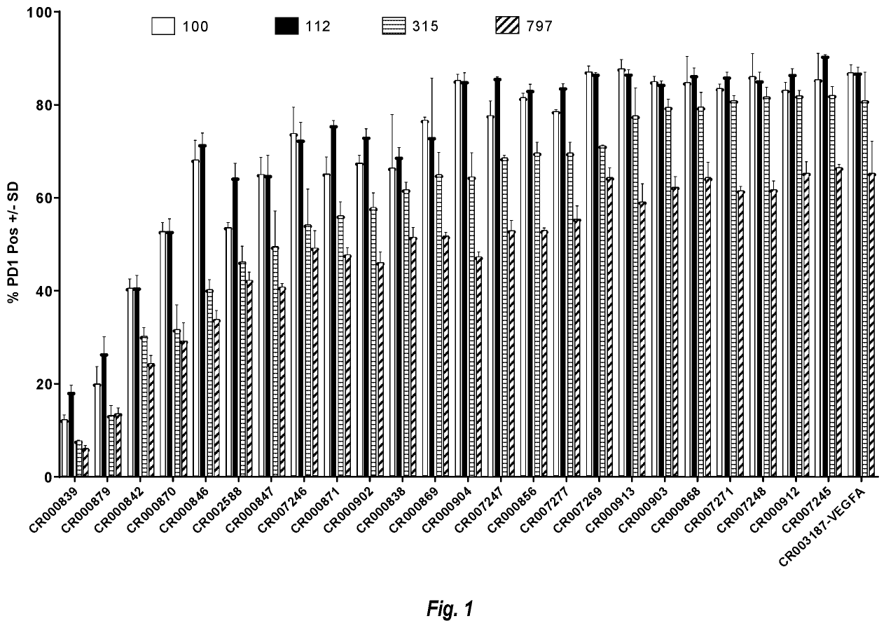

Expression of the PD1 was measured 4 days post nucleofection. Cells were stained with fixable live dead dye (Thermo fisher L34975) and the PD1 was detected using of Pe-cy 7 anti-human Antibody (Biolegend, Cat. 329918). Cells were incubated with 1 ul of antibody for at least 20 minutes one ice, analyzed by flow cytometry using, for example, Beckman Coulter CytoflexS. Data was analyzed using Flow Jo software. Loss of PD1 protein expression was determined by gating on live cells and PD1 expression. The results of flow cytometry analysis at 4 days post nucleofection are shown in Table 5 and FIG. 1.

TABLE 5
Percentage of PD-1 positive human CD3+ T cells 4 days after dual guide editing
Table 5 - Percentage of PD-1 positive human CD3⁺ T cells after dual guide editing

| | Donor 315 | | Donor 797 | | Donor 100 | | Donor 112 | |
|---|---|---|---|---|---|---|---|---|
| | Mean % + | SD | Mean % + | SD | Mean % + | SD | Mean % + | SD |
| crRNA | PD-1 | (%) | PD-1 | (%) | PD-1 | (%) | PD-1 | (%) |
| CR000828 | 81.0 | 2.9 | 64.4 | 3.7 | 89.7 | 1.4 | 90.0 | 1.0 |
| CR000838 | 61.8 | 1.3 | 51.6 | 1.7 | 66.5 | 9.4 | 68.8 | 1.6 |
| CR000839 | 7.7 | 0.1 | 6.0 | 0.6 | 12.3 | 0.8 | 18.2 | 1.2 |
| CR000842 | 30.2 | 1.5 | 24.3 | 1.4 | 40.6 | 1.6 | 40.7 | 2.2 |
| CR000846 | 40.3 | 1.8 | 33.8 | 1.6 | 68.2 | 3.4 | 71.5 | 2.0 |
| CR000847 | 49.5 | 6.3 | 40.8 | 0.6 | 65.1 | 3.0 | 64.9 | 3.5 |
| CR000856 | 69.7 | 1.9 | 52.9 | 0.5 | 81.5 | 0.9 | 83.2 | 1.0 |
| CR000868 | 79.5 | 2.6 | 64.3 | 2.8 | 84.8 | 4.6 | 86.4 | 1.2 |
| CR000869 | 64.9 | 3.9 | 51.7 | 0.7 | 76.7 | 0.5 | 73.1 | 10.4 |
| CR000870 | 31.7 | 4.3 | 29.2 | 3.2 | 52.8 | 1.5 | 52.8 | 2.2 |
| CR000871 | 56.2 | 2.4 | 47.7 | 1.2 | 65.2 | 2.9 | 75.6 | 0.8 |
| CR000879 | 13.2 | 1.7 | 13.6 | 1.0 | 20.0 | 3.0 | 26.5 | 2.9 |
| CR000902 | 57.8 | 2.6 | 46.1 | 1.8 | 67.6 | 1.3 | 73.2 | 1.4 |
| CR000903 | 79.5 | 1.4 | 62.3 | 1.8 | 85.0 | 0.9 | 84.5 | 0.5 |
| CR000904 | 64.5 | 4.2 | 47.3 | 0.9 | 85.2 | 1.1 | 85.1 | 1.5 |
| CR000912 | 82.0 | 0.9 | 65.3 | 2.0 | 83.2 | 1.3 | 86.6 | 0.9 |
| CR000913 | 77.6 | 4.9 | 59.2 | 3.2 | 87.8 | 1.6 | 86.7 | 0.7 |
| CR002588 | 46.2 | 2.7 | 42.3 | 1.4 | 53.7 | 0.8 | 64.3 | 2.5 |
| CR007245 | 82.1 | 1.6 | 66.5 | 0.6 | 85.5 | 4.6 | 90.5 | 0.2 |
| CR007246 | 54.2 | 6.3 | 49.2 | 3.0 | 73.8 | 4.6 | 72.5 | 3.1 |
| CR007247 | 68.5 | 0.5 | 53.0 | 1.7 | 77.7 | 2.6 | 85.8 | 0.2 |
| CR007248 | 81.8 | 1.6 | 61.8 | 1.5 | 86.1 | 4.0 | 85.2 | 1.4 |
| CR007269 | 71.2 | 0.1 | 64.4 | 1.7 | 87.2 | 1.0 | 86.7 | 0.2 |
| CR007271 | 80.9 | 0.8 | 61.6 | 0.7 | 83.5 | 0.8 | 86.1 | 0.8 |
| CR007277 | 69.6 | 1.9 | 55.5 | 2.2 | 78.6 | 0.3 | 83.8 | 0.6 |
| CR003187 VEGFA | 80.9 | 5.0 | 65.3 | 5.6 | 86.9 | 1.4 | 86.9 | 0.9 |

To confirm that PD-I protein loss was due to gene editing. NGS analysis was performed. Two days post nucleofection, genomic DNA was prepared from treated cells and NGS analysis performed, as described in Example 1. Tables 6A and 6B how results for indel frequency following PD1 editing with various guides in CD3⁺ T cells.

TABLE 6A
Mean percent editing using dual guides in human CD3+ T cells

| | Donor 315 Primer Set 1 | | | Donor 315 Primer Set 2 | | | Donor 797 Primer Set 1 | | | Donor 797 Primer Set 2 | | |
|---|---|---|---|---|---|---|---|---|---|---|---|---|
| | Mean | SD | | Mean | SD | | Mean | SD | | Mean | SD | |
| crRNA | % Edit | (%) | n | % Edit | (%) | n | % Edit | (%) | n | % Edit | (%) | n |
| CR000838 | 25.9 | 1.9 | 3 | 20.7 | 1.0 | 3 | 23.7 | 0.5 | 3 | 18.0 | 0.3 | 3 |
| CR000839 | 61.9 | 3.9 | 3 | 57.9 | 6.4 | 3 | 49.3 | 1.7 | 3 | 42.1 | 3.2 | 3 |
| CR000842 | 41.0 | 3.1 | 3 | 34.3 | 2.6 | 3 | 30.2 | 1.3 | 3 | 24.9 | 0.2 | 3 |
| CR000846 | 33.2 | 1.6 | 3 | 30.5 | 2.6 | 3 | 21.2 | 3.9 | 3 | 18.5 | 1.8 | 3 |
| CR000847 | 37.2 | 0.9 | 3 | 33.5 | 3.4 | 3 | 23.1 | 1.1 | 3 | 22.5 | 2.8 | 3 |
| CR000856 | 17.8 | 0.7 | 3 | 16.0 | 1.5 | 3 | 9.2 | 0.5 | 3 | 10.2 | 0.5 | 3 |
| CR000868 | 8.5 | 0.6 | 3 | 8.6 | 0.4 | 3 | 5.8 | 0.6 | 3 | 5.3 | 0.6 | 3 |
| CR000869 | 15.5 | 1.0 | 3 | 13.7 | 1.1 | 3 | 11.4 | 0.7 | 3 | 9.6 | 1.6 | 3 |
| CR000870 | 50.1 | 2.7 | 3 | 49.2 | 1.0 | 3 | 40.9 | 3.5 | 3 | 39.6 | 2.8 | 3 |
| CR000871 | 30.6 | 0.8 | 3 | 26.8 | 2.1 | 3 | 21.7 | 0.7 | 3 | 19.9 | 1.8 | 3 |
| CR000879 | 48.0 | 2.8 | 3 | 51.5 | 1.3 | 3 | 55.5 | 3.6 | 3 | 56.6 | 2.0 | 3 |
| CR000902 | 41.2 | 1.0 | 3 | 41.7 | 4.5 | 3 | 30.0 | 2.5 | 3 | 29.4 | 1.3 | 3 |
| CR000903 | 19.0 | 0.6 | 3 | 18.1 | 1.4 | 3 | 12.0 | 0.3 | 3 | 11.6 | 1.1 | 3 |
| CR000904 | 7.0 | 0.3 | 3 | 8.4 | 0.6 | 3 | 5.9 | 0.7 | 3 | 5.9 | 0.3 | 3 |
| CR000912 | 21.2 | 5.9 | 3 | 23.2 | 10.5 | 3 | 15.8 | 2.4 | 3 | 13.9 | 1.3 | 3 |
| CR000913 | 7.6 | 0.9 | 3 | 8.0 | 2.9 | 3 | 7.5 | 5.6 | 3 | 10.8 | 4.4 | 3 |
| CR002588 | 27.5 | 2.2 | 3 | 27.6 | 1.6 | 3 | 17.4 | 1.2 | 3 | 15.4 | 0.6 | 3 |
| CR007245 | 12.3 | 1.1 | 3 | 15.7 | 1.1 | 3 | 7.7 | 0.4 | 3 | 8.4 | 0.7 | 3 |
| CR007246 | 45.2 | 4.5 | 3 | No data | N/A | 0 | 22.6 | 2.8 | 3 | No data | N/A | 0 |
| CR007247 | 16.5 | 0.6 | 3 | 14.5 | 0.8 | 3 | 9.6 | 1.0 | 3 | 9.8 | 0.9 | 3 |
| CR007248 | 17.0 | 1.4 | 3 | 17.6 | 1.2 | 3 | 9.6 | 0.2 | 3 | 11.0 | 1.9 | 3 |
| CR007269 | 14.2 | 1.2 | 3 | 13.5 | 0.8 | 3 | 7.4 | 1.2 | 3 | 7.9 | 1.3 | 3 |
| CR007271 | 28.0 | 2.2 | 3 | 22.8 | 1.7 | 3 | 12.6 | 0.8 | 3 | 11.3 | 0.7 | 3 |
| CR007277 | 20.0 | 0.9 | 3 | 18.8 | 0.7 | 3 | 12.8 | 1.1 | 3 | 12.2 | 0.4 | 3 |
| CR003187 | 12.5 | 0.6 | 3 | 12.7 | 2.0 | 3 | 6.0 | 0.7 | 3 | 6.7 | 0.4 | 3 |

TABLE 6B
Mean percent editing in human CD3+ T cells

| | Donor 100 Primer Set 1 | | | Donor 100 Primer Set 2 | | | Donor 112 Primer Set 1 | | | Donor 112 Primer Set 2 | | |
|---|---|---|---|---|---|---|---|---|---|---|---|---|
| | Mean | SD | | Mean | SD | | Mean | SD | | Mean | SD | |
| Guide | % Edit | (%) | n | % Edit | (%) | n | % Edit | (%) | n | % Edit | (%) | n |
| CR000838 | 37.9 | 5.4 | 3 | 30.1 | 5.7 | 3 | 21.6 | 1.0 | 3 | 13.8 | 2.7 | 3 |
| CR000839 | 81.9 | 1.5 | 3 | 77.3 | 4.9 | 3 | 43.7 | 4.5 | 3 | 37.7 | 5.7 | 3 |
| CR000842 | 65.0 | 2.3 | 3 | 55.6 | 1.7 | 3 | 33.0 | 2.3 | 3 | 27.1 | 5.1 | 3 |
| CR000846 | 33.8 | 1.9 | 3 | 35.3 | 2.5 | 3 | 14.1 | 2.1 | 3 | 13.9 | 0.7 | 3 |
| CR000847 | 47.3 | 2.1 | 3 | 46.9 | 1.4 | 3 | 22.9 | 4.2 | 3 | 24.3 | 3.0 | 3 |
| CR000856 | 20.4 | 0.4 | 3 | 20.0 | 0.9 | 3 | 8.3 | 1.7 | 3 | 7.9 | 0.8 | 3 |
| CR000868 | 9.7 | 2.2 | 3 | 9.0 | 2.2 | 3 | 6.0 | 1.1 | 3 | 6.4 | 1.9 | 3 |
| CR000869 | 20.3 | 1.2 | 3 | 20.6 | 2.3 | 3 | 10.7 | 1.4 | 3 | 17.2 | 14.2 | 3 |
| CR000870 | 51.9 | 2.5 | 3 | 48.9 | 1.4 | 3 | 35.9 | 2.6 | 3 | 35.2 | 2.6 | 3 |
| CR000871 | 37.5 | 0.7 | 3 | 35.3 | 1.8 | 3 | 18.8 | 1.2 | 3 | 14.7 | 0.7 | 3 |
| CR000879 | 67.7 | 6.2 | 3 | 67.4 | 5.9 | 3 | No data | N/A | 0 | 38.3 | 3.5 | 3 |
| CR000902 | 55.3 | 1.8 | 3 | 53.0 | 1.8 | 3 | 29.8 | 1.4 | 3 | 31.7 | 2.8 | 3 |
| CR000903 | 26.1 | 3.0 | 3 | 25.2 | 1.3 | 3 | 11.8 | 1.7 | 3 | 11.6 | 2.2 | 3 |
| CR000904 | 10.9 | 0.5 | 3 | 10.8 | 0.5 | 3 | 6.0 | 0.9 | 3 | 6.0 | 0.5 | 3 |
| CR000912 | 100.0 | N/A | 1 | 99.6 | N/A | 1 | No data | N/A | 0 | No data | N/A | 0 |
| CR000913 | 38.3 | 14.5 | 3 | 41.2 | 25.7 | 2 | 4.6 | 1.6 | 3 | No data | N/A | 0 |
| CR002588 | 45.1 | 5.0 | 3 | 39.9 | 2.2 | 3 | 15.0 | 3.2 | 3 | 11.2 | 2.6 | 3 |
| CR007245 | 12.6 | 5.1 | 3 | 16.8 | 5.7 | 3 | 7.3 | 2.0 | 3 | 8.0 | 1.1 | 3 |
| CR007246 | 99.4 | 0.2 | 3 | No data | N/A | 0 | 99.0 | N/A | 1 | No data | N/A | 0 |
| CR007247 | 19.0 | 1.9 | 3 | 19.5 | 0.9 | 3 | 8.2 | 0.6 | 3 | 6.1 | 1.8 | 3 |
| CR007248 | 24.2 | 6.9 | 3 | 29.1 | 9.3 | 3 | 11.5 | 1.6 | 3 | 12.6 | 1.5 | 3 |
| CR007269 | 17.8 | 0.9 | 3 | 17.9 | 1.3 | 3 | 6.6 | 1.1 | 3 | 5.7 | 0.2 | 3 |
| CR007271 | 37.4 | 2.4 | 3 | 35.7 | 1.1 | 3 | 14.5 | 4.2 | 3 | 15.0 | 4.9 | 3 |
| CR007277 | 36.9 | 6.7 | 3 | 34.7 | 1.4 | 3 | 20.2 | 3.8 | 3 | 19.3 | 2.0 | 3 |
| CR003187 | 86.5 | 3.1 | 3 | 92.4 | 2.4 | 3 | 66.0 | 16.0 | 3 | 77.0 | 12.3 | 3 |
| VEGFA | | | | | | | | | | | | |

### Example 4—Off-Target Analysis of PD1 Guides

A biochemical method (See, e.g., Cameron et al., Nature Methods. 6, 600-606; 2017) was used to determine potential off-target genomic sites cleaved by Cas9 using guides targeting PD1. Select guides in Example 3 were tested for potential off-target genomic cleavage sites with this assay. In this experiment, 16 dgRNA targeting human PD1 were screened in triplicate using genomic DNA purified from HEK293 cells alongside a positive control guide, VEGFA (G000645) with known off-target profiles. The number of potential off-target sites detected using a guide concentration of 192 nM and 64 nM Cas9 protein in the biochemical assay are shown in Table 7.

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically over represents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

In one approach, primary T cells are treated with LNPs comprising Cas9 mRNA and a sgRNA of interest (e.g., a sgRNA having potential off-target sites for evaluation). The primary T cells are then lysed and primers flanking the potential off-target site(s) are used to generate an amplicon for NGS analysis. Identification of indels at a certain level may validate potential off-target site, whereas the lack of indels found at the potential off-target site may indicate a false positive in the off-target assay that was utilized.

TABLE 7
Potential Off-target sites for PD1guides
predicted by biochemical assay

| Guide | | | | |
|---|---|---|---|---|
| Sequence | | | | |
| SEQ ID | Guide | Target | Site Count | Site Score |
| 8 | CR000842 | PD1 | 47 | 362 |
| 12 | CR000847 | PD1 | 29 | 566 |
| 5 | CR000838 | PD1 | 35 | 1212 |
| 43 | CR002588 | PD1 | 31 | 1392 |
| 22 | CR000869 | PD1 | 58 | 1410 |
| 58 | CR007247 | PD1 | 41 | 1672 |
| 17 | CR000856 | PD1 | 48 | 2446 |
| 24 | CR000871 | PD1 | 79 | 3064 |
| 38 | CR000904 | PD1 | 128 | 3330 |
| 23 | CR000870 | PD1 | 83 | 3388 |
| 11 | CR000846 | PD1 | 104 | 4207 |
| 57 | CR007246 | PD1 | 114 | 5921 |
| 6 | CR000839 | PD1 | 140 | 6398 |
| 36 | CR000902 | PD1 | 148 | 8262 |
| 29 | CR000879 | PD1 | 125 | 8492 |
| 56 | CR007245 | PD1 | 668 | 88859 |
| 108 | G000645 | VEGFA | 1031 | 223376 |
| | | Control | | |

### Example 5—Single Guide Analysis in CD3+ T Cells

### 5.1 Delivery of RNPs to T Cells

T cells were prepared as outlined in Example 3. Single guide (sgRNA) was incubated at 95° C. for 2 min and cooling to room temperature. Then the sgRNA was incubated with Spy Cas9 protein to form a ribonucleoprotein (RNP) complex. CD3⁺ T cells were transfected with an RNP containing Spy Cas9 (10 nM) and individual sgRNA (10 nM) nucleofected using the P3 Primary Cell 96-well Nucleofector™ Kit (Lonza, Cat. V4SP-3960) using the manufacturer's Amaxa™ 96-well Shuttle™ Protocol for Stimulated Human T Cells. T cell media was added to cells immediately post-nucleofection and cultured for 10 more days before harvesting and performing NGS as in Example 1.

TABLE 8A
Restimulated PD1 NGS Editing

| | |
|---|---|
| Donor 1162 | Donor 907 |

| | Mean % | SD % | Mean % | SD % |
|---|---|---|---|---|
| Guide | Editing | Editing | Editing | Editing |
| G015075 | 40.53 | 6.46 | 65.57 | 5.78 |
| G015076 | 16.87 | 2.49 | 24.27 | 2.15 |
| G015077 | 7.30 | 1.01 | 18.73 | 1.25 |
| G015073 | 34.00 | 2.12 | 44.33 | 4.70 |
| G015074 | 11.57 | 9.97 | 17.20 | 14.91 |
| G015072 | 25.70 | 0.96 | 37.50 | 2.75 |
| VEGFA (Control) | 81.70 | 1.42 | 66.85 | 6.45 |

On day seven post electroporation, media was prepared with IL-2 and CD3/CD28 beads (Dynabeads). The cell to bead ratio was 1:1 for restimulation. Restimulated editing levels were measured by NGS as in Example 1 and shown in FIG. 2A and Table 8A. Restimulated protein levels were measured by flow cytometry as in Example 3.2 and shown in FIG. 2B and Table 8B.

TABLE 8B
PD1 Restimulated Levels

| | |
|---|---|
| Donor 1162 | Donor 907 |

| | Mean % | SD % | Mean % | SD % |
|---|---|---|---|---|
| | PD1 +/− | PD1 +/− | PD13 +/− | PD1 +/− |
| Guide | SEM | SEM | SEM | SEM |
| G015075 | 45.87 | 7.60 | 22.43 | 5.78 |
| G015076 | 66.80 | 13.17 | 48.90 | 1.20 |
| G015077 | 67.57 | 3.16 | 52.37 | 6.11 |
| G015073 | 68.33 | 4.53 | 52.57 | 4.39 |
| G015074 | 69.97 | 5.75 | 62.13 | 9.66 |
| G015072 | 73.13 | 5.34 | 53.43 | 2.94 |
| G000645 | 78.53 | 11.52 | 69.23 | 2.80 |
| VEGFA | | | | |
| (Control) | | | | |

### Example 6—PD1 Editing with Various Doses of RNA

T cells were edited with increasing amounts of lipid nanoparticles co-formulated with mRNA encoding Cas9 and a sgRNA targeting PD1 or control loci. Cryopreserved T-cells were thawed in a water bath. T-cells were resuspended at a density of 15×10⁶ per 10 mL of cytokine media. TransAct™ (Miltenyi) was added at a 1:100 dilution to each flask and was incubated at 37° C. overnight.

### 6.1.—LNP Incubation

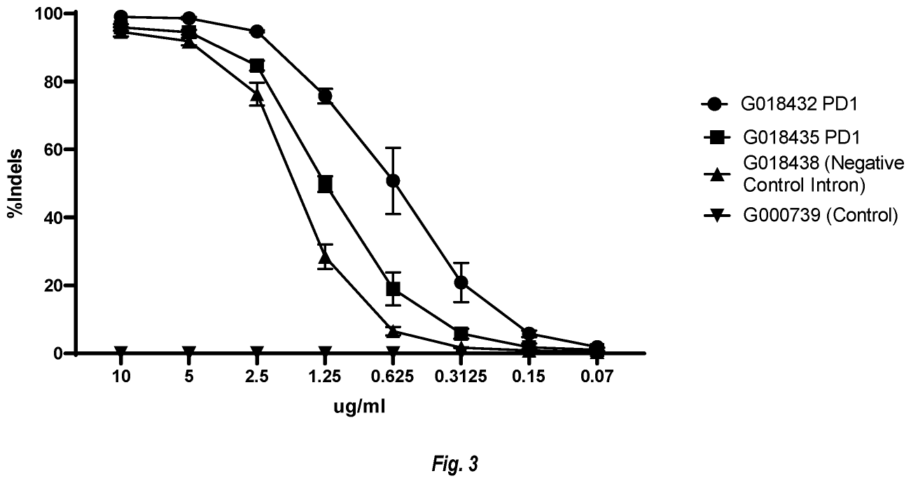

T-cells were harvested and resuspended in Media (XVIVO base media without serum) prepared with cytokines (IL-2 (200 U/mL), IL-7 (5 ng/ml), and IL-15 (5 ng/ml)). ApoE3 was added to a final concentration of 1 ug/mL in XVIVO 5% HS media. LNPs were prepared to a 2× final concentration in the ApoE media and were incubated at 37° C. for 15 minutes. 50 μL of the LNP-ApoE and 50 μL of T-cells were mixed and incubated for 24 hours. NGS analysis was performed as in Example 1. NGS data is shown in Table 9 and a dose response curve is shown in FIG. 3.

TABLE 9
Percent indels for T cells editing with various doses of LNP

| | | | | | G018438 | | | |
|---|---|---|---|---|---|---|---|---|
| | | | | | (Negative Control | | | |
| | G018432 PD1 | | G018435 PD1 | | Intron Guide) | | G000739(Control) | |
| Dose | Average | SD | Average | SD | Average | SD | Average | SD |
| (μg/ml) | % indels | % indels | % indels | % indels | % indels | % indels | % indels | % indels |
| 10 | 99.07 | 0.21 | 95.93 | 0.99 | 94.63 | 1.36 | 0.17 | 0.06 |
| 5 | 98.63 | 0.38 | 94.50 | 0.72 | 91.80 | 1.11 | 0.10 | 0.00 |
| 2.5 | 94.77 | 0.32 | 84.70 | 1.47 | 76.30 | 3.38 | 0.17 | 0.06 |
| 1.25 | 75.73 | 2.15 | 49.83 | 2.32 | 28.47 | 3.58 | 0.13 | 0.06 |
| 0.625 | 50.80 | 9.73 | 19.00 | 4.86 | 6.53 | 1.23 | 0.10 | 0.00 |
| 0.3125 | 20.87 | 5.76 | 5.80 | 1.47 | 1.70 | 0.10 | 0.10 | 0.00 |
| 0.15 | 5.80 | 0.99 | 1.80 | 1.14 | 0.87 | 0.29 | 0.10 | 0.00 |
| 0.07 | 1.90 | 0.00 | 1.03 | 0.60 | 0.47 | 0.21 | 0.17 | 0.06 |

### Example 7—Engineered T Cells with Inhibitor Gene Knockouts

T cells were engineered with a series of gene disruptions and insertions. Healthy donor cells were treated sequentially with three LNPs, each LNP co-formulated with mRNA encoding Cas9 and a sgRNA targeting. Cells were first edited to knockout TRBC and TRAC. A transgenic T cell receptor targeting Wilm's tumor antigen (WT1 TCR) (SEQ ID NO: 1001) was then integrated into the TRAC cut site by delivering a homology directed repair template using AAV. Lastly, T cells were edited to knock out PD1 gene.

### 7.1. T Cell Preparation

Healthy human donor apheresis was obtained commercially (Stemcells), washed and re-suspended in PBS with 2% FBS buffer. T cells were isolated via CD3 negative selection kit and CD3 release kit using EasySep Human T cell Isolation Kit (Catalog #17751) and EasySep™ Release Human CD3 Positive Selection Kit (Catalog #17951). T cells were aliquoted into vials and cryopreserved in R10 media with 10% DMSO for future use. The day before initiating T cell editing, cells were thawed and rested overnight in T cell activation media (TCAM): CTS OpTmizer (Thermofisher, Cat. A3705001) supplemented with 2.5 human AB serum (Gemini, Cat. 100-512), 1× GlutaMAX (Thermofisher, Cat.35050061), 10 mM HEPES (Thermofisher, Cat. 15630080), 200 U/mL IL-2 (Peprotech, Cat. 200-02), IL-7 (Peprotech, Cat. 200-07), IL-15 (Peprotech, Cat. 200-15).

### 7.2. LNP Treatment and Expansion of T Cells

On day 1. LNPs containing Cas9 mRNA and sgRNA targeting TRBC (G016239) were incubated at a concentration of 5 μg/mL in TCAM containing 1 μg/mL rhApoE3 (Peprotech, Cat. 350-02), with lipid A, cholesterol, DSPC, and PEG2k-DMG in a 50:39.5:9:1.5 molar ratio, respectively. The lipid nucleic acid assemblies were formulated with a lipid amine to RNA phosphate (N:P) molar ratio of about 6, and a ratio of gRNA to mRNA of 1:2 by weight. Meanwhile, T cells were harvested, washed, and resuspended at a density of 2×10⁶ cells/mL in TCAM with a 1:50 dilution of T Cell TransAct, human reagent (Miltenyi, Cat. 130)-111-160). T cells and LNP-ApoE media were mixed at a 1:1 ratio and T cells plated in culture flasks overnight.

On day 3, T cells were harvested, washed, and resuspended at a density of 1×10⁶ cells/mL in TCAM. LNPs containing Cas9 mRNA and sgRNA targeting TRAC (G013006) were incubated at a concentration of 5 μg/mL in TCAM containing 5 μg/mL rhApoE3 (Peprotech, Cat. 350-02), WT1 TCR-containing at MOI of 3×10⁵ genome copies/cell and Compound 1 at 0.5 μM TRAC LNPs were formulated with lipid A, cholesterol, DSPC, and PEG2k-DMG in a 50:39.5:9:1.5 molar ratio, respectively. The lipid nucleic acid assemblies were formulated with a lipid amine to RNA phosphate (N:P) molar ratio of about 6, and a ratio of gRNA to mRNA of 1:2 by weight. T cells and LNP-ApoE media were mixed at a 1:1 ratio and T cells plated in culture flasks.

On day 4. T cells were harvested, washed, and resuspended at a density of 1×10⁶ cells/mL in TCAM. LNPs containing Cas9 mRNA and the gRNA shown in Table 11. LNPs were incubated at a concentration of 5 μg/mL in TCAM containing 5 μg/mL rhApoE3 (Peprotech, Cat. 350-02). LNP-ApoE solution was then added to the appropriate culture at a 1:1 ratio.

On days 5-11. T cells were transferred to a 24-well GREX plate (Wilson Wolf, Cat. 80192) in T cell expansion media (TCEM): CTS OpTmizer (Thermofisher, Cat. A3705001) supplemented with 5% CTS Immune Cell Serum Replacement (Thermofisher, Cat. A2596101), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), and IL-15 (Peprotech, Cat. 200-15)). Cells were expanded per manufacturers protocols. T-cells were expanded for 6-days, with media exchanges every other day. Cells were counted using a Vi-CELL cell counter (Beckman Coulter) and all samples showed similar fold-expansion.

### 7.3. Quantification of T Cell Editing by Flow Cytometry and NGS

Post expansion, edited T cells were assayed by flow cytometry to determine TCR insertion and memory cell phenotype. T cells were incubated with an antibody cocktail targeting the following molecules: CD4 (Biolegend, Cat. 300538), CD8 (Biolegend, Cat. 301046), Vb8 (Biolegend, Cat. 348104), CD3 (Biolegend, Cat. 317336), CD62L (Biolegend, Cat. 304820), CD45RO (Biolegend, Cat. 304230), CCR7 (Biolegend, Cat. 353214), and CD45RA (Biolegend, Cat. 304134). Cells were subsequently processed on a Cytoflex LX instrument (Beckman Coulter) and data analyzed using the FlowJo software package. The percentage of cells expressing relevant cell surface proteins following sequential T cell engineering are shown in Tables 10A and 10B. Table 10A shows the percentage of CD8+Vb8+ cells with the stem cell memory phenotype (Tscm; CD45RA+ CD62L+). Table 10B shows the percentage of CD8+Vb8+ cells with central memory cell phenotype (Tcm; CD45RO+ CD62L+). Table 10B also shows the percentage of total cells with effector memory phenotype (Tem; CD45RO+ CD62L− CCR7−). In addition to flow cytometry analysis, genomic DNA was prepared and NGS analysis performed as described in Example 1 to determine editing rates at each target site. Table 11 shows results for indel frequency at loci engineered in the third sequential edit.

TABLE 10A
Percentage of Vb8+CD8+ cells with stem cell memory phenotype

| | |
|---|---|
| % CD45RA+ | % CD45RA+ |
| CD62L+ CCR7+ | CD62L+ CCR7− |

| Sample | Mean | n | Mean | n |
|---|---|---|---|---|
| No guide | 16.2 | 1 | 83.8 | 1 |
| AAVS1 (G000562) | 44.4 | 1 | 56.5 | 1 |
| HD1 | 61.5 | 1 | 40.3 | 1 |
| PD1 (G018435) | 43.1 | 1 | 57.4 | 1 |

TABLE 10B
Percentage of Vb8+CD8+ cells with central memory cell
phenotype or with effector memory cell phenotype.

| | % CD45RO+ | | % CD45RO+ | | % CD45RO+ | |
|---|---|---|---|---|---|---|
| | CD62L+ CCR7+ | | CD62L+ CCR7− | | CD62L− CCR7− | |
| Sample | Mean | n | Mean | n | Mean | n |
| No guide | 13.3 | 1 | 86.7 | 1 | 95.6 | 1 |
| AAVS1 | 30.3 | 1 | 70.5 | 1 | 89.9 | 1 |
| (G000562) | | | | | | |
| HD1 | 49.4 | 1 | 71.0 | 1 | 90.7 | 1 |
| PD1 | 29.5 | 1 | 52.7 | 1 | 62.6 | 1 |
| (G018435) | | | | | | |

TABLE 11
Indel frequency at the PD1 loci

| | |
|---|---|
| Primer Set 1 | Primer Set 2 |

| Sample | Mean | n | Mean | n |
|---|---|---|---|---|
| G018435 [PD1] | 0.78 | 1 | 0.77 | 1 |

TABLE 12
Additional Sequences

| | | |
|---|---|---|
| G000562 | 107 | mC*mC*mA*AUAUCAGGAGACUAGGAGUUUUAGAmGmCm |
| | | UmAmGmAmAmAmUmAmGmCAAGUUAAAAUAAGGCUAGU |
| | | CCGUUAUCAmAmCmUmUmGmAmAmAmAmAmGmUmGmGm |
| | | CmAmCmCmGmAmGmUmCmGmGmUmGmCmU*mU*mU*mU |
| G000645 | 108 | mG*mA*mC*CCCCUCCACCCCGCCUCGUUUUAGAmGmCmU |
| | | mAmGmAmAmAmUmAmGmCAAGUUAAAAUAAGGCUAGUC |
| | | CGUUAUCAmAmCmUmUmGmAmAmAmAmAmGmUmGmGmC |
| | | mAmCmCmGmAmGmUmCmGmGmUmGmCmU*mU*mU*mU |
| G016239 | 109 | mG*mG*mC*CUCGGCGCUGACGAUCUGUUUUAGAmGmCmU |
| | | mAmGmAmAmAmUmAmGmCAAGUUAAAAUAAGGCUAGUC |
| | | CGUUAUCAmAmCmUmUmGmAmAmAmAmAmGmUmGmGmC |
| | | mAmCmCmGmAmGmUmCmGmGmUmGmCmU*mU*mU*mU |
| G013006 | 110 | mC*mU*mC*UCAGCUGGUACACGGCAGUUUUAGAmGmCmU |
| | | mAmGmAmAmAmUmAmGmCAAGUUAAAAUAAGGCUAGUC |
| | | CGUUAUCAmAmCmUmUmGmAmAmAmAmAmGmUmGmGmC |
| | | mAmCmCmGmAmGmUmCmGmGmUmGmCmU*mU*mU*mU |
| G018438 | 111 | mA*mG*mU*UGGGCAGAUAACACUUGGUUUUAGAmGmCm |
| | | UmAmGmAmAmAmUmAmGmCAAGUUAAAAUAAGGCUAGU |
| | | CCGUUAUCAmAmCmUmUmGmAmAmAmAmAmGmUmGmGm |
| | | CmAmCmCmGmAmGmUmCmGmGmUmGmCmU*mU*mU*mU |
| G000739 | 112 | mG*mA*mU*CACGUCGGCCGUUGGCGGUUUUAGAmGmCm |
| | | UmAmGmAmAmAmUmAmGmCAAGUUAAAAUAAGGCUAGU |
| | | CCGUUAUCAmAmCmUmUmGmAmAmAmAmAmGmUmGmGm |
| | | CmAmCmCmGmAmGmUmCmGmGmUmGmCmU*mU*mU*mU |
| G018435 | 113 | mC*mG*mA*CUGGCCAGGGCGCCUGUGUUUUAGAmGmCmU |
| | | mAmGmAmAmAmUmAmGmCAAGUUAAAAUAAGGCUAGUC |
| | | CGUUAUCAmAmCmUmUmGmAmAmAmAmAmGmUmGmGmC |
| | | mAmCmCmGmAmGmUmCmGmGmUmGmCmU*mU*mU*mU |
| G018432 | 114 | mC*mA*mC*CUACCUAAGAACCAUCCGUUUUAGAmGmCmU |
| | | mAmGmAmAmAmUmAmGmCAAGUUAAAAUAAGGCUAGUC |
| | | CGUUAUCAmAmCmUmUmGmAmAmAmAmAmGmUmGmGmC |
| | | mAmCmCmGmAmGmUmCmGmGmUmGmCmU*mU*mU*mU |
| CR003187 | 115 | GACCCCCUCCACCCCGCCUCGUUUUAGAGCUAUGCUGUU |
| | | UUG |
| tracrRNA | 204 | AACAGCAUAGCAAGUUAAAAUAAGGCUAGUCCGUUAUCA |
| | | ACUUGAAAAAGUGGCACCGAGUCGGUGCUUUUUUU |
| Recombinant | 800 | MDKKYSIGLDIGTNSVGWAVITDEYKVPSKKFKVLGNTDRHS |
| Cas9-NLS | | IKKNLIGALLFDSGETAEATRLKRTARRRYTRRKNRICYLQEIF |
| amino acid | | SNEMAKVDDSFFHRLEESFLVEEDKKHERHPIFGNIVDEVAYH |
| sequence | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDKV |
| | | MKQLKRRRYTGWGRLSRKLINGIRDKQSGKTILDFLKSDGFA |
| | | NRNFMQLIHDDSLTFKEDIQKAQVSGQGDSLHEHIANLAGSPA |
| | | IKKGILQTVKVVDELVKVMGRHKPENIVIEMARENQTTQKGQ |
| | | KNSRERMKRIEEGIKELGSQILKEHPVENTQLQNEKLYLYYLQ |
| | | NGRDMYVDQELDINRLSDYDVDHIVPQSFLKDDSIDNKVLTR |
| | | SDKNRGKSDNVPSEEVVKKMKNYWRQLLNAKLITQRKFDNL |
| | | TKAERGGLSELDKAGFIKRQLVETRQITKHVAQILDSRMNTKY |
| | | DENDKLIREVKVITLKSKLVSDFRKDFQFYKVREINNYHHAHD |
| | | AYLNAVVGTALIKKYPKLESEFVYGDYKVYDVRKMIAKSEQE |
| | | IGKATAKYFFYSNIMNFFKTEITLANGEIRKRPLIETNGETGEIV |
| | | WDKGRDFATVRKVLSMPQVNIVKKTEVQTGGFSKESILPKRN |
| | | SDKLIARKKDWDPKKYGGFDSPTVAYSVLVVAKVEKGKSKK |
| | | LKSVKELLGITIMERSSFEKNPIDFLEAKGYKEVKKDLIIKLPK |
| | | YSLFELENGRKRMLASAGELQKGNELALPSKYVNFLYLASHY |
| | | EKLKGSPEDNEQKQLFVEQHKHYLDEIIEQISEFSKRVILADAN |
| | | LDKVLSAYNKHRDKPIREQAENIIHLFTLTNLGAPAAFKYFDT |
| | | TIDRKRYTSTKEVLDATLIHQSITGLYETRIDLSQLGGDGGGSP |
| | | KKKRKV |
| ORF encoding | 801 | ATGGACAAGAAGTACAGCATCGGACTGGACATCGGAACAA |
| Sp. Cas9 | | ACAGCGTCGGATGGGCAGTCATCACAGACGAATACAAGGT |
| | | CCCGAGCAAGAAGTTCAAGGTCCTGGGAAACACAGACAGA |
| | | CACAGCATCAAGAAGAACCTGATCGGAGCACTGCTGTTCG |
| | | ACAGCGGAGAAACAGCAGAAGCAACAAGACTGAAGAGAA |
| | | CAGCAAGAAGAAGATACACAAGAAGAAAGAACAGAATCT |
| | | GCTACCTGCAGGAAATCTTCAGCAACGAAATGGCAAAGGT |
| | | CGACGACAGCTTCTTCCACAGACTGGAAGAAAGCTTCCTGG |
| | | TCGAAGAAGACAAGAAGCACGAAAGACACCCGATCTTCGG |
| | | AAACATCGTCGACGAAGTCGCATACCACGAAAAGTACCCG |
| | | ACAATCTACCACCTGAGAAAGAAGCTGGTCGACAGCACAG |
| | | ACAAGGCAGACCTGAGACTGATCTACCTGGCACTGGCACA |
| | | CATGATCAAGTTCAGAGGACACTTCCTGATCGAAGGAGAC |
| | | CTGAACCCGGACAACAGCGACGTCGACAAGCTGTTCATCC |
| | | AGCTGGTCCAGACATACAACCAGCTGTTCGAAGAAAACCC |
| | | GATCAACGCAAGCGGAGTCGACGCAAAGGCAATCCTGAGC |
| | | GCAAGACTGAGCAAGAGCAGAAGACTGGAAAACCTGATCG |
| | | CACAGCTGCCGGGAGAAAAGAAGAACGGACTGTTCGGAAA |
| | | CCTGATCGCACTGAGCCTGGGACTGACACCGAACTTCAAGA |
| | | GCAACTTCGACCTGGCAGAAGACGCAAAGCTGCAGCTGAG |
| | | CAAGGACACATACGACGACGACCTGGACAACCTGCTGGCA |
| | | CAGATCGGAGACCAGTACGCAGACCTGTTCCTGGCAGCAA |
| | | AGAACCTGAGCGACGCAATCCTGCTGAGCGACATCCTGAG |
| | | AGTCAACACAGAAATCACAAAGGCACCGCTGAGCGCAAGC |
| | | ATGATCAAGAGATACGACGAACACCACCAGGACCTGACAC |
| | | TGCTGAAGGCACTGGTCAGACAGCAGCTGCCGGAAAAGTA |
| | | CAAGGAAATCTTCTTCGACCAGAGCAAGAACGGATACGCA |
| | | GGATACATCGACGGAGGAGCAAGCCAGGAAGAATTCTACA |
| | | AGTTCATCAAGCCGATCCTGGAAAAGATGGACGGAACAGA |
| | | AGAACTGCTGGTCAAGCTGAACAGAGAAGACCTGCTGAGA |
| | | AAGCAGAGAACATTCGACAACGGAAGCATCCCGCACCAGA |
| | | TCCACCTGGGAGAACTGCACGCAATCCTGAGAAGACAGGA |
| | | AGACTTCTACCCGTTCCTGAAGGACAACAGAGAAAAGATC |
| | | GAAAAGATCCTGACATTCAGAATCCCGTACTACGTCGGACC |
| | | GCTGGCAAGAGGAAACAGCAGATTCGCATGGATGACAAGA |
| | | AAGAGCGAAGAAACAATCACACCGTGGAACTTCGAAGAAG |
| | | TCGTCGACAAGGGAGCAAGCGCACAGAGCTTCATCGAAAG |
| | | AATGACAAACTTCGACAAGAACCTGCCGAACGAAAAGGTC |
| | | CTGCCGAAGCACAGCCTGCTGTACGAATACTTCACAGTCTA |
| | | CAACGAACTGACAAAGGTCAAGTACGTCACAGAAGGAATG |
| | | AGAAAGCCGGCATTCCTGAGCGGAGAACAGAAGAAGGCAA |
| | | TCGTCGACCTGCTGTTCAAGACAAACAGAAAGGTCACAGTC |
| | | AAGCAGCTGAAGGAAGACTACTTCAAGAAGATCGAATGCT |
| | | TCGACAGCGTCGAAATCAGCGGAGTCGAAGACAGATTCAA |
| | | CGCAAGCCTGGGAACATACCACGACCTGCTGAAGATCATC |
| | | AAGGACAAGGACTTCCTGGACAACGAAGAAAACGAAGACA |
| | | TCCTGGAAGACATCGTCCTGACACTGACACTGTTCGAAGAC |
| | | AGAGAAATGATCGAAGAAAGACTGAAGACATACGCACACC |
| | | TGTTCGACGACAAGGTCATGAAGCAGCTGAAGAGAAGAAG |
| | | ATACACAGGATGGGGAAGACTGAGCAGAAAGCTGATCAAC |
| | | GGAATCAGAGACAAGCAGAGCGGAAAGACAATCCTGGACT |
| | | TCCTGAAGAGCGACGGATTCGCAAACAGAAACTTCATGCA |
| | | GCTGATCCACGACGACAGCCTGACATTCAAGGAAGACATC |
| | | CAGAAGGCACAGGTCAGCGGACAGGGAGACAGCCTGCACG |
| | | AACACATCGCAAACCTGGCAGGAAGCCCGGCAATCAAGAA |
| | | GGGAATCCTGCAGACAGTCAAGGTCGTCGACGAACTGGTC |
| | | AAGGTCATGGGAAGACACAAGCCGGAAAACATCGTCATCG |
| | | AAATGGCAAGAGAAAACCAGACAACACAGAAGGGACAGA |
| | | AGAACAGCAGAGAAAGAATGAAGAGAATCGAAGAAGGAA |
| | | TCAAGGAACTGGGAAGCCAGATCCTGAAGGAACACCCGGT |
| | | CGAAAACACACAGCTGCAGAACGAAAAGCTGTACCTGTAC |
| | | TACCTGCAGAACGGAAGAGACATGTACGTCGACCAGGAAC |
| | | TGGACATCAACAGACTGAGCGACTACGACGTCGACCACAT |
| | | CGTCCCGCAGAGCTTCCTGAAGGACGACAGCATCGACAAC |
| | | AAGGTCCTGACAAGAAGCGACAAGAACAGAGGAAAGAGC |
| | | GACAACGTCCCGAGCGAAGAAGTCGTCAAGAAGATGAAGA |
| | | ACTACTGGAGACAGCTGCTGAACGCAAAGCTGATCACACA |
| | | GAGAAAGTTCGACAACCTGACAAAGGCAGAGAGAGGAGG |
| | | ACTGAGCGAACTGGACAAGGCAGGATTCATCAAGAGACAG |
| | | CTGGTCGAAACAAGACAGATCACAAAGCACGTCGCACAGA |
| | | TCCTGGACAGCAGAATGAACACAAAGTACGACGAAAACGA |
| | | CAAGCTGATCAGAGAAGTCAAGGTCATCACACTGAAGAGC |
| | | AAGCTGGTCAGCGACTTCAGAAAGGACTTCCAGTTCTACAA |
| | | GGTCAGAGAAATCAACAACTACCACCACGCACACGACGCA |
| | | TACCTGAACGCAGTCGTCGGAACAGCACTGATCAAGAAGT |
| | | ACCCGAAGCTGGAAAGCGAATTCGTCTACGGAGACTACAA |
| | | GGTCTACGACGTCAGAAAGATGATCGCAAAGAGCGAACAG |
| | | GAAATCGGAAAGGCAACAGCAAAGTACTTCTTCTACAGCA |
| | | ACATCATGAACTTCTTCAAGACAGAAATCACACTGGCAAAC |
| | | GGAGAAATCAGAAAGAGACCGCTGATCGAAACAAACGGA |
| | | GAAACAGGAGAAATCGTCTGGGACAAGGGAAGAGACTTCG |
| | | CAACAGTCAGAAAGGTCCTGAGCATGCCGCAGGTCAACAT |
| | | CGTCAAGAAGACAGAAGTCCAGACAGGAGGATTCAGCAAG |
| | | GAAAGCATCCTGCCGAAGAGAAACAGCGACAAGCTGATCG |
| | | CAAGAAAGAAGGACTGGGACCCGAAGAAGTACGGAGGATT |
| | | CGACAGCCCGACAGTCGCATACAGCGTCCTGGTCGTCGCAA |
| | | AGGTCGAAAAGGGAAAGAGCAAGAAGCTGAAGAGCGTCA |
| | | AGGAACTGCTGGGAATCACAATCATGGAAAGAAGCAGCTT |
| | | CGAAAAGAACCCGATCGACTTCCTGGAAGCAAAGGGATAC |
| | | AAGGAAGTCAAGAAGGACCTGATCATCAAGCTGCCGAAGT |
| | | ACAGCCTGTTCGAACTGGAAAACGGAAGAAAGAGAATGCT |
| | | GGCAAGCGCAGGAGAACTGCAGAAGGGAAACGAACTGGC |
| | | ACTGCCGAGCAAGTACGTCAACTTCCTGTACCTGGCAAGCC |
| | | ACTACGAAAAGCTGAAGGGAAGCCCGGAAGACAACGAAC |
| | | AGAAGCAGCTGTTCGTCGAACAGCACAAGCACTACCTGGA |
| | | CGAAATCATCGAACAGATCAGCGAATTCAGCAAGAGAGTC |
| | | ATCCTGGCAGACGCAAACCTGGACAAGGTCCTGAGCGCAT |
| | | ACAACAAGCACAGAGACAAGCCGATCAGAGAACAGGCAG |
| | | AAAACATCATCCACCTGTTCACACTGACAAACCTGGGAGCA |
| | | CCGGCAGCATTCAAGTACTTCGACACAACAATCGACAGAA |
| | | AGAGATACACAAGCACAAAGGAAGTCCTGGACGCAACACT |
| | | GATCCACCAGAGCATCACAGGACTGTACGAAACAAGAATC |
| | | GACCTGAGCCAGCTGGGAGGAGACGGAGGAGGAAGCCCG |
| | | AAGAAGAAGAGAAAGGTCTAG |
| ORF encoding | 802 | ATGGACAAGAAGTACTCCATCGGCCTGGACATCGGCACCA |
| Sp. Cas9 | | ACTCCGTGGGCTGGGCCGTGATCACCGACGAGTACAAGGT |
| | | GCCCTCCAAGAAGTTCAAGGTGCTGGGCAACACCGACCGG |
| | | CACTCCATCAAGAAGAACCTGATCGGCGCCCTGCTGTTCGA |
| | | CTCCGGCGAGACCGCCGAGGCCACCCGGCTGAAGCGGACC |
| | | GCCCGGCGGCGGTACACCCGGCGGAAGAACCGGATCTGCT |
| | | ACCTGCAGGAGATCTTCTCCAACGAGATGGCCAAGGTGGA |
| | | CGACTCCTTCTTCCACCGGCTGGAGGAGTCCTTCCTGGTGG |
| | | AGGAGGACAAGAAGCACGAGCGGCACCCCATCTTCGGCAA |
| | | CATCGTGGACGAGGTGGCCTACCACGAGAAGTACCCCACC |
| | | ATCTACCACCTGCGGAAGAAGCTGGTGGACTCCACCGACA |
| | | AGGCCGACCTGCGGCTGATCTACCTGGCCCTGGCCCACATG |
| | | ATCAAGTTCCGGGGCCACTTCCTGATCGAGGGCGACCTGAA |
| | | CCCCGACAACTCCGACGTGGACAAGCTGTTCATCCAGCTGG |
| | | TGCAGACCTACAACCAGCTGTTCGAGGAGAACCCCATCAA |
| | | CGCCTCCGGCGTGGACGCCAAGGCCATCCTGTCCGCCCGGC |
| | | TGTCCAAGTCCCGGCGGCTGGAGAACCTGATCGCCCAGCTG |
| | | CCCGGCGAGAAGAAGAACGGCCTGTTCGGCAACCTGATCG |
| | | CCCTGTCCCTGGGCCTGACCCCCAACTTCAAGTCCAACTTC |
| | | GACCTGGCCGAGGACGCCAAGCTGCAGCTGTCCAAGGACA |
| | | CCTACGACGACGACCTGGACAACCTGCTGGCCCAGATCGG |
| | | CGACCAGTACGCCGACCTGTTCCTGGCCGCCAAGAACCTGT |
| | | CCGACGCCATCCTGCTGTCCGACATCCTGCGGGTGAACACC |
| | | GAGATCACCAAGGCCCCCCTGTCCGCCTCCATGATCAAGCG |
| | | GTACGACGAGCACCACCAGGACCTGACCCTGCTGAAGGCC |
| | | CTGGTGCGGCAGCAGCTGCCCGAGAAGTACAAGGAGATCT |
| | | TCTTCGACCAGTCCAAGAACGGCTACGCCGGCTACATCGAC |
| | | GGCGGCGCCTCCCAGGAGGAGTTCTACAAGTTCATCAAGCC |
| | | CATCCTGGAGAAGATGGACGGCACCGAGGAGCTGCTGGTG |
| | | AAGCTGAACCGGGAGGACCTGCTGCGGAAGCAGCGGACCT |
| | | TCGACAACGGCTCCATCCCCCACCAGATCCACCTGGGCGAG |
| | | CTGCACGCCATCCTGCGGCGGCAGGAGGACTTCTACCCCTT |
| | | CCTGAAGGACAACCGGGAGAAGATCGAGAAGATCCTGACC |
| | | TTCCGGATCCCCTACTACGTGGGCCCCCTGGCCCGGGGCAA |
| | | CTCCCGGTTCGCCTGGATGACCCGGAAGTCCGAGGAGACC |
| | | ATCACCCCCTGGAACTTCGAGGAGGTGGTGGACAAGGGCG |
| | | CCTCCGCCCAGTCCTTCATCGAGCGGATGACCAACTTCGAC |
| | | AAGAACCTGCCCAACGAGAAGGTGCTGCCCAAGCACTCCC |
| | | TGCTGTACGAGTACTTCACCGTGTACAACGAGCTGACCAAG |
| | | GTGAAGTACGTGACCGAGGGCATGCGGAAGCCCGCCTTCC |
| | | TGTCCGGCGAGCAGAAGAAGGCCATCGTGGACCTGCTGTTC |
| | | AAGACCAACCGGAAGGTGACCGTGAAGCAGCTGAAGGAGG |
| | | ACTACTTCAAGAAGATCGAGTGCTTCGACTCCGTGGAGATC |
| | | TCCGGCGTGGAGGACCGGTTCAACGCCTCCCTGGGCACCTA |
| | | CCACGACCTGCTGAAGATCATCAAGGACAAGGACTTCCTG |
| | | GACAACGAGGAGAACGAGGACATCCTGGAGGACATCGTGC |
| | | TGACCCTGACCCTGTTCGAGGACCGGGAGATGATCGAGGA |
| | | GCGGCTGAAGACCTACGCCCACCTGTTCGACGACAAGGTG |
| | | ATGAAGCAGCTGAAGCGGCGGCGGTACACCGGCTGGGGCC |
| | | GGCTGTCCCGGAAGCTGATCAACGGCATCCGGGACAAGCA |
| | | GTCCGGCAAGACCATCCTGGACTTCCTGAAGTCCGACGGCT |
| | | TCGCCAACCGGAACTTCATGCAGCTGATCCACGACGACTCC |
| | | CTGACCTTCAAGGAGGACATCCAGAAGGCCCAGGTGTCCG |
| | | GCCAGGGCGACTCCCTGCACGAGCACATCGCCAACCTGGC |
| | | CGGCTCCCCCGCCATCAAGAAGGGCATCCTGCAGACCGTG |
| | | AAGGTGGTGGACGAGCTGGTGAAGGTGATGGGCCGGCACA |
| | | AGCCCGAGAACATCGTGATCGAGATGGCCCGGGAGAACCA |
| | | GACCACCCAGAAGGGCCAGAAGAACTCCCGGGAGCGGATG |
| | | AAGCGGATCGAGGAGGGCATCAAGGAGCTGGGCTCCCAGA |
| | | TCCTGAAGGAGCACCCCGTGGAGAACACCCAGCTGCAGAA |
| | | CGAGAAGCTGTACCTGTACTACCTGCAGAACGGCCGGGAC |
| | | ATGTACGTGGACCAGGAGCTGGACATCAACCGGCTGTCCG |
| | | ACTACGACGTGGACCACATCGTGCCCCAGTCCTTCCTGAAG |
| | | GACGACTCCATCGACAACAAGGTGCTGACCCGGTCCGACA |
| | | AGAACCGGGGCAAGTCCGACAACGTGCCCTCCGAGGAGGT |
| | | GGTGAAGAAGATGAAGAACTACTGGCGGCAGCTGCTGAAC |
| | | GCCAAGCTGATCACCCAGCGGAAGTTCGACAACCTGACCA |
| | | AGGCCGAGCGGGGCGGCCTGTCCGAGCTGGACAAGGCCGG |
| | | CTTCATCAAGCGGCAGCTGGTGGAGACCCGGCAGATCACC |
| | | AAGCACGTGGCCCAGATCCTGGACTCCCGGATGAACACCA |
| | | AGTACGACGAGAACGACAAGCTGATCCGGGAGGTGAAGGT |
| | | GATCACCCTGAAGTCCAAGCTGGTGTCCGACTTCCGGAAGG |
| | | ACTTCCAGTTCTACAAGGTGCGGGAGATCAACAACTACCAC |
| | | CACGCCCACGACGCCTACCTGAACGCCGTGGTGGGCACCG |
| | | CCCTGATCAAGAAGTACCCCAAGCTGGAGTCCGAGTTCGTG |
| | | TACGGCGACTACAAGGTGTACGACGTGCGGAAGATGATCG |
| | | CCAAGTCCGAGCAGGAGATCGGCAAGGCCACCGCCAAGTA |
| | | CTTCTTCTACTCCAACATCATGAACTTCTTCAAGACCGAGA |
| | | TCACCCTGGCCAACGGCGAGATCCGGAAGCGGCCCCTGAT |
| | | CGAGACCAACGGCGAGACCGGCGAGATCGTGTGGGACAAG |
| | | GGCCGGGACTTCGCCACCGTGCGGAAGGTGCTGTCCATGCC |
| | | CCAGGTGAACATCGTGAAGAAGACCGAGGTGCAGACCGGC |
| | | GGCTTCTCCAAGGAGTCCATCCTGCCCAAGCGGAACTCCGA |
| | | CAAGCTGATCGCCCGGAAGAAGGACTGGGACCCCAAGAAG |
| | | TACGGCGGCTTCGACTCCCCCACCGTGGCCTACTCCGTGCT |
| | | GGTGGTGGCCAAGGTGGAGAAGGGCAAGTCCAAGAAGCTG |
| | | AAGTCCGTGAAGGAGCTGCTGGGCATCACCATCATGGAGC |
| | | GGTCCTCCTTCGAGAAGAACCCCATCGACTTCCTGGAGGCC |
| | | AAGGGCTACAAGGAGGTGAAGAAGGACCTGATCATCAAGC |
| | | TGCCCAAGTACTCCCTGTTCGAGCTGGAGAACGGCCGGAA |
| | | GCGGATGCTGGCCTCCGCCGGCGAGCTGCAGAAGGGCAAC |
| | | GAGCTGGCCCTGCCCTCCAAGTACGTGAACTTCCTGTACCT |
| | | GGCCTCCCACTACGAGAAGCTGAAGGGCTCCCCCGAGGAC |
| | | AACGAGCAGAAGCAGCTGTTCGTGGAGCAGCACAAGCACT |
| | | ACCTGGACGAGATCATCGAGCAGATCTCCGAGTTCTCCAAG |
| | | CGGGTGATCCTGGCCGACGCCAACCTGGACAAGGTGCTGTC |
| | | CGCCTACAACAAGCACCGGGACAAGCCCATCCGGGAGCAG |
| | | GCCGAGAACATCATCCACCTGTTCACCCTGACCAACCTGGG |
| | | CGCCCCCGCCGCCTTCAAGTACTTCGACACCACCATCGACC |
| | | GGAAGCGGTACACCTCCACCAAGGAGGTGCTGGACGCCAC |
| | | CCTGATCCACCAGTCCATCACCGGCCTGTACGAGACCCGGA |
| | | TCGACCTGTCCCAGCTGGGCGGCGACGGCGGCGGCTCCCCC |
| | | AAGAAGAAGCGGAAGGTGTGA |
| Open reading | 803 | AUGGACAAGAAGUACUCCAUCGGCCUGGACAUCGGCACC |
| frame for Cas9 | | AACUCCGUGGGCUGGGCCGUGAUCACCGACGAGUACAAG |
| with Hibit tag | | GUGCCCUCCAAGAAGUUCAAGGUGCUGGGCAACACCGAC |
| | | CGGCACUCCAUCAAGAAGAACCUGAUCGGCGCCCUGCUGU |
| | | UCGACUCCGGCGAGACCGCCGAGGCCACCCGGCUGAAGCG |
| | | GACCGCCCGGCGGCGGUACACCCGGCGGAAGAACCGGAUC |
| | | UGCUACCUGCAGGAGAUCUUCUCCAACGAGAUGGCCAAG |
| | | GUGGACGACUCCUUCUUCCACCGGCUGGAGGAGUCCUUCC |
| | | UGGUGGAGGAGGACAAGAAGCACGAGCGGCACCCCAUCU |
| | | UCGGCAACAUCGUGGACGAGGUGGCCUACCACGAGAAGU |
| | | ACCCCACCAUCUACCACCUGCGGAAGAAGCUGGUGGACUC |
| | | CACCGACAAGGCCGACCUGCGGCUGAUCUACCUGGCCCUG |
| | | GCCCACAUGAUCAAGUUCCGGGGCCACUUCCUGAUCGAG |
| | | GGCGACCUGAACCCCGACAACUCCGACGUGGACAAGCUGU |
| | | UCAUCCAGCUGGUGCAGACCUACAACCAGCUGUUCGAGG |
| | | AGAACCCCAUCAACGCCUCCGGCGUGGACGCCAAGGCCAU |
| | | CCUGUCCGCCCGGCUGUCCAAGUCCCGGCGGCUGGAGAAC |
| | | CUGAUCGCCCAGCUGCCCGGCGAGAAGAAGAACGGCCUG |
| | | UUCGGCAACCUGAUCGCCCUGUCCCUGGGCCUGACCCCCA |
| | | ACUUCAAGUCCAACUUCGACCUGGCCGAGGACGCCAAGCU |
| | | GCAGCUGUCCAAGGACACCUACGACGACGACCUGGACAAC |
| | | CUGCUGGCCCAGAUCGGCGACCAGUACGCCGACCUGUUCC |
| | | UGGCCGCCAAGAACCUGUCCGACGCCAUCCUGCUGUCCGA |
| | | CAUCCUGCGGGUGAACACCGAGAUCACCAAGGCCCCCCUG |
| | | UCCGCCUCCAUGAUCAAGCGGUACGACGAGCACCACCAGG |
| | | ACCUGACCCUGCUGAAGGCCCUGGUGCGGCAGCAGCUGCC |
| | | CGAGAAGUACAAGGAGAUCUUCUUCGACCAGUCCAAGAA |
| | | CGGCUACGCCGGCUACAUCGACGGCGGCGCCUCCCAGGAG |
| | | GAGUUCUACAAGUUCAUCAAGCCCAUCCUGGAGAAGAUG |
| | | GACGGCACCGAGGAGCUGCUGGUGAAGCUGAACCGGGAG |
| | | GACCUGCUGCGGAAGCAGCGGACCUUCGACAACGGCUCCA |
| | | UCCCCCACCAGAUCCACCUGGGCGAGCUGCACGCCAUCCU |
| | | GCGGCGGCAGGAGGACUUCUACCCCUUCCUGAAGGACAA |
| | | CCGGGAGAAGAUCGAGAAGAUCCUGACCUUCCGGAUCCC |
| | | CUACUACGUGGGCCCCCUGGCCCGGGGCAACUCCCGGUUC |
| | | GCCUGGAUGACCCGGAAGUCCGAGGAGACCAUCACCCCCU |
| | | GGAACUUCGAGGAGGUGGUGGACAAGGGCGCCUCCGCCC |
| | | AGUCCUUCAUCGAGCGGAUGACCAACUUCGACAAGAACC |
| | | UGCCCAACGAGAAGGUGCUGCCCAAGCACUCCCUGCUGUA |
| | | CGAGUACUUCACCGUGUACAACGAGCUGACCAAGGUGAA |
| | | GUACGUGACCGAGGGCAUGCGGAAGCCCGCCUUCCUGUCC |
| | | GGCGAGCAGAAGAAGGCCAUCGUGGACCUGCUGUUCAAG |
| | | ACCAACCGGAAGGUGACCGUGAAGCAGCUGAAGGAGGAC |
| | | UACUUCAAGAAGAUCGAGUGCUUCGACUCCGUGGAGAUC |
| | | UCCGGCGUGGAGGACCGGUUCAACGCCUCCCUGGGCACCU |
| | | ACCACGACCUGCUGAAGAUCAUCAAGGACAAGGACUUCC |
| | | UGGACAACGAGGAGAACGAGGACAUCCUGGAGGACAUCG |
| | | UGCUGACCCUGACCCUGUUCGAGGACCGGGAGAUGAUCG |
| | | AGGAGCGGCUGAAGACCUACGCCCACCUGUUCGACGACA |
| | | AGGUGAUGAAGCAGCUGAAGCGGCGGCGGUACACCGGCU |
| | | GGGGCCGGCUGUCCCGGAAGCUGAUCAACGGCAUCCGGG |
| | | ACAAGCAGUCCGGCAAGACCAUCCUGGACUUCCUGAAGU |
| | | CCGACGGCUUCGCCAACCGGAACUUCAUGCAGCUGAUCCA |
| | | CGACGACUCCCUGACCUUCAAGGAGGACAUCCAGAAGGCC |
| | | CAGGUGUCCGGCCAGGGCGACUCCCUGCACGAGCACAUCG |
| | | CCAACCUGGCCGGCUCCCCCGCCAUCAAGAAGGGCAUCCU |
| | | GCAGACCGUGAAGGUGGUGGACGAGCUGGUGAAGGUGAU |
| | | GGGCCGGCACAAGCCCGAGAACAUCGUGAUCGAGAUGGC |
| | | CCGGGAGAACCAGACCACCCAGAAGGGCCAGAAGAACUCC |
| | | CGGGAGCGGAUGAAGCGGAUCGAGGAGGGCAUCAAGGAG |
| | | CUGGGCUCCCAGAUCCUGAAGGAGCACCCCGUGGAGAAC |
| | | ACCCAGCUGCAGAACGAGAAGCUGUACCUGUACUACCUG |
| | | CAGAACGGCCGGGACAUGUACGUGGACCAGGAGCUGGAC |
| | | AUCAACCGGCUGUCCGACUACGACGUGGACCACAUCGUGC |
| | | CCCAGUCCUUCCUGAAGGACGACUCCAUCGACAACAAGGU |
| | | GCUGACCCGGUCCGACAAGAACCGGGGCAAGUCCGACAAC |
| | | GUGCCCUCCGAGGAGGUGGUGAAGAAGAUGAAGAACUAC |
| | | UGGCGGCAGCUGCUGAACGCCAAGCUGAUCACCCAGCGG |
| | | AAGUUCGACAACCUGACCAAGGCCGAGCGGGGCGGCCUG |
| | | UCCGAGCUGGACAAGGCCGGCUUCAUCAAGCGGCAGCUG |
| | | GUGGAGACCCGGCAGAUCACCAAGCACGUGGCCCAGAUCC |
| | | UGGACUCCCGGAUGAACACCAAGUACGACGAGAACGACA |
| | | AGCUGAUCCGGGAGGUGAAGGUGAUCACCCUGAAGUCCA |
| | | AGCUGGUGUCCGACUUCCGGAAGGACUUCCAGUUCUACA |
| | | AGGUGCGGGAGAUCAACAACUACCACCACGCCCACGACGC |
| | | CUACCUGAACGCCGUGGUGGGCACCGCCCUGAUCAAGAA |
| | | GUACCCCAAGCUGGAGUCCGAGUUCGUGUACGGCGACUA |
| | | CAAGGUGUACGACGUGCGGAAGAUGAUCGCCAAGUCCGA |
| | | GCAGGAGAUCGGCAAGGCCACCGCCAAGUACUUCUUCUA |
| | | CUCCAACAUCAUGAACUUCUUCAAGACCGAGAUCACCCUG |
| | | GCCAACGGCGAGAUCCGGAAGCGGCCCCUGAUCGAGACCA |
| | | ACGGCGAGACCGGCGAGAUCGUGUGGGACAAGGGCCGGG |
| | | ACUUCGCCACCGUGCGGAAGGUGCUGUCCAUGCCCCAGGU |
| | | GAACAUCGUGAAGAAGACCGAGGUGCAGACCGGCGGCUU |
| | | CUCCAAGGAGUCCAUCCUGCCCAAGCGGAACUCCGACAAG |
| | | CUGAUCGCCCGGAAGAAGGACUGGGACCCCAAGAAGUAC |
| | | GGCGGCUUCGACUCCCCCACCGUGGCCUACUCCGUGCUGG |
| | | UGGUGGCCAAGGUGGAGAAGGGCAAGUCCAAGAAGCUGA |
| | | AGUCCGUGAAGGAGCUGCUGGGCAUCACCAUCAUGGAGC |
| | | GGUCCUCCUUCGAGAAGAACCCCAUCGACUUCCUGGAGGC |
| | | CAAGGGCUACAAGGAGGUGAAGAAGGACCUGAUCAUCAA |
| | | GCUGCCCAAGUACUCCCUGUUCGAGCUGGAGAACGGCCG |
| | | GAAGCGGAUGCUGGCCUCCGCCGGCGAGCUGCAGAAGGG |
| | | CAACGAGCUGGCCCUGCCCUCCAAGUACGUGAACUUCCUG |
| | | UACCUGGCCUCCCACUACGAGAAGCUGAAGGGCUCCCCCG |
| | | AGGACAACGAGCAGAAGCAGCUGUUCGUGGAGCAGCACA |
| | | AGCACUACCUGGACGAGAUCAUCGAGCAGAUCUCCGAGU |
| | | UCUCCAAGCGGGUGAUCCUGGCCGACGCCAACCUGGACAA |
| | | GGUGCUGUCCGCCUACAACAAGCACCGGGACAAGCCCAUC |
| | | CGGGAGCAGGCCGAGAACAUCAUCCACCUGUUCACCCUGA |
| | | CCAACCUGGGCGCCCCCGCCGCCUUCAAGUACUUCGACAC |
| | | CACCAUCGACCGGAAGCGGUACACCUCCACCAAGGAGGUG |
| | | CUGGACGCCACCCUGAUCCACCAGUCCAUCACCGGCCUGU |
| | | ACGAGACCCGGAUCGACCUGUCCCAGCUGGGCGGCGACGG |
| | | CGGCGGCUCCCCCAAGAAGAAGCGGAAGGUGUCCGAGUC |
| | | CGCCACCCCCGAGUCCGUGUCCGGCUGGCGGCUGUUCAAG |
| | | AAGAUCUCCUGA |
| RNA encoding | 804 | AUGGACAAGAAGUACUCCAUCGGCCUGGACAUCGGCACC |
| Cas9 | | AACUCCGUGGGCUGGGCCGUGAUCACCGACGAGUACAAG |
| corresponding | | GUGCCCUCCAAGAAGUUCAAGGUGCUGGGCAACACCGAC |
| to SEQ ID NO: | | CGGCACUCCAUCAAGAAGAACCUGAUCGGCGCCCUGCUGU |
| 802 | | UCGACUCCGGCGAGACCGCCGAGGCCACCCGGCUGAAGCG |
| | | GACCGCCCGGCGGCGGUACACCCGGCGGAAGAACCGGAUC |
| | | UGCUACCUGCAGGAGAUCUUCUCCAACGAGAUGGCCAAG |
| | | GUGGACGACUCCUUCUUCCACCGGCUGGAGGAGUCCUUCC |
| | | UGGUGGAGGAGGACAAGAAGCACGAGCGGCACCCCAUCU |
| | | UCGGCAACAUCGUGGACGAGGUGGCCUACCACGAGAAGU |
| | | ACCCCACCAUCUACCACCUGCGGAAGAAGCUGGUGGACUC |
| | | CACCGACAAGGCCGACCUGCGGCUGAUCUACCUGGCCCUG |
| | | GCCCACAUGAUCAAGUUCCGGGGCCACUUCCUGAUCGAG |
| | | GGCGACCUGAACCCCGACAACUCCGACGUGGACAAGCUGU |
| | | UCAUCCAGCUGGUGCAGACCUACAACCAGCUGUUCGAGG |
| | | AGAACCCCAUCAACGCCUCCGGCGUGGACGCCAAGGCCAU |
| | | CCUGUCCGCCCGGCUGUCCAAGUCCCGGCGGCUGGAGAAC |
| | | CUGAUCGCCCAGCUGCCCGGCGAGAAGAAGAACGGCCUG |
| | | UUCGGCAACCUGAUCGCCCUGUCCCUGGGCCUGACCCCCA |
| | | ACUUCAAGUCCAACUUCGACCUGGCCGAGGACGCCAAGCU |
| | | GCAGCUGUCCAAGGACACCUACGACGACGACCUGGACAAC |
| | | CUGCUGGCCCAGAUCGGCGACCAGUACGCCGACCUGUUCC |
| | | UGGCCGCCAAGAACCUGUCCGACGCCAUCCUGCUGUCCGA |
| | | CAUCCUGCGGGUGAACACCGAGAUCACCAAGGCCCCCCUG |
| | | UCCGCCUCCAUGAUCAAGCGGUACGACGAGCACCACCAGG |
| | | ACCUGACCCUGCUGAAGGCCCUGGUGCGGCAGCAGCUGCC |
| | | CGAGAAGUACAAGGAGAUCUUCUUCGACCAGUCCAAGAA |
| | | CGGCUACGCCGGCUACAUCGACGGCGGCGCCUCCCAGGAG |
| | | GAGUUCUACAAGUUCAUCAAGCCCAUCCUGGAGAAGAUG |
| | | GACGGCACCGAGGAGCUGCUGGUGAAGCUGAACCGGGAG |
| | | GACCUGCUGCGGAAGCAGCGGACCUUCGACAACGGCUCCA |
| | | UCCCCCACCAGAUCCACCUGGGCGAGCUGCACGCCAUCCU |
| | | GCGGCGGCAGGAGGACUUCUACCCCUUCCUGAAGGACAA |
| | | CCGGGAGAAGAUCGAGAAGAUCCUGACCUUCCGGAUCCC |
| | | CUACUACGUGGGCCCCCUGGCCCGGGGCAACUCCCGGUUC |
| | | GCCUGGAUGACCCGGAAGUCCGAGGAGACCAUCACCCCCU |
| | | GGAACUUCGAGGAGGUGGUGGACAAGGGCGCCUCCGCCC |
| | | AGUCCUUCAUCGAGCGGAUGACCAACUUCGACAAGAACC |
| | | UGCCCAACGAGAAGGUGCUGCCCAAGCACUCCCUGCUGUA |
| | | CGAGUACUUCACCGUGUACAACGAGCUGACCAAGGUGAA |
| | | GUACGUGACCGAGGGCAUGCGGAAGCCCGCCUUCCUGUCC |
| | | GGCGAGCAGAAGAAGGCCAUCGUGGACCUGCUGUUCAAG |
| | | ACCAACCGGAAGGUGACCGUGAAGCAGCUGAAGGAGGAC |
| | | UACUUCAAGAAGAUCGAGUGCUUCGACUCCGUGGAGAUC |
| | | UCCGGCGUGGAGGACCGGUUCAACGCCUCCCUGGGCACCU |
| | | ACCACGACCUGCUGAAGAUCAUCAAGGACAAGGACUUCC |
| | | UGGACAACGAGGAGAACGAGGACAUCCUGGAGGACAUCG |
| | | UGCUGACCCUGACCCUGUUCGAGGACCGGGAGAUGAUCG |
| | | AGGAGCGGCUGAAGACCUACGCCCACCUGUUCGACGACA |
| | | AGGUGAUGAAGCAGCUGAAGCGGCGGCGGUACACCGGCU |
| | | GGGGCCGGCUGUCCCGGAAGCUGAUCAACGGCAUCCGGG |
| | | ACAAGCAGUCCGGCAAGACCAUCCUGGACUUCCUGAAGU |
| | | CCGACGGCUUCGCCAACCGGAACUUCAUGCAGCUGAUCCA |
| | | CGACGACUCCCUGACCUUCAAGGAGGACAUCCAGAAGGCC |
| | | CAGGUGUCCGGCCAGGGCGACUCCCUGCACGAGCACAUCG |
| | | CCAACCUGGCCGGCUCCCCCGCCAUCAAGAAGGGCAUCCU |
| | | GCAGACCGUGAAGGUGGUGGACGAGCUGGUGAAGGUGAU |
| | | GGGCCGGCACAAGCCCGAGAACAUCGUGAUCGAGAUGGC |
| | | CCGGGAGAACCAGACCACCCAGAAGGGCCAGAAGAACUCC |
| | | CGGGAGCGGAUGAAGCGGAUCGAGGAGGGCAUCAAGGAG |
| | | CUGGGCUCCCAGAUCCUGAAGGAGCACCCCGUGGAGAAC |
| | | ACCCAGCUGCAGAACGAGAAGCUGUACCUGUACUACCUG |
| | | CAGAACGGCCGGGACAUGUACGUGGACCAGGAGCUGGAC |
| | | AUCAACCGGCUGUCCGACUACGACGUGGACCACAUCGUGC |
| | | CCCAGUCCUUCCUGAAGGACGACUCCAUCGACAACAAGGU |
| | | GCUGACCCGGUCCGACAAGAACCGGGGCAAGUCCGACAAC |
| | | GUGCCCUCCGAGGAGGUGGUGAAGAAGAUGAAGAACUAC |
| | | UGGCGGCAGCUGCUGAACGCCAAGCUGAUCACCCAGCGG |
| | | AAGUUCGACAACCUGACCAAGGCCGAGCGGGGCGGCCUG |
| | | UCCGAGCUGGACAAGGCCGGCUUCAUCAAGCGGCAGCUG |
| | | GUGGAGACCCGGCAGAUCACCAAGCACGUGGCCCAGAUCC |
| | | UGGACUCCCGGAUGAACACCAAGUACGACGAGAACGACA |
| | | AGCUGAUCCGGGAGGUGAAGGUGAUCACCCUGAAGUCCA |
| | | AGCUGGUGUCCGACUUCCGGAAGGACUUCCAGUUCUACA |
| | | AGGUGCGGGAGAUCAACAACUACCACCACGCCCACGACGC |
| | | CUACCUGAACGCCGUGGUGGGCACCGCCCUGAUCAAGAA |
| | | GUACCCCAAGCUGGAGUCCGAGUUCGUGUACGGCGACUA |
| | | CAAGGUGUACGACGUGCGGAAGAUGAUCGCCAAGUCCGA |
| | | GCAGGAGAUCGGCAAGGCCACCGCCAAGUACUUCUUCUA |
| | | CUCCAACAUCAUGAACUUCUUCAAGACCGAGAUCACCCUG |
| | | GCCAACGGCGAGAUCCGGAAGCGGCCCCUGAUCGAGACCA |
| | | ACGGCGAGACCGGCGAGAUCGUGUGGGACAAGGGCCGGG |
| | | ACUUCGCCACCGUGCGGAAGGUGCUGUCCAUGCCCCAGGU |
| | | GAACAUCGUGAAGAAGACCGAGGUGCAGACCGGCGGCUU |
| | | CUCCAAGGAGUCCAUCCUGCCCAAGCGGAACUCCGACAAG |
| | | CUGAUCGCCCGGAAGAAGGACUGGGACCCCAAGAAGUAC |
| | | GGCGGCUUCGACUCCCCCACCGUGGCCUACUCCGUGCUGG |
| | | UGGUGGCCAAGGUGGAGAAGGGCAAGUCCAAGAAGCUGA |
| | | AGUCCGUGAAGGAGCUGCUGGGCAUCACCAUCAUGGAGC |
| | | GGUCCUCCUUCGAGAAGAACCCCAUCGACUUCCUGGAGGC |
| | | CAAGGGCUACAAGGAGGUGAAGAAGGACCUGAUCAUCAA |
| | | GCUGCCCAAGUACUCCCUGUUCGAGCUGGAGAACGGCCG |
| | | GAAGCGGAUGCUGGCCUCCGCCGGCGAGCUGCAGAAGGG |
| | | CAACGAGCUGGCCCUGCCCUCCAAGUACGUGAACUUCCUG |
| | | UACCUGGCCUCCCACUACGAGAAGCUGAAGGGCUCCCCCG |
| | | AGGACAACGAGCAGAAGCAGCUGUUCGUGGAGCAGCACA |
| | | AGCACUACCUGGACGAGAUCAUCGAGCAGAUCUCCGAGU |
| | | UCUCCAAGCGGGUGAUCCUGGCCGACGCCAACCUGGACAA |
| | | GGUGCUGUCCGCCUACAACAAGCACCGGGACAAGCCCAUC |
| | | CGGGAGCAGGCCGAGAACAUCAUCCACCUGUUCACCCUGA |
| | | CCAACCUGGGCGCCCCCGCCGCCUUCAAGUACUUCGACAC |
| | | CACCAUCGACCGGAAGCGGUACACCUCCACCAAGGAGGUG |
| | | CUGGACGCCACCCUGAUCCACCAGUCCAUCACCGGCCUGU |
| | | ACGAGACCCGGAUCGACCUGUCCCAGCUGGGCGGCGACGG |
| | | CGGCGGCUCCCCCAAGAAGAAGCGGAAGGUGUGA |
| RNA encoding | 805 | AUGGACAAGAAGUACAGCAUCGGACUGGACAUCGGAACA |
| Cas9 | | AACAGCGUCGGAUGGGCAGUCAUCACAGACGAAUACAAG |
| corresponding | | GUCCCGAGCAAGAAGUUCAAGGUCCUGGGAAACACAGAC |
| to SEQ ID NO: | | AGACACAGCAUCAAGAAGAACCUGAUCGGAGCACUGCUG |
| 801 | | UUCGACAGCGGAGAAACAGCAGAAGCAACAAGACUGAAG |
| | | AGAACAGCAAGAAGAAGAUACACAAGAAGAAAGAACAGA |
| | | AUCUGCUACCUGCAGGAAAUCUUCAGCAACGAAAUGGCA |
| | | AAGGUCGACGACAGCUUCUUCCACAGACUGGAAGAAAGC |
| | | UUCCUGGUCGAAGAAGACAAGAAGCACGAAAGACACCCG |
| | | AUCUUCGGAAACAUCGUCGACGAAGUCGCAUACCACGAA |
| | | AAGUACCCGACAAUCUACCACCUGAGAAAGAAGCUGGUC |
| | | GACAGCACAGACAAGGCAGACCUGAGACUGAUCUACCUG |
| | | GCACUGGCACACAUGAUCAAGUUCAGAGGACACUUCCUG |
| | | AUCGAAGGAGACCUGAACCCGGACAACAGCGACGUCGAC |
| | | AAGCUGUUCAUCCAGCUGGUCCAGACAUACAACCAGCUG |
| | | UUCGAAGAAAACCCGAUCAACGCAAGCGGAGUCGACGCA |
| | | AAGGCAAUCCUGAGCGCAAGACUGAGCAAGAGCAGAAGA |
| | | CUGGAAAACCUGAUCGCACAGCUGCCGGGAGAAAAGAAG |
| | | AACGGACUGUUCGGAAACCUGAUCGCACUGAGCCUGGGA |
| | | CUGACACCGAACUUCAAGAGCAACUUCGACCUGGCAGAA |
| | | GACGCAAAGCUGCAGCUGAGCAAGGACACAUACGACGAC |
| | | GACCUGGACAACCUGCUGGCACAGAUCGGAGACCAGUAC |
| | | GCAGACCUGUUCCUGGCAGCAAAGAACCUGAGCGACGCA |
| | | AUCCUGCUGAGCGACAUCCUGAGAGUCAACACAGAAAUC |
| | | ACAAAGGCACCGCUGAGCGCAAGCAUGAUCAAGAGAUAC |
| | | GACGAACACCACCAGGACCUGACACUGCUGAAGGCACUG |
| | | GUCAGACAGCAGCUGCCGGAAAAGUACAAGGAAAUCUUC |
| | | UUCGACCAGAGCAAGAACGGAUACGCAGGAUACAUCGAC |
| | | GGAGGAGCAAGCCAGGAAGAAUUCUACAAGUUCAUCAAG |
| | | CCGAUCCUGGAAAAGAUGGACGGAACAGAAGAACUGCUG |
| | | GUCAAGCUGAACAGAGAAGACCUGCUGAGAAAGCAGAGA |
| | | ACAUUCGACAACGGAAGCAUCCCGCACCAGAUCCACCUGG |
| | | GAGAACUGCACGCAAUCCUGAGAAGACAGGAAGACUUCU |
| | | ACCCGUUCCUGAAGGACAACAGAGAAAAGAUCGAAAAGA |
| | | UCCUGACAUUCAGAAUCCCGUACUACGUCGGACCGCUGGC |
| | | AAGAGGAAACAGCAGAUUCGCAUGGAUGACAAGAAAGAG |
| | | CGAAGAAACAAUCACACCGUGGAACUUCGAAGAAGUCGU |
| | | CGACAAGGGAGCAAGCGCACAGAGCUUCAUCGAAAGAAU |
| | | GACAAACUUCGACAAGAACCUGCCGAACGAAAAGGUCCU |
| | | GCCGAAGCACAGCCUGCUGUACGAAUACUUCACAGUCUA |
| | | CAACGAACUGACAAAGGUCAAGUACGUCACAGAAGGAAU |
| | | GAGAAAGCCGGCAUUCCUGAGCGGAGAACAGAAGAAGGC |
| | | AAUCGUCGACCUGCUGUUCAAGACAAACAGAAAGGUCAC |
| | | AGUCAAGCAGCUGAAGGAAGACUACUUCAAGAAGAUCGA |
| | | AUGCUUCGACAGCGUCGAAAUCAGCGGAGUCGAAGACAG |
| | | AUUCAACGCAAGCCUGGGAACAUACCACGACCUGCUGAA |
| | | GAUCAUCAAGGACAAGGACUUCCUGGACAACGAAGAAAA |
| | | CGAAGACAUCCUGGAAGACAUCGUCCUGACACUGACACU |
| | | GUUCGAAGACAGAGAAAUGAUCGAAGAAAGACUGAAGAC |
| | | AUACGCACACCUGUUCGACGACAAGGUCAUGAAGCAGCU |
| | | GAAGAGAAGAAGAUACACAGGAUGGGGAAGACUGAGCAG |
| | | AAAGCUGAUCAACGGAAUCAGAGACAAGCAGAGCGGAAA |
| | | GACAAUCCUGGACUUCCUGAAGAGCGACGGAUUCGCAAA |
| | | CAGAAACUUCAUGCAGCUGAUCCACGACGACAGCCUGAC |
| | | AUUCAAGGAAGACAUCCAGAAGGCACAGGUCAGCGGACA |
| | | GGGAGACAGCCUGCACGAACACAUCGCAAACCUGGCAGG |
| | | AAGCCCGGCAAUCAAGAAGGGAAUCCUGCAGACAGUCAA |
| | | GGUCGUCGACGAACUGGUCAAGGUCAUGGGAAGACACAA |
| | | GCCGGAAAACAUCGUCAUCGAAAUGGCAAGAGAAAACCA |
| | | GACAACACAGAAGGGACAGAAGAACAGCAGAGAAAGAAU |
| | | GAAGAGAAUCGAAGAAGGAAUCAAGGAACUGGGAAGCCA |
| | | GAUCCUGAAGGAACACCCGGUCGAAAACACACAGCUGCA |
| | | GAACGAAAAGCUGUACCUGUACUACCUGCAGAACGGAAG |
| | | AGACAUGUACGUCGACCAGGAACUGGACAUCAACAGACU |
| | | GAGCGACUACGACGUCGACCACAUCGUCCCGCAGAGCUUC |
| | | CUGAAGGACGACAGCAUCGACAACAAGGUCCUGACAAGA |
| | | AGCGACAAGAACAGAGGAAAGAGCGACAACGUCCCGAGC |
| | | GAAGAAGUCGUCAAGAAGAUGAAGAACUACUGGAGACAG |
| | | CUGCUGAACGCAAAGCUGAUCACACAGAGAAAGUUCGAC |
| | | AACCUGACAAAGGCAGAGAGAGGAGGACUGAGCGAACUG |
| | | GACAAGGCAGGAUUCAUCAAGAGACAGCUGGUCGAAACA |
| | | AGACAGAUCACAAAGCACGUCGCACAGAUCCUGGACAGC |
| | | AGAAUGAACACAAAGUACGACGAAAACGACAAGCUGAUC |
| | | AGAGAAGUCAAGGUCAUCACACUGAAGAGCAAGCUGGUC |
| | | AGCGACUUCAGAAAGGACUUCCAGUUCUACAAGGUCAGA |
| | | GAAAUCAACAACUACCACCACGCACACGACGCAUACCUGA |
| | | ACGCAGUCGUCGGAACAGCACUGAUCAAGAAGUACCCGA |
| | | AGCUGGAAAGCGAAUUCGUCUACGGAGACUACAAGGUCU |
| | | ACGACGUCAGAAAGAUGAUCGCAAAGAGCGAACAGGAAA |
| | | UCGGAAAGGCAACAGCAAAGUACUUCUUCUACAGCAACA |
| | | UCAUGAACUUCUUCAAGACAGAAAUCACACUGGCAAACG |
| | | GAGAAAUCAGAAAGAGACCGCUGAUCGAAACAAACGGAG |
| | | AAACAGGAGAAAUCGUCUGGGACAAGGGAAGAGACUUCG |
| | | CAACAGUCAGAAAGGUCCUGAGCAUGCCGCAGGUCAACA |
| | | UCGUCAAGAAGACAGAAGUCCAGACAGGAGGAUUCAGCA |
| | | AGGAAAGCAUCCUGCCGAAGAGAAACAGCGACAAGCUGA |
| | | UCGCAAGAAAGAAGGACUGGGACCCGAAGAAGUACGGAG |
| | | GAUUCGACAGCCCGACAGUCGCAUACAGCGUCCUGGUCG |
| | | UCGCAAAGGUCGAAAAGGGAAAGAGCAAGAAGCUGAAGA |
| | | GCGUCAAGGAACUGCUGGGAAUCACAAUCAUGGAAAGAA |
| | | GCAGCUUCGAAAAGAACCCGAUCGACUUCCUGGAAGCAA |
| | | AGGGAUACAAGGAAGUCAAGAAGGACCUGAUCAUCAAGC |
| | | UGCCGAAGUACAGCCUGUUCGAACUGGAAAACGGAAGAA |
| | | AGAGAAUGCUGGCAAGCGCAGGAGAACUGCAGAAGGGAA |
| | | ACGAACUGGCACUGCCGAGCAAGUACGUCAACUUCCUGU |
| | | ACCUGGCAAGCCACUACGAAAAGCUGAAGGGAAGCCCGG |
| | | AAGACAACGAACAGAAGCAGCUGUUCGUCGAACAGCACA |
| | | AGCACUACCUGGACGAAAUCAUCGAACAGAUCAGCGAAU |
| | | UCAGCAAGAGAGUCAUCCUGGCAGACGCAAACCUGGACA |
| | | AGGUCCUGAGCGCAUACAACAAGCACAGAGACAAGCCGA |
| | | UCAGAGAACAGGCAGAAAACAUCAUCCACCUGUUCACAC |
| | | UGACAAACCUGGGAGCACCGGCAGCAUUCAAGUACUUCG |
| | | ACACAACAAUCGACAGAAAGAGAUACACAAGCACAAAGG |
| | | AAGUCCUGGACGCAACACUGAUCCACCAGAGCAUCACAG |
| | | GACUGUACGAAACAAGAAUCGACCUGAGCCAGCUGGGAG |
| | | GAGACGGAGGAGGAAGCCCGAAGAAGAAGAGAAAGGUCU |
| | | AG |
| HD1 TCR | 1001 | ttggccactccctctctgcgcgctcgctcgctcactgaggccgggcgaccaaaggtcgcccgacg |
| insertion | | cccgggctttgcccgggcggcctcagtgagcgagcgagcgcgcagagagggagtggccaact |
| including ITRs | | ccatcactaggggttcctagatcttgccaacataccataaacctcccattctgctaatgcccagccta |
| | | agttggggagaccactccagattccaagatgtacagtttgctttgctgggcctttttcccatgcctgcc |
| | | tttactctgccagagttatattgctggggttttgaagaagatcctattaaataaaagaataagcagtatt |
| | | attaagtagccctgcatttcaggtttccttgagtggcaggccaggcctggccgtgaacgttcactga |
| | | aatcatggcctcttggccaagattgatagcttgtgcctgtccctgagtcccagtccatcacgagcag |
| | | ctggtttctaagatgctatttcccgtataaagcatgagaccgtgacttgccagccccacagagcccc |
| | | gcccttgtccatcactggcatctggactccagcctgggttggggcaaagagggaaatgagatcatg |
| | | tcctaaccctgatcctcttgtcccacagatatccagaaccctgaccctgcggctccggtgcccgtca |
| | | gtgggcagagcgcacatcgcccacagtccccgagaagttggggggaggggtcggcaattgaac |
| | | cggtgcctagagaaggtggcgcggggtaaactgggaaagtgatgtcgtgtactggctccgcctttt |
| | | tcccgaggggggggagaaccgtatataagtgcagtagtcgccgtgaacgttctttttcgcaacgg |
| | | gtttgccgccagaacacaggtaagtgccgtgtgtggttcccgcgggcctggcctctttacgggttat |
| | | ggcccttgcgtgccttgaattacttccacgcccctggctgcagtacgtgattcttgatcccgagcttc |
| | | gggttggaagtgggtgggagagttcgaggccttgcgcttaaggagccccttcgcctcgtgcttgag |
| | | ttgaggcctggcttgggcgctggggccgccgcgtgcgaatctggtggcaccttcgcgcctgtctc |
| | | gctgctttcgataagtctctagccatttaaaatttttgatgacctgctgcgacgctttttttctggcaag |
| | | atagtcttgtaaatgcgggccaagatgtgcacactggtatttcggtttttggggccgcgggcggcgac |
| | | ggggcccgtgcgtcccagcgcacatgttcggcgaggcggggcctgcgagcgcggccaccgag |
| | | aatcggacgggggtagtctcaagctggccggcctgctctggtgcctggcctcgcgccgccgtgta |
| | | tcgccccgccctgggcggcaaggctggcccggtcggcaccagttgcgtgagcggaaagatggc |
| | | cgcttcccggccctgctgcagggagctcaaaatggaggacgcggcgctcgggagagcgggcg |
| | | ggtgagtcacccacacaaaggaaaagggcctttccgtcctcagccgtcgcttcatgtgactccacg |
| | | gagtaccgggcgccgtccaggcacctcgattagttctcgagcttttggagtacgtcgtctttaggttg |
| | | gggggaggggttttatgcgatggagtttccccacactgagtgggtggagactgaagttaggccag |
| | | cttggcacttgatgtaattctccttggaatttgccctttttgagtttggatcttggttcattctcaagcc |
| | | tcagacagtggttcaaagtttttttcttccatttcaggtgtcgtgatgcggccgccaccatgggatcttg |
| | | gacactgtgttgcgtgtccctgtgcatcctggtggccaagcacacagatgccggcgtgatccagtctcc |
| | | tagacacgaagtgaccgagatgggccaagaagtgaccctgcgctgcaagcctatcagcggccac |
| | | gattacctgttctggtacagacagaccatgatgagaggcctggaactgctgatctacttcaacaaca |
| | | acgtgcccatcgacgacagcggcatgcccgaggatagattcagcgccaagatgcccaacgcca |
| | | gcttcagcaccctgaagatccagcctagcgagcccagagatagcgccgtgtacttctgcgccagc |
| | | agaaagacaggcggctacagcaatcagccccagcactttggagatggcacccggctgagcatcc |
| | | tggaagatctgaagaacgtgttcccacctgaggtggccgtgttcgagccttctgaggccgagatca |
| | | gccacacacagaaagccacactcgtgtgtctggccaccggcttctatcccgatcacgtggaactgt |
| | | cttggtgggtcaacggcaaagaggtgcacagcggcgtcagcaccgatcctcagcctctgaaaga |
| | | gcagcccgctctgaacgacagcagatactgcctgagcagcagactgagagtgtccgccaccttct |
| | | ggcagaaccccagaaaccacttcagatgccaggtgcagttctacggcctgagcgagaacgatga |
| | | gtggacccaggatagagccaagcctgtgacacagatcgtgtctgccgaagcctggggcagagcc |
| | | gattgtggctttaccagcgagagctaccagcagggcgtgctgtctgccacaatcctgtacgagatc |
| | | ctgctgggcaaagccactctgtacgccgtgctggtgtctgccctggtgctgatggccatggtcaag |
| | | cggaaggatagcaggggcggctccggtgccacaaacttctccctgctcaagcaggccggagatg |
| | | tggaagagaaccctggccctatggaaaccctgctgaaggtgctgagcggcacactgctgtggca |
| | | gctgacatgggtccgatctcagcagcctgtgcagtctcctcaggccgtgattctgagagaaggcga |
| | | ggacgccgtgatcaactgcagcagctctaaggccctgtacagcgtgcactggtacagacagaag |
| | | cacggcgaggcccctgtgttcctgatgatcctgctgaaaggcggcgagcagaagggccacgag |
| | | aagatcagcgccagcttcaacgagaagaagcagcagtccagcctgtacctgacagccagccagc |
| | | tgagctacagcggcacctacttttgtggcaccgcctggatcaacgactacaagctgtctttcggagc |
| | | cggcaccacagtgacagtgcgggccaatattcagaaccccgatcctgccgtgtaccagctgaga |
| | | gacagcaagagcagcgacaagagcgtgtgcctgttcaccgacttcgacagccagaccaacgtgt |
| | | cccagagcaaggacagcgacgtgtacatcaccgataagactgtgctggacatgcggagcatgga |
| | | cttcaagagcaacagcgccgtggcctggtccaacaagagcgatttcgcctgcgccaacgccttca |
| | | acaacagcattatccccgaggacacattcttcccaagtcctgagagcagctgcgacgtgaagctg |
| | | gtggaaaagagcttcgagacagacaccaacctgaacttccagaacctgagcgtgatcggcttcag |
| | | aatcctgctgctcaaggtggccggcttcaacctgctgatgaccctgagactgtggtccagctaacct |
| | | CGACTGTGCCTTCTAGTTGCCAGCCATCTGTTGTTTGCCCCT |
| | | CCCCCGTGCCTTCCTTGACCCTGGAAGGTGCCACTCCCACT |
| | | GTCCTTTCCTAATAAAATGAGGAAATTGCATCGCATTGTCT |
| | | GAGTAGGTGTCATTCTATTCTGGGGGGTGGGGTGGGGCAGG |
| | | ACAGCAAGGGGGAGGATTGGGAAGACAATAGCAGGCATGC |
| | | TGGGGATGCGGTGGGCTCTATGGcttctgaggcggaaagaaccagctgggg |
| | | ctctagggggtatccccactagtcgtgtaccagctgagagactctaaatccagtgacaagtctgtct |
| | | gcctattcaccgattttgattctcaaacaaatgtgtcacaaagtaaggattctgatgtgtatatcacaga |
| | | caaaactgtgctagacatgaggtctatggacttcaagagcaacagtgctgtggcctggagcaacaa |
| | | atctgactttgcatgtgcaaacgccttcaacaacagcattattccagaagacaccttcttccccagcc |
| | | caggtaagggcagctttggtgccttcgcaggctgtttccttgcttcaggaatggccaggttctgccc |
| | | agagctctggtcaatgatgtctaaaactcctctgattggtggtctcggccttatccattgccaccaaaa |
| | | ccctctttttactaagaaacagtgagccttgttctggcagtccagagaatgacacgggaaaaaagca |
| | | gatgaagagaaggtggcaggagagggcacgtggcccagcctcagtctctagatctaggaacccc |
| | | tagtgatggagttggccactccctctctgcgcgctcgctcgctcactgaggccgcccgggcaaag |
| | | cccgggcgtcgggcgacctttggtcgcccggcctcagtgagcgagcgagcgcgcagagaggg |
| | | agtggccaa |

### Example 8—Additional Embodiments

The following numbered embodiments provide additional support for and descriptions of the embodiments herein.

Embodiment 1 is an engineered cell comprising a genetic modification in a human PD1 sequence, within genomic coordinates of chr2: 241849881-241858908.

Embodiment 2 is the engineered cell of embodiment, wherein the genetic modification is selected from an insertion, a deletion, and a substitution.

Embodiment 3 is the engineered cell of embodiments 1 or 2, wherein the genetic modification inhibits expression of the PD1 gene.

Embodiment 4 is the engineered cell of any one of embodiments 1-3, wherein the genetic modification comprises a modification of at least one nucleotide within the genomic coordinates selected from:

| PD1 NO. | Genomic Coordinates (hg38) |
|---|---|
| PD1-29 | chr2: 241852703-241852723 |
| PD1-43 | chr2: 241858807-241858827 |
| PD1-5 | chr2: 241858789-241858809 |
| PD1-6 | chr2: 241858788-241858808 |
| PD1-8 | chr2: 241858755-241858775 |
| PD1-11 | chr2: 241852919-241852939 |
| PD1-12 | chr2: 241852915-241852935 |
| PD1-22 | chr2: 241852755-241852775 |
| PD1-23 | chr2: 241852751-241852771 |
| PD1-24 | chr2: 241852750-241852770 |
| PD1-36 | chr2: 241852264-241852284 |
| PD1-57 | chr2: 241852201-241852221 |
| PD1-58 | chr2: 241852749-241852769 |
| PD1-17 | chr2: 241852821-241852841 |
| PD1-38 | chr2: 241852265-241852285 |
| PD1-56 | chr2: 241851221-241851241 |
| PD1-41 | chr2: 241852188-241852208; |

or PD1-11, PD1-12, PD1-24, PD1-36, PD1-38, PD1-43, PD1-57, PD1-5, PD1-6, PD1-8, PD1-22, PD1-23, and PD1-29; or PD1-6, PD1-8, PD1-11, PD1-12, PD1-23, PD1-43, PD1-29; or PD1-5, PD1-11, PD1-12, PD1-22, PD1-23, and PD1-43; or PD1-6, PD1-8, PD1-23, and PD1-29; or PD1-6 and PD1-29; or PD1-6, PD1-23, PD1-29, PD1-41, and PD1-57; or PD1-6, PD1-29, and PD1-57; or PD1-43.

Embodiment 5 is the engineered cell of any one of embodiments 1-4, wherein the engineered cell comprises a genetic modification within the genomic coordinates of an endogenous T cell receptor (TCR) sequence, wherein the genetic modification inhibits expression of the TCR gene.

Embodiment 6 is the engineered cell of embodiment 5, wherein the TCR gene is TRAC or TRBC.

Embodiment 7 is the engineered cell of embodiment 6, comprising a genetic modification of TRBC within genomic coordinates selected from:

| TRBC NO: | Genomic Coordinates (hg38) |
|---|---|
| TRBC-1 | chr7: 142791996-142792016 |
| TRBC-2 | chr7: 142792047-142792067 |
| TRBC-3 | chr7: 142792008-142792028 |
| TRBC-4 | chr7: 142791931-142791951 |
| TRBC-5 | chr7: 142791930-142791950 |
| TRBC-6 | chr7: 142791748-142791768 |
| TRBC-7 | chr7: 142791720-142791740 |
| TRBC-8 | chr7: 142792041-142792061 |
| TRBC-9 | chr7: 142802114-142802134 |
| TRBC-10 | chr7: 142792009-142792029 |
| TRBC-11 | chr7: 142792697-142792717 |
| TRBC-12 | chr7: 142791963-142791983 |
| TRBC-13 | chr7: 142791976-142791996 |
| TRBC-14 | chr7: 142791974-142791994 |
| TRBC-15 | chr7: 142791970-142791990 |
| TRBC-16 | chr7: 142791948-142791968 |
| TRBC-17 | chr7: 142791913-142791933 |
| TRBC-18 | chr7: 142791961-142791981 |
| TRBC-19 | chr7: 142792068-142792088 |
| TRBC-20 | chr7: 142791975-142791995 |
| TRBC-21 | chr7: 142791773-142791793 |
| TRBC-22 | chr7: 142791919-142791939 |
| TRBC-23 | chr7: 142791834-142791854 |
| TRBC-24 | chr7: 142791878-142791898 |
| TRBC-25 | chr7: 142802141-142802161 |
| TRBC-26 | chr7: 142791844-142791864 |
| TRBC-27 | chr7: 142801154-142801174 |
| TRBC-28 | chr7: 142791961-142791981 |
| TRBC-29 | chr7: 142792001-142792021 |
| TRBC-30 | chr7: 142791979-142791999 |
| TRBC-31 | chr7: 142792041-142792061 |
| TRBC-32 | chr7: 142792003-142792023 |
| TRBC-33 | chr7: 142791984-142792004 |
| TRBC-34 | chr7: 142792002-142792022 |
| TRBC-35 | chr7: 142791966-142791986 |
| TRBC-36 | chr7: 142792007-142792027 |
| TRBC-37 | chr7: 142791993-142792013 |
| TRBC-38 | chr7: 142791902-142791922 |
| TRBC-39 | chr7: 142791724-142791744 |
| TRBC-40 | chr7: 142791973-142791993 |
| TRBC-41 | chr7: 142791920-142791940 |
| TRBC-42 | chr7: 142791994-142792014 |
| TRBC-43 | chr7: 142791887-142791907 |
| TRBC-44 | chr7: 142791907-142791927 |
| TRBC-45 | chr7: 142791952-142791972 |
| TRBC-46 | chr7: 142791721-142791741 |
| TRBC-47 | chr7: 142792718-142792738 |
| TRBC-48 | chr7: 142791729-142791749 |
| TRBC-49 | chr7: 142791911-142791931 |
| TRBC-50 | chr7: 142791867-142791887 |
| TRBC-51 | chr7: 142791899-142791919 |
| TRBC-52 | chr7: 142791727-142791747 |
| TRBC-53 | chr7: 142791949-142791969 |
| TRBC-54 | chr7: 142791933-142791953 |
| TRBC-55 | chr7: 142791932-142791952 |
| TRBC-56 | chr7: 142792057-142792077 |
| TRBC-57 | chr7: 142791940-142791960 |
| TRBC-58 | chr7: 142791747-142791767 |
| TRBC-59 | chr7: 142791881-142791901 |
| TRBC-60 | chr7: 142791779-142791799 |
| TRBC-61 | chr7: 142792054-142792074 |
| TRBC-62 | chr7: 142792069-142792089 |
| TRBC-63 | chr7: 142792712-142792732 |
| TRBC-64 | chr7: 142791729-142791749 |
| TRBC-65 | chr7: 142791821-142791841 |
| TRBC-66 | chr7: 142792052-142792072 |
| TRBC-67 | chr7: 142791916-142791936 |
| TRBC-68 | chr7: 142791899-142791919 |
| TRBC-69 | chr7: 142791772-142791792 |
| TRBC-70 | chr7: 142792714-142792734 |
| TRBC-71 | chr7: 142792042-142792062 |
| TRBC-72 | chr7: 142791962-142791982 |
| TRBC-73 | chr7: 142791988-142792008 |
| TRBC-74 | chr7: 142791982-142792002 |
| TRBC-75 | chr7: 142792049-142792069 |
| TRBC-76 | chr7: 142791839-142791859 |
| TRBC-77 | chr7: 142791893-142791913 |
| TRBC-78 | chr7: 142791945-142791965 |
| TRBC-79 | chr7: 142791964-142791984 |
| TRBC-80 | chr7: 142791757-142791777 |
| TRBC-81 | chr7: 142792048-142792068 |
| TRBC-82 | chr7: 142791774-142791794 |
| TRBC-83 | chr7: 142792048-142792068 |
| TRBC-84 | chr7: 142791830-142791850 |
| TRBC-85 | chr7: 142791909-142791929 |
| TRBC-86 | chr7: 142791912-142791932 |
| TRBC-87 | chr7: 142791766-142791786 |
| TRBC-88 | chr7: 142791880-142791900 |
| TRBC-89 | chr7: 142791919-142791939 |

Embodiment 8 is the engineered cell of any one of embodiments 5-7, comprising a genetic modification of TRAC within genomic coordinates selected from:

| TRAC NO: | Genomic Coordinates (hg38) |
|---|---|
| TRAC-90 | chr14: 22547524-22547544 |
| TRAC-91 | chr14: 22550581-22550601 |
| TRAC-92 | chr14: 22550608-22550628 |
| TRAC-93 | chr14: 22550611-22550631 |
| TRAC-94 | chr14: 22550622-22550642 |
| TRAC-95 | chr14: 22547529-22547549 |
| TRAC-96 | chr14: 22547512-22547532 |
| TRAC-97 | chr14: 22547525-22547545 |
| TRAC-98 | chr14: 22547536-22547556 |
| TRAC-99 | chr14: 22547575-22547595 |
| TRAC-100 | chr14: 22547640-22547660 |
| TRAC-101 | chr14: 22547647-22547667 |
| TRAC-102 | chr14: 22547777-22547797 |
| TRAC-103 | chr14: 22549638-22549658 |
| TRAC-104 | chr14: 22549646-22549666 |
| TRAC-105 | chr14: 22550600-22550620 |
| TRAC-106 | chr14: 22550605-22550625 |
| TRAC-107 | chr14: 22550625-22550645 |
| TRAC-108 | chr14: 22539116-22539136 |
| TRAC-109 | chr14: 22539120-22539140 |
| TRAC-110 | chr14: 22547518-22547538 |
| TRAC-111 | chr14: 22539082-22539102 |
| TRAC-112 | chr14: 22539061-22539081 |
| TRAC-113 | chr14: 22539097-22539117 |
| TRAC-114 | chr14: 22547697-22547717 |
| TRAC-115 | chr14: 22550571-22550591 |
| TRAC-116 | chr14: 22550631-22550651 |
| TRAC-117 | chr14: 22550658-22550678 |
| TRAC-118 | chr14: 22547712-22547732 |
| TRAC-119 | chr14: 22550636-22550656 |
| TRAC-120 | chr14: 22550636-22550656 |
| TRAC-121 | chr14: 22550582-22550602 |
| TRAC-122 | chr14: 22550606-22550626 |
| TRAC-123 | chr14: 22550609-22550629 |
| TRAC-124 | chr14: 22547691-22547711 |
| TRAC-125 | chr14: 22547576-22547596 |
| TRAC-126 | chr14: 22549648-22549668 |
| TRAC-127 | chr14: 22549660-22549680 |
| TRAC-128 | chr14: 22547716-22547736 |
| TRAC-129 | chr14: 22547514-22547534 |
| TRAC-130 | chr14: 22550662-22550682 |
| TRAC-131 | chr14: 22550593-22550613 |
| TRAC-132 | chr14: 22550612-22550632 |
| TRAC-133 | chr14: 22547521-22547541 |
| TRAC-134 | chr14: 22547540-22547560 |
| TRAC-135 | chr14: 22539121-22539141 |
| TRAC-136 | chr14: 22547632-22547652 |
| TRAC-137 | chr14: 22547674-22547694 |
| TRAC-138 | chr14: 22549643-22549663 |
| TRAC-139 | chr14: 22547655-22547675 |
| TRAC-140 | chr14: 22547667-22547687 |
| TRAC-141 | chr14: 22539085-22539105 |
| TRAC-142 | chr14: 22549634-22549654 |
| TRAC-143 | chr14: 22539064-22539084 |
| TRAC-144 | chr14: 22547639-22547659 |
| TRAC-145 | chr14: 22547731-22547751 |
| TRAC-146 | chr14: 22547734-22547754 |
| TRAC-147 | chr14: 22547591-22547611 |
| TRAC-148 | chr14: 22547657-22547677 |
| TRAC-149 | chr14: 22547519-22547539 |
| TRAC-150 | chr14: 22549674-22549694 |
| TRAC-151 | chr14: 22547678-22547698 |
| TRAC-152 | chr14: 22539087-22539107 |
| TRAC-153 | chr14: 22547595-22547615 |
| TRAC-154 | chr14: 22547633-22547653 |
| TRAC-155 | chr14: 22547732-22547752 |
| TRAC-156 | chr14: 22547656-22547676 |
| TRAC-157 | chr14: 22539086-22539106 |
| TRAC-158 | chr14: 22547491-22547511 |
| TRAC-159 | chr14: 22547618-22547638 |
| TRAC-160 | chr14: 22549644-22549664 |
| TRAC-161 | chr14: 22547522-22547542 |
| TRAC-162 | chr14: 22539089-22539109 |
| TRAC-163 | chr14: 22539062-22539082 |
| TRAC-164 | chr14: 22547597-22547617 |
| TRAC-165 | chr14: 22547677-22547697 |
| TRAC-166 | chr14: 22549645-22549665 |
| TRAC-167 | chr14: 22550610-22550630 |
| TRAC-168 | chr14: 22547511-22547531 |
| TRAC-169 | chr14: 22550607-22550627 |
| TRAC-170 | chr14: 22550657-22550677 |
| TRAC-171 | chr14: 22550604-22550624 |
| TRAC-172 | chr14: 22539132-22539152 |
| TRAC-173 | chr14: 22550632-22550652 |
| TRAC-174 | chr14: 22547571-22547591 |
| TRAC-175 | chr14: 22547711-22547731 |
| TRAC-176 | chr14: 22547666-22547686 |
| TRAC-177 | chr14: 22547567-22547587 |
| TRAC-178 | chr14: 22547624-22547644 |
| TRAC-185 | chr14: 22547501-22547521 |
| TRAC-213 | chr14: 22547519-22547539 |
| TRAC-214 | chr14: 22547556-22547576 |
| TRAC-215 | chr14: 22547486-22547506 |
| TRAC-216 | chr14: 22547487-22547507 |
| TRAC-217 | chr14: 22547493-22547513 |
| TRAC-218 | chr14: 22547502-22547522 |

optionally wherein the genetic modification is within genomic coordinates selected from chr14:22547524-22547544, chr14:22547529-22547549, chr14:22547525-22547545, chr14:22547536-22547556, chr14:22547501-22547521, chr14:22547556-22547576, and chr14:22547502-22547522.

Embodiment 9 is the engineered cell of any one of embodiments 1-8, wherein the cell comprises a genetic modification, wherein the genetic modification inhibits expression of one or more MHC class I proteins.

Embodiment 10 is the engineered cell of embodiment 9, wherein the genetic modification that inhibits expression of one or more MHC class I proteins is a genetic modification in a B2M sequence, wherein the genetic modification is within genomic coordinates selected from:

| | | SEQ | |
|---|---|---|---|
| Genomic Location | | ID | |
| (hg38) | Guide Sequence | NO | B2M-# |
| chr15:44711469- | UGGCUGGGCA | 116 | 1 |
| 44711494 | CGCGUUUAAU AUAAG | | |
| chr15:44711472- | CUGGGCACGC | 117 | 2 |
| 44711497 | GUUUAAUAUA AGUGG | | |
| chr15:44711483- | UUUAAUAUAA | 118 | 3 |
| 44711508 | GUGGAGGCGU CGCGC | | |
| chr15:44711486- | AAUAUAAGUG | 119 | 4 |
| 44711511 | GAGGCGUCGC GCUGG | | |
| chr15:44711487- | AUAUAAGUGG | 120 | 5 |
| 44711512 | AGGCGUCGCG CUGGC | | |
| chr15:44711512- | GGGCAUUCCU | 121 | 6 |
| 44711537 | GAAGCUGACA GCAUU | | |
| chr15:44711513- | GGCAUUCCUG | 122 | 7 |
| 44711538 | AAGCUGACAG CAUUC | | |
| chr15:44711534- | AUUCGGGCCG | 123 | 8 |
| 44711559 | AGAUGUCUCG CUCCG | | |
| chr15:44711568- | CUGUGCUCGC | 124 | 9 |
| 44711593 | GCUACUCUCU CUUUC | | |
| chr15:44711573- | CUCGCGCUAC | 125 | 10 |
| 44711598 | UCUCUCUUUC UGGCC | | |
| chr15:44711576- | GCGCUACUCU | 126 | 11 |
| 44711601 | CUCUUUCUGG CCUGG | | |
| chr15:44711466- | AUAUUAAACG | 127 | 12 |
| 44711491 | CGUGCCCAGC CAAUC | | |
| chr15:44711522- | UCUCGGCCCG | 128 | 13 |
| 44711547 | AAUGCUGUCA GCUUC | | |
| chr15:44711544- | GCUAAGGCCA | 129 | 14 |
| 44711569 | CGGAGCGAGA CAUCU | | |
| chr15:44711559- | AGUAGCGCGA | 130 | 15 |
| 44711584 | GCACAGCUAA GGCCA | | |
| chr15:44711565- | AGAGAGAGUA | 131 | 16 |
| 44711590 | GCGCGAGCAC AGCUA | | |
| chr15:44711599- | GAGAGACUCA | 132 | 17 |
| 44711624 | CGCUGGAUAG CCUCC | | |
| chr15:44711611- | GCGGGAGGGU | 133 | 18 |
| 44711636 | AGGAGAGACU CACGC | | |
| chr15:44715412- | UAUUCCUCAG | 134 | 19 |
| 44715437 | GUACUCCAAA GAUUC | | |
| chr15:44715440- | UUUACUCACG | 135 | 20 |
| 44715465 | UCAUCCAGCA GAGAA | | |
| chr15:44715473- | CAAAUUUCCU | 136 | 21 |
| 44715498 | GAAUUGCUAU GUGUC | | |
| chr15:44715474- | AAAUUUCCUG | 137 | 22 |
| 44715499 | AAUUGCUAUG UGUCU | | |
| chr15:44715515- | ACAUUGAAGU | 138 | 23 |
| 44715540 | UGACUUACUG AAGAA | | |
| chr15:44715535- | AAGAAUGGAG | 139 | 24 |
| 44715560 | AGAGAAUUGA AAAAG | | |
| chr15:44715562- | GAGCAUUCAG | 140 | 25 |
| 44715587 | ACUUGUCUUU CAGCA | | |
| chr15:44715567- | UUCAGACUUG | 141 | 26 |
| 44715592 | UCUUUCAGCA AGGAC | | |
| chr15:44715672- | UUUGUCACAG | 142 | 27 |
| 44715697 | CCCAAGAUAG UUAAG | | |
| chr15:44715673- | UUGUCACAGC | 143 | 28 |
| 44715698 | CCAAGAUAGU UAAGU | | |
| chr15:44715674- | UGUCACAGCC | 144 | 29 |
| 44715699 | CAAGAUAGUU AAGUG | | |
| chr15:44715410- | AUCUUUGGAG | 145 | 30 |
| 44715435 | UACCUGAGGA AUAUC | | |
| chr15:44715411- | AAUCUUUGGA | 146 | 31 |
| 44715436 | GUACCUGAGG AAUAU | | |
| chr15:44715419- | UAAACCUGAA | 147 | 32 |
| 44715444 | UCUUUGGAGU ACCUG | | |
| chr15:44715430- | GAUGACGUGA | 148 | 33 |
| 44715455 | GUAAACCUGA AUCUU | | |
| chr15:44715457- | GGAAAUUUGA | 149 | 34 |
| 44715482 | CUUUCCAUUC UCUGC | | |
| chr15:44715483- | AUGAAACCCA | 150 | 35 |
| 44715508 | GACACAUAGC AAUUC | | |
| chr15:44715511- | UCAGUAAGUC | 151 | 36 |
| 44715536 | AACUUCAAUG UCGGA | | |
| chr15:44715515- | UUCUUCAGUA | 152 | 37 |
| 44715540 | AGUCAACUUC AAUGU | | |
| chr15:44715629- | CAGGCAUACU | 153 | 38 |
| 44715654 | CAUCUUUUUC AGUGG | | |
| chr15:44715630- | GCAGGCAUAC | 154 | 39 |
| 44715655 | UCAUCUUUUU CAGUG | | |
| chr15:44715631- | GGCAGGCAUA | 155 | 40 |
| 44715656 | CUCAUCUUUU UCAGU | | |
| chr15:4471S632- | CGGCAGGCAU | 156 | 41 |
| 44715657 | ACUCAUCUUU UUCAG | | |
| chr15:44715653- | GACAAAGUCA | 157 | 42 |
| 44715678 | CAUGGUUCAC ACGGC | | |
| chr15:44715657- | CUGUGACAAA | 158 | 43 |
| 44715682 | GUCACAUGGU UCACA | | |
| chr15:44715666- | UAUCUUGGGC | 159 | 44 |
| 44715691 | UGUGACAAAG UCACA | | |
| chr15:44715685- | AAGACUUACC | 160 | 45 |
| 44715710 | CCACUUAACU AUCUU | | |
| chr15:44715686- | UAAGACUUAC | 161 | 46 |
| 44715711 | CCCACUUAAC UAUCU | | |
| chr15:44716326- | AGAUCGAGAC | 162 | 47 |
| 44716351 | AUGUAAGCAG CAUCA | | |
| chr15:44716329- | UCGAGACAUG | 163 | 48 |
| 44716354 | UAAGCAGCAU CAUGG | | |
| chr15:44716313- | AUGUCUCGAU | 164 | 49 |
| 44716338 | CUAUGAAAAA GACAG | | |
| chr15:44717599- | UUUUCAGGUU | 165 | 50 |
| 44717624 | UGAAGAUGCC GCAUU | | |
| chr15:44717604- | AGGUUUGAAG | 166 | 51 |
| 44717629 | AUGCCGCAUU UGGAU | | |
| chr15:44717681- | CACUUACACU | 167 | 52 |
| 44717706 | UUAUGCACAA AAUGU | | |
| chr15:44717682- | ACUUACACUU | 168 | 53 |
| 44717707 | UAUGCACAAA AUGUA | | |
| chr15:44717702- | AUGUAGGGUU | 169 | 54 |
| 44717727 | AUAAUAAUGU UAACA | | |
| chr15:44717764- | GUCUCCAUGU | 170 | 55 |
| 44717789 | UUGAUGUAUC UGAGC | | |
| chr15:44717776- | GAUGUAUCUG | 171 | 56 |
| 44717801 | AGCAGGUUGC UCCAC | | |
| chr15:44717786- | AGCAGGUUGC | 172 | 57 |
| 44717811 | UCCACAGGUA GCUCU | | |
| chr15:44717789- | AGGUUGCUCC | 173 | 58 |
| 44717814 | ACAGGUAGCU CUAGG | | |
| chr15:44717790- | GGUUGCUCCA | 174 | 59 |
| 44717815 | CAGGUAGCUC UAGGA | | |
| chr15:44717794- | GCUCCACAGG | 175 | 60 |
| 44717819 | UAGCUCUAGG AGGGC | | |
| chr15:44717805- | AGCUCUAGGA | 176 | 61 |
| 44717830 | GGGCUGGCAA CUUAG | | |
| chr15:44717808- | UCUAGGAGGG | 177 | 62 |
| 44717833 | CUGGCAACUU AGAGG | | |
| chr15:44717809- | CUAGGAGGGC | 178 | 63 |
| 44717834 | UGGCAACUUA GAGGU | | |
| chr15:44717810- | UAGGAGGGCU | 179 | 64 |
| 44717835 | GGCAACUUAG AGGUG | | |
| chr15:44717846- | AUUCUCUUAU | 180 | 65 |
| 44717871 | CCAACAUCAA CAUCU | | |
| chr15:44717945- | CAAUUUACAU | 181 | 66 |
| 44717970 | ACUCUGCUUA GAAUU | | |
| chr15:44717946- | AAUUUACAUA | 182 | 67 |
| 44717971 | CUCUGCUUAG AAUUU | | |
| chr15:44717947- | AUUUACAUAC | 183 | 68 |
| 44717972 | UCUGCUUAGA AUUUG | | |
| chr15:44717948- | UUUACAUACU | 184 | 69 |
| 44717973 | CUGCUUAGAA UUUGG | | |
| chr15:44717973- | GGGAAAAUUU | 185 | 70 |
| 44717998 | AGAAAUAUAA UUGAC | | |
| chr15:44717981- | UUAGAAAUAU | 186 | 71 |
| 44718006 | AAUUGACAGG AUUAU | | |
| chr15:44718056- | UACUUCUUAU | 187 | 72 |
| 44718081 | ACAUUUGAUA AAGUA | | |
| chr15:44718061- | CUUAUACAUU | 188 | 73 |
| 44718086 | UGAUAAAGUA AGGCA | | |
| chr15:44718067- | CAUUUGAUAA | 189 | 74 |
| 44718092 | AGUAAGGCAU GGUUG | | |
| chr15:44718076- | AAGUAAGGCA | 190 | 75 |
| 44718101 | UGGUUGUGGU UAAUC | | |
| chr15:44717589- | CUUCAAACCU | 191 | 76 |
| 44717614 | GAAAAGAAAA GAAAA | | |
| chr15:44717620- | AUUUGGAAUU | 192 | 77 |
| 44717645 | CAUCCAAUCC AAAUG | | |
| chr15:44717642- | UAUUAAAAAG | 193 | 78 |
| 44717667 | CAAGCAAGCA GAAUU | | |
| chr15:44717771- | GCAACCUGCU | 194 | 79 |
| 44717796 | CAGAUACAUC AAACA | | |
| chr15:44717800- | UUGCCAGCCC | 195 | 80 |
| 44717825 | UCCUAGAGCU ACCUG | | |
| chr15:44717859- | UCAAAUCUGA | 196 | 81 |
| 44717884 | CCAAGAUGUU GAUGU | | |
| chr15:44717947- | CAAAUUCUAA | 197 | 82 |
| 44717972 | GCAGAGUAUG UAAAU | | |
| chr15:44718119- | CAAGUUUUAU | 198 | 83 |
| 44718144 | GAUUUAUUUA ACUUG | | |

Embodiment 11 is the engineered cell of embodiment 9, wherein the genetic modification that inhibits expression of one or more MHC class I proteins is a genetic modification in an HLA-A sequence and optionally wherein the genetic modification is within genomic coordinates chosen from chr6:29942854 to chr6:29942913 and chr6:29943518 to chr6: 29943619, optionally genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 12 is the engineered cell of any one of the previous embodiments, wherein the cell comprises a genetic modification, wherein the genetic modification inhibits expression of one or more MHC class II proteins.

Embodiment 13 is the engineered cell of embodiment 12, wherein the genetic modification that inhibits expression of one or more MHC class II proteins is a genetic modification in a CIITA sequence, wherein the genetic modification is within the genomic coordinates selected from chr: 16:10902171-10923242, optionally, chr16: 10902662-10923285, chr16:10906542-10923285, or chr16:10906542-10908121, optionally chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150; optionally chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10922153-10922173, chr16:10916362-10916382, chr16:10916450-10916470, chr16:10923222-10923242, chr16:10910176-10910196, chr16:10895742-10895762, chr16:10916449-10916469, chr16:10923214-10923234, chr16:10906492-10906512, and chr16:10906487-1090650; or optionally chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

Embodiment 14 is the engineered cell of embodiments 12 or 13, wherein the genetic modification that inhibits expression of one or more MHC class II proteins a modification of at least one nucleotide of a CIITA splice site, optionally
a) a modification of at least one nucleotide of a CIITA splice donor site; and/or
b) a modification of a CIITA splice site boundary nucleotide.

Embodiment 15 is the engineered cell of any one of embodiments 1-14, wherein the cell has reduced cell surface expression of PD1 protein.

Embodiment 16 is the engineered cell of any one of embodiments 1-15, wherein the cell has reduced cell surface expression of PD1 protein and reduced cell surface expression of TRAC protein.

Embodiment 17 is the engineered cell of embodiments 15 or 16 further comprising reduced cell surface expression of a TRBC protein.

Embodiment 18 is the engineered cell of any one of embodiments 15-17, wherein cell surface expression of PD1 is below the level of detection.

Embodiment 19 is the engineered cell of any one of embodiments 15-18, wherein cell surface expression of at least one of TRAC and TRBC is below the level of detection.

Embodiment 20 is the engineered cell of embodiment 19, wherein cell surface expression of each of PD1, TRAC, and TRBC is below the level of detection.

Embodiment 21 is the engineered cell of any one of the previous embodiments, comprising a genetic modification in a human 2B4/CD244 sequence, within genomic coordinates of chr1:5016-37743.

Embodiment 22 is the engineered cell of embodiment 21, wherein the genetic modification in 2B4/CD244 is within genomic coordinates selected from:

| 2B4 NO | Genomic Coordinates (hg38) |
|---|---|
| 2B4-1 | chr1: 160841611-160841631 |
| 2B4-2 | chr1: 160841865-160841885 |
| 2B4-3 | chr1: 160862624-160862644 |
| 2B4-4 | chr1: 160862671-160862691 |
| 2B4-5 | chr1: 160841622-160841642 |
| 2B4-6 | chr1: 160841819-160841839 |
| 2B4-7 | chr1: 160841823-160841843 |
| 2B4-8 | chr1: 160841717-160841737 |
| 2B4-9 | chr1: 160841859-160841879 |
| 2B4-10 | chr1: 160841806-160841826 |
| 2B4-11 | chr1: 160841834-160841854 |
| 2B4-12 | chr1: 160841780-160841800 |
| 2B4-13 | chr1: 160841713-160841733 |
| 2B4-14 | chr1: 160841631-160841651 |
| 2B4-15 | chr1: 160841704-160841724 |
| 2B4-16 | chr1: 160841584-160841604 |
| 2B4-17 | chr1: 160841679-160841699 |
| 2B4-18 | chr1: 160841874-160841894 |
| 2B4-19 | chr1: 160841750-160841770 |
| 2B4-20 | chr1: 160841577-160841597 |
| 2B4-21 | chr1: 160841459-160841479 |
| 2B4-22 | chr1: 160841466-160841486 |
| 2B4-23 | chr1: 160841461-160841481 |
| 2B4-24 | chr1: 160841460-160841480 |
| 2B4-25 | chr1: 160841360-160841380 |
| 2B4-26 | chr1: 160841304-160841324 |
| 2B4-27 | chr1: 160841195-160841215 |
| 2B4-28 | chr1: 160841305-160841325 |

optionally the genomic coordinates selected from 2B4-1 through 2B4-5; 2B4-1 and 2B4-2; or 2B4-3, 2B4-4, 2B4-10, and 2B4-17.

Embodiment 23 is the engineered cell of any one of the previous embodiments, comprising a genetic modification in a human TIM3 sequence, within the genomic coordinates of chr5:157085832-157109044.

Embodiment 24 is the engineered cell of embodiment 23, wherein the genetic modification in TIM3 is within genomic coordinates selected from:

| TIM 3 NO | Genomic Coordinates (hg38) |
|---|---|
| TIM3 - 1 | chr5: 157106867-157106887 |
| TIM3 - 2 | chr5: 157106862-157106882 |
| TIM3 - 3 | chr5: 157106803-157106823 |
| TIM3 - 4 | chr5: 157106850-157106870 |
| TIM3 - 5 | chr5: 157104726-157104746 |
| TIM3 - 6 | chr5: 157106668-157106688 |
| TIM3 - 7 | chr5: 157104681-157104701 |
| TIM3 - 8 | chr5: 157104681-157104701 |
| TIM3 - 9 | chr5: 157104680-157104700 |
| TIM3 - 10 | chr5: 157106676-157106696 |
| TIM3 - 11 | chr5: 157087271-157087291 |
| TIM3 - 12 | chr5: 157095432-157095452 |
| TIM3 - 13 | chr5: 157095361-157095381 |
| TIM3 - 14 | chr5: 157095360-157095380 |
| TIM3 - 15 | chr5: 157108945-157108965 |
| TIM3 - 18 | chr5: 157106751-157106771 |
| TIM3 - 19 | chr5: 157095419-157095439 |
| TIM3 - 22 | chr5: 157104679-157104699 |
| TIM3 - 23 | chr5: 157106824-157106844 |
| TIM3 - 26 | chr5: 157087117-157087137 |
| TIM3 - 29 | chr5: 157095379-157095399 |
| TIM3 - 32 | chr5: 157106864-157106884 |
| TIM3 - 42 | chr5: 157095405-157095425 |
| TIM3 - 44 | chr5: 157095404-157095424 |
| TIM3 - 56 | chr5: 157106888-157106908 |
| TIM3 - 58 | chr5: 157087126-157087146 |
| TIM3 - 59 | chr5: 157087253-157087273 |
| TIM3 - 62 | chr5: 157106889-157106909 |
| TIM3 - 63 | chr5: 157106935-157106955 |
| TIM3 - 66 | chr5: 157106641-157106661 |
| TIM3 - 69 | chr5: 157087084-157087104 |
| TIM3 - 75 | chr5: 157104663-157104683 |
| TIM3 - 82 | chr5: 157106875-157106895 |
| TIM3 - 86 | chr5: 157087184-157087204 |
| TIM3 - 87 | chr5: 157106936-157106956 |
| TIM3 - 88 | chr5: 157104696-157104716 |

optionally wherein the genomic coordinates selected from TIM3-1 through TIM3-4, TIM3-6 through TIM3-15, TIM3-18, TIM3-19, TIM3-22, TIM3-29, TIM3-42, TIM3-44, TIM3-58, TIM3-62, TIM3-69, TIM3-82, TIM3-86, and TIM3-88; TIM3-1 through TIM3-5, TIM3-7, TIM3-8, TIM3-12 through TIM3-15, TIM3-23, TIM3-26, TIM3-32, TIM3-56, TIM3-59, TIM3-63, TIM3-66, TIM3-75, and TIM3-87; TIM3-2, TIM3-4, TIM3-15, TIM3-23, TIM3-56, TIM3-59, TIM3-63, TIM3-75, and TIM3-87; TIM3-1 through TIM3-4; TIM3-2, TIM-4, and TIM3-15; TIM3-2, TIM-4, TIM3-15, TIM3-63, and TIM3-87; TIM3-2 and TIM3-15; TIM3-63 and TIM3-87; or TIM3-15.

Embodiment 25 is the engineered cell of any one of the previous embodiments, comprising a genetic modification in a human LAG3 sequence, within the genomic coordinates of chr12: 6772483-6778455.

Embodiment 26 is the engineered cell of embodiment 25, wherein the genetic modification is within the genetic coordinates selected from:

| LAG 3 NO | Genomic Coordinates (hg38) |
|---|---|
| LAG3-1 | chr12: 6773938-6773958 |
| LAG3-2 | chr12: 6774678-6774698 |
| LAG3-3 | chr12: 6772894-6772914 |
| LAG3-4 | chr12: 6774816-6774836 |
| LAG3-5 | chr12: 6774742-6774762 |
| LAG3-6 | chr12: 6775380-6775400 |
| LAG3-7 | chr12: 6774727-6774747 |
| LAG3-8 | chr12: 6774732-6774752 |
| LAG3- 9 | chr12: 6777435-6777455 |
| LAG3- 10 | chr12: 6774771-6774791 |
| LAG3- 11 | chr12: 6772909-6772929 |
| LAG3- 12 | chr12: 6774735-6774755 |
| LAG3- 13 | chr12: 6773783-6773803 |
| LAG3- 14 | chr12: 6775292-6775312 |
| LAG3- 15 | chr12: 6777433-6777453 |
| LAG3- 16 | chr12: 6778268-6778288 |
| LAG3- 17 | chr12: 6775444-6775464 |
| LAG3-24 | chr12: 6777783-6777803 |
| LAG3-26 | chr12: 6777784-6777804 |
| LAG3-41 | chr12: 6778252-6778272 |
| LAG3-59 | chr12: 6777325-6777345 |
| LAG3-83 | chr12: 6777329-6777349 |

optionally wherein the genomic coordinates selected from LAG3-1 through LAG3-15: LAG3-1 through LAG3-11: LAG3-1 through LAG3-4; or LAG3-1, LAG3-4, LAG3-5, and LAG3-9.

Embodiment 27 is the engineered cell of any one of embodiments 21-26, wherein the genetic modification in the indicated genomic coordinates is selected from an insertion, a deletion, and a substitution.

Embodiment 28 is the engineered cell of any one of embodiments 21-27, wherein the genetic modification inhibits expression of the gene in which the genetic modification is present.

Embodiment 29 is the engineered cell of any one of the previous embodiments, wherein the genetic modification comprises an indel.

Embodiment 30 is the engineered cell of any one the previous embodiments, wherein the genetic modification comprises an insertion of a heterologous coding sequence.

Embodiment 31 is the engineered cell of any one the previous embodiments, wherein the genetic modification comprises a substitution.

Embodiment 32 is the engineered cell of embodiment 31, wherein the substitution comprises a C to T substitution or an A to G substitution.

Embodiment 33 is the engineered cell of any one of the previous embodiments, wherein the genetic modification results in a change in the nucleic acid sequence that prevents translation of a full-length protein having an amino acid sequence of the full-length protein prior to genetic modification.

Embodiment 34 is the engineered cell of embodiment 33, wherein the genetic modification results in a change in the nucleic acid sequence that results in a premature stop codon in a coding sequence of the full-length protein.

Embodiment 35 is the engineered cell of embodiment 34, wherein the genetic modification results in a change in the nucleic acid sequence that results in a change in splicing of a pre-mRNA from the genomic locus.

Embodiment 36 is the engineered cell of any one of the previous embodiments, wherein the inhibition results in reduced cell surface expression of a protein from the gene comprising a genetic modification.

Embodiment 37 is the engineered cell of any one of the previous embodiments, wherein the inhibition results in reduced cell surface expression of a protein regulated by the gene comprising a genetic modification.

Embodiment 38 is the engineered cell of any one of the previous embodiments, wherein the cell comprises an exogenous nucleic acid encoding a targeting receptor that is expressed on the surface of the engineered cell.

Embodiment 39 is the engineered cell of embodiment 38, wherein the targeting receptor is a CAR.

Embodiment 40 is the engineered cell of embodiment 38, wherein the targeting receptor is a TCR.

Embodiment 41 is the engineered cell of embodiment 40, wherein the targeting receptor is a WT1 TCR.

Embodiment 42 is the engineered cell of any one of the previous embodiments, wherein the engineered cell is an immune cell.

Embodiment 43 is the engineered cell of embodiment 42, wherein the engineered cell is a monocyte, macrophage, mast cell, dendritic cell, or granulocyte.

Embodiment 44 is the engineered cell of embodiment 43, wherein the engineered cell is a lymphocyte.

Embodiment 45 is the engineered cell of embodiment 44, wherein the engineered cell is a T cell.

Embodiment 46 is a pharmaceutical composition comprising the engineered cell of any one of embodiments 1-45.

Embodiment 47 is a population of cells comprising the engineered cell of any one of embodiments 1-45.

Embodiment 48 is a pharmaceutical composition comprising a population of cells, wherein the population of cells comprises engineered cell of any one of embodiments 1-45.

Embodiment 49 is a method of administering the engineered cell, population of cells, or pharmaceutical composition of any one of the preceding embodiments to a subject in need thereof.

Embodiment 50 is a method of administering the engineered cell, population of cells, or pharmaceutical composition of any one of the preceding embodiments to a subject as an adoptive cell transfer (ACT) therapy.

Embodiment 51 is an engineered cell, population of cells, or pharmaceutical composition of any one of the preceding embodiments, for use as an ACT therapy.

Embodiment 52 is a PD1 guide RNA that specifically hybridizes to a PD1 sequence, the guide RNA comprising a nucleotide sequence selected from:
a, a guide sequence comprising a nucleotide sequence selected from SEQ ID NOs: 1-88;
b, a guide sequence comprising a nucleotide sequence of at least 17, 18, 19, or 20 contiguous nucleotides of a nucleotide sequence selected from the sequence of SEQ ID NOs: 1-88;
c, a guide sequence comprising a nucleotide sequence at least 95% identical or at least 90% identical to a nucleotide sequence selected from SEQ ID Nos: 1-88;
d, a guide sequence comprising a nucleotide sequence selected from SEQ ID NOs: 5, 6, 8, 11, 12, 17, 22, 23, 24, 29, 36, 38, 41, 43, 56, 57 and 58;
e, a guide sequence comprising a nucleotide sequence selected from SEQ ID NOs: 5, 6, 8, 11, 12, 23, 24, 29, 36, 38, 43, and 57;
f, a guide sequence comprising a nucleotide sequence selected from SEQ ID NOs: 5, 11, 12, 22, 23, and 43;
g, a guide sequence comprising a nucleotide sequence selected from SEQ ID NOs: 6, 8, 23, and 29;
h, a guide sequence comprising a nucleotide sequence selected from SEQ ID NOs: 6 and 29;
i, a guide sequence comprising a nucleotide sequence selected from SEQ ID NOs: 6, 23, 29, 41, and 57;
j, a guide sequence comprising a nucleotide sequence selected from SEQ ID NOs: 6, 29, and 57; and
k, a guide sequence comprising a nucleotide sequence of SEQ ID NO: 43.

Embodiment 53 is a PD1 guide RNA comprising a guide sequence that directs an RNA-guided DNA binding agent to a chromosomal location within the genomic coordinates selected from those targeted by SEQ ID NOs: 1-88; SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43.

Embodiment 54 is the guide RNA of embodiments 52 or 53, wherein the guide RNA is a dual guide RNA (dgRNA).

Embodiment 55 is the guide RNA of embodiments 52 or 53, wherein the guide RNA is a single guide RNA (sgRNA).

Embodiment 56 is the guide RNA of embodiment 55, further comprising the nucleotide sequence of SEQ ID NO: 201 at the 3′ end to the guide sequence, wherein the guide RNA comprises a 5′ end modification or a 3′ end modification.

Embodiment 57 is the guide RNA of embodiment 55, further comprising 5′ end modification or a 3′ end modification and a conserved portion of an gRNA comprising one or more of:
A. a shortened hairpin 1 region or a substituted and optionally shortened hairpin 1 region relative to SEQ ID NO: 201, wherein
   1. at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to SEQ ID NO: 201 or
      b. one or more of positions H1-6 through H1-10 is substituted relative to SEQ ID NO: 201; or
   3, the shortened hairpin 1 region lacks 5-10 nucleotides, preferably 5-6 nucleotides, and one or more of positions N18, H1-12, or n is substituted relative to SEQ ID NO: 201; or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 201; or
C. a substitution relative to SEQ ID NO: 201 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an upper stem region, wherein the upper stem modification comprises a modification to any one or more of US1-US12 in the upper stem region.

Embodiment 58 is the guide RNA of embodiment 55, further comprising the nucleotide sequence of GUUUUAGAGCUAUGCUGUUUUG (SEQ ID NO: 200) 3′ to the guide sequence.

Embodiment 59 is the guide RNA of embodiment 55, further comprising the nucleotide sequence of GUUUUAGAGCUAGAAAUAGCAAGUUAAAAUAAGGCUAGUCCGUUAUCAACUU GAAAAAGUGGCACCGAGUCGGUGC (SEQ ID NO: 201) 3′ to the guide sequence, optionally GUUUUAGAGCUAGAAAUAGCAAGUUAAAAUAAGGCUAGUCCGUUAUCAACUU GAAAAAGUGGCACCGAGUCGGUGCUUUU (SEQ ID NO: 202) 3′ to the guide sequence.

Embodiment 60 is the guide RNA of embodiment 58 or 59, wherein the guide RNA is modified according to the pattern mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, m is a 2′-O-methyl modified nucleotide, and * is a phosphorothioate linkage between nucleotide residues; and wherein the N's are collectively the nucleotide sequence of a guide sequence of any preceding embodiment.

Embodiment 61 is the guide RNA of embodiment 60, wherein each N is independently any natural or non-natural nucleotide and the guide sequence targets Cas9 to the PD1 gene.

Embodiment 62 is the guide RNA of any one of embodiments 55-61, wherein the guide RNA comprises a modification.

Embodiment 63 is the guide RNA of embodiment 62, wherein the modification comprises a 2′-O-methyl (2′-O-Me) modified nucleotide or a 2′-F modified nucleotide.

Embodiment 64 is the guide RNA of embodiments 62-64, wherein the modification comprises a phosphorothioate (PS) bond between nucleotides.

Embodiment 65 is the guide RNA of any one of embodiments 62-64, wherein the guide RNA is a sgRNA and the modification, comprises a modification at one or more of the five nucleotides at the 5′ end of the guide RNA.

Embodiment 66 is the guide RNA of any one of embodiments 62-65, wherein the guide RNA is a sgRNA and the modification, comprises a modification at one or more of the five nucleotides at the 3′ end of the guide RNA.

Embodiment 67 is the guide RNA of any one of embodiments 62-66, wherein the guide RNA is a sgRNA and the modification, comprises a PS bond between each of the four nucleotides at the 5′ end of the guide RNA.

Embodiment 68 is the guide RNA of any one of embodiments 62-67, wherein the guide RNA is a sgRNA and the modification, comprises a PS bond between each of the four nucleotides at the 3′ end of the guide RNA.

Embodiment 69 is the guide RNA of any one of embodiments 62-68, wherein the guide RNA is a sgRNA and the modification, comprises a 2′-O-Me modified nucleotide at each of the first three nucleotides at the 5′ end of the guide RNA.

Embodiment 70 is the guide RNA of any one of embodiments 62-69, wherein the guide RNA is a sgRNA and the modification, comprises a 2′-O-Me modified nucleotide at each of the last three nucleotides at the 3′ end of the guide RNA.

Embodiment 71 is the composition comprising a guide RNA of any one of embodiments 52-70 and an RNA guided DNA binding agent wherein the RNA guided DNA binding agent is a polypeptide RNA guided DNA binding agent or a nucleic acid encoding an RNA guided DNA binding agent polypeptide, optionally the RNA guided DNA-binding agent is a Cas9 nuclease.

Embodiment 72 is the composition of embodiment 71, wherein the RNA guided DNA binding agent is a polypeptide capable of making a modification within a DNA sequence.

Embodiment 73 is the composition of embodiment 72, wherein the RNA guided DNA binding agent is a S, pyogenes Cas9 nuclease.

Embodiment 74 is the composition of any one of embodiments 71-73, wherein the nuclease is selected from the group of cleavase, nickase, and dead nuclease.

Embodiment 75 is the composition of embodiment 71, wherein the nucleic acid encoding an RNA guided DNA binding agent is selected from:
a. a DNA coding sequence;
b. an mRNA with an open reading frame (ORF);
c. a coding sequence in an expression vector;
d, a coding sequence in a viral vector.

Embodiment 76 is the composition of any one of embodiments 71-75 further comprising a guide RNA that specifically hybridizes to genomic coordinates chosen from:

| TRAC NO: | Genomic Coordinates (hg38) |
|---|---|
| TRAC-90 | chr14: 22547524-22547544 |
| TRAC-91 | chr14: 22550581-22550601 |
| TRAC-92 | chr14: 22550608-22550628 |
| TRAC-93 | chr14: 22550611-22550631 |
| TRAC-94 | chr14: 22550622-22550642 |
| TRAC-95 | chr14: 22547529-22547549 |
| TRAC-96 | chr14: 22547512-22547532 |
| TRAC-97 | chr14: 22547525-22547545 |
| TRAC-98 | chr14: 22547536-22547556 |
| TRAC-99 | chr14: 22547575-22547595 |
| TRAC-100 | chr14: 22547640-22547660 |
| TRAC-101 | chr14: 22547647-22547667 |
| TRAC-102 | chr14: 22547777-22547797 |
| TRAC-103 | chr14: 22549638-22549658 |
| TRAC-104 | chr14: 22549646-22549666 |
| TRAC-105 | chr14: 22550600-22550620 |
| TRAC-106 | chr14: 22550605-22550625 |
| TRAC-107 | chr14: 22550625-22550645 |
| TRAC-108 | chr14: 22539116-22539136 |
| TRAC-109 | chr14: 22539120-22539140 |
| TRAC-110 | chr14: 22547518-22547538 |
| TRAC-111 | chr14: 22539082-22539102 |
| TRAC-112 | chr14: 22539061-22539081 |
| TRAC-113 | chr14: 22539097-22539117 |
| TRAC-114 | chr14: 22547697-22547717 |
| TRAC-115 | chr14: 22550571-22550591 |
| TRAC-116 | chr14: 22550631-22550651 |
| TRAC-117 | chr14: 22550658-22550678 |
| TRAC-118 | chr14: 22547712-22547732 |
| TRAC-119 | chr14: 22550636-22550656 |
| TRAC-120 | chr14: 22550636-22550656 |
| TRAC-121 | chr14: 22550582-22550602 |
| TRAC-122 | chr14: 22550606-22550626 |
| TRAC-123 | chr14: 22550609-22550629 |
| TRAC-124 | chr14: 22547691-22547711 |
| TRAC-125 | chr14: 22547576-22547596 |
| TRAC-126 | chr14: 22549648-22549668 |
| TRAC-127 | chr14: 22549660-22549680 |
| TRAC-128 | chr14: 22547716-22547736 |
| TRAC-129 | chr14: 22547514-22547534 |
| TRAC-130 | chr14: 22550662-22550682 |
| TRAC-131 | chr14: 22550593-22550613 |
| TRAC-132 | chr14: 22550612-22550632 |
| TRAC-133 | chr14: 22547521-22547541 |
| TRAC-134 | chr14: 22547540-22547560 |
| TRAC-135 | chr14: 22539121-22539141 |
| TRAC-136 | chr14: 22547632-22547652 |
| TRAC-137 | chr14: 22547674-22547694 |
| TRAC-138 | chr14: 22549643-22549663 |
| TRAC-139 | chr14: 22547655-22547675 |
| TRAC-140 | chr14: 22547667-22547687 |
| TRAC-141 | chr14: 22539085-22539105 |
| TRAC-142 | chr14: 22549634-22549654 |
| TRAC-143 | chr14: 22539064-22539084 |
| TRAC-144 | chr14: 22547639-22547659 |
| TRAC-145 | chr14: 22547731-22547751 |
| TRAC-146 | chr14: 22547734-22547754 |
| TRAC-147 | chr14: 22547591-22547611 |
| TRAC-148 | chr14: 22547657-22547677 |
| TRAC-149 | chr14: 22547519-22547539 |
| TRAC-150 | chr14: 22549674-22549694 |
| TRAC-151 | chr14: 22547678-22547698 |
| TRAC-152 | chr14: 22539087-22539107 |
| TRAC-153 | chr14: 22547595-22547615 |
| TRAC-154 | chr14: 22547633-22547653 |
| TRAC-155 | chr14: 22547732-22547752 |
| TRAC-156 | chr14: 22547656-22547676 |
| TRAC-157 | chr14: 22539086-22539106 |
| TRAC-158 | chr14: 22547491-22547511 |
| TRAC-159 | chr14: 22547618-22547638 |
| TRAC-160 | chr14: 22549644-22549664 |
| TRAC-161 | chr14: 22547522-22547542 |
| TRAC-162 | chr14: 22539089-22539109 |
| TRAC-163 | chr14: 22539062-22539082 |
| TRAC-164 | chr14: 22547597-22547617 |
| TRAC-165 | chr14: 22547677-22547697 |
| TRAC-166 | chr14: 22549645-22549665 |
| TRAC-167 | chr14: 22550610-22550630 |
| TRAC-168 | chr14: 22547511-22547531 |
| TRAC-169 | chr14: 22550607-22550627 |
| TRAC-170 | chr14: 22550657-22550677 |
| TRAC-171 | chr14: 22550604-22550624 |
| TRAC-172 | chr14: 22539132-22539152 |
| TRAC-173 | chr14: 22550632-22550652 |
| TRAC-174 | chr14: 22547571-22547591 |
| TRAC-175 | chr14: 22547711-22547731 |
| TRAC-176 | chr14: 22547666-22547686 |
| TRAC-177 | chr14: 22547567-22547587 |
| TRAC-178 | chr14: 22547624-22547644 |
| TRAC-185 | chr14: 22547501-22547521 |
| TRAC-213 | chr14: 22547519-22547539 |
| TRAC-214 | chr14: 22547556-22547576 |
| TRAC-215 | chr14: 22547486-22547506 |
| TRAC-216 | chr14: 22547487-22547507 |
| TRAC-217 | chr14: 22547493-22547513 |
| TRAC-218 | chr14: 22547502-22547522 |

optionally wherein the genetic modification is within genomic coordinates selected from chr14:22547524-22547544, chr14:22547529-22547549, chr14:22547525-22547545, chr14:22547536-22547556, chr14:22547501-22547521, chr14:22547556-22547576, and chr14:22547502-22547522.

Embodiment 77 is the composition of any one of embodiments 71-76 further comprising a guide RNA that specifically hybridizes to genomic coordinates chosen from:

| TRBC NO: | Genomic Coordinates (hg38) |
|---|---|
| TRBC-1 | chr7: 142791996-142792016 |
| TRBC-2 | chr7: 142792047-142792067 |
| TRBC-3 | chr7: 142792008-142792028 |
| TRBC-4 | chr7: 142791931-142791951 |
| TRBC-5 | chr7: 142791930-142791950 |
| TRBC-6 | chr7: 142791748-142791768 |
| TRBC-7 | chr7: 142791720-142791740 |
| TRBC-8 | chr7: 142792041-142792061 |
| TRBC-9 | chr7: 142802114-142802134 |
| TRBC-10 | chr7: 142792009-142792029 |
| TRBC-11 | chr7: 142792697-142792717 |
| TRBC-12 | chr7: 142791963-142791983 |
| TRBC-13 | chr7: 142791976-142791996 |
| TRBC-14 | chr7: 142791974-142791994 |
| TRBC-15 | chr7: 142791970-142791990 |
| TRBC-16 | chr7: 142791948-142791968 |
| TRBC-17 | chr7: 142791913-142791933 |
| TRBC-18 | chr7: 142791961-142791981 |
| TRBC-19 | chr7: 142792068-142792088 |
| TRBC-20 | chr7: 142791975-142791995 |
| TRBC-21 | chr7: 142791773-142791793 |
| TRBC-22 | chr7: 142791919-142791939 |
| TRBC-23 | chr7: 142791834-142791854 |
| TRBC-24 | chr7: 142791878-142791898 |
| TRBC-25 | chr7: 142802141-142802161 |
| TRBC-26 | chr7: 142791844-142791864 |
| TRBC-27 | chr7: 142801154-142801174 |
| TRBC-28 | chr7: 142791961-142791981 |
| TRBC-29 | chr7: 142792001-142792021 |
| TRBC-30 | chr7: 142791979-142791999 |
| TRBC-31 | chr7: 142792041-142792061 |
| TRBC-32 | chr7: 142792003-142792023 |
| TRBC-33 | chr7: 142791984-142792004 |
| TRBC-34 | chr7: 142792002-142792022 |
| TRBC-35 | chr7: 142791966-142791986 |
| TRBC-36 | chr7: 142792007-142792027 |
| TRBC-37 | chr7: 142791993-142792013 |
| TRBC-38 | chr7: 142791902-142791922 |
| TRBC-39 | chr7: 142791724-142791744 |
| TRBC-40 | chr7: 142791973-142791993 |
| TRBC-41 | chr7: 142791920-142791940 |
| TRBC-42 | chr7: 142791994-142792014 |
| TRBC-43 | chr7: 142791887-142791907 |
| TRBC-44 | chr7: 142791907-142791927 |
| TRBC-45 | chr7: 142791952-142791972 |
| TRBC-46 | chr7: 142791721-142791741 |
| TRBC-47 | chr7: 142792718-142792738 |
| TRBC-48 | chr7: 142791729-142791749 |
| TRBC-49 | chr7: 142791911-142791931 |
| TRBC-50 | chr7: 142791867-142791887 |
| TRBC-51 | chr7: 142791899-142791919 |
| TRBC-52 | chr7: 142791727-142791747 |
| TRBC-53 | chr7: 142791949-142791969 |
| TRBC-54 | chr7: 142791933-142791953 |
| TRBC-55 | chr7: 142791932-142791952 |
| TRBC-56 | chr7: 142792057-142792077 |
| TRBC-57 | chr7: 142791940-142791960 |
| TRBC-58 | chr7: 142791747-142791767 |
| TRBC-59 | chr7: 142791881-142791901 |
| TRBC-60 | chr7: 142791779-142791799 |
| TRBC-61 | chr7: 142792054-142792074 |
| TRBC-62 | chr7: 142792069-142792089 |
| TRBC-63 | chr7: 142792712-142792732 |
| TRBC-64 | chr7: 142791729-142791749 |
| TRBC-65 | chr7: 142791821-142791841 |
| TRBC-66 | chr7: 142792052-142792072 |
| TRBC-67 | chr7: 142791916-142791936 |
| TRBC-68 | chr7: 142791899-142791919 |
| TRBC-69 | chr7: 142791772-142791792 |
| TRBC-70 | chr7: 142792714-142792734 |
| TRBC-71 | chr7: 142792042-142792062 |
| TRBC-72 | chr7: 142791962-142791982 |
| TRBC-73 | chr7: 142791988-142792008 |
| TRBC-74 | chr7: 142791982-142792002 |
| TRBC-75 | chr7: 142792049-142792069 |
| TRBC-76 | chr7: 142791839-142791859 |
| TRBC-77 | chr7: 142791893-142791913 |
| TRBC-78 | chr7: 142791945-142791965 |
| TRBC-79 | chr7: 142791964-142791984 |
| TRBC-80 | chr7: 142791757-142791777 |
| TRBC-81 | chr7: 142792048-142792068 |
| TRBC-82 | chr7: 142791774-142791794 |
| TRBC-83 | chr7: 142792048-142792068 |
| TRBC-84 | chr7: 142791830-142791850 |
| TRBC-85 | chr7: 142791909-142791929 |
| TRBC-86 | chr7: 142791912-142791932 |
| TRBC-87 | chr7: 142791766-142791786 |
| TRBC-88 | chr7: 142791880-142791900 |
| TRBC-89 | chr7: 142791919-142791939 |

Embodiment 78 is the composition of any one of embodiments 71-77 further comprising a guide RNA that specifically hybridizes to genomic coordinates chosen from chr: 16:10902171-10923242, optionally, chr16:10902662-chr16:10923285, chr16:10906542-chr16:10923285, or chr16:10906542-chr16:10908121, optionally chr16:10908132-10908152, chr16:10908131-10908151, chr16:10916456-10916476, chr16:10918504-10918524, chr16:10918511-10918531, chr16:10916455-10916475, chr16:10895742-10895762, chr16:10916362-10916382, chr16:10909006-10909026, chr16:10909172-10909192, chr16:10906492-10906512, chr16:10916348-10916368, chr16:10922478-10922498, chr16:10895747-10895767, chr16:10909007-10909027, chr16:10910186-10910206, chr16:10906481-10906501, chr16:10895410-10895430, and chr16:10908130-10908150; optionally chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-chr16:10909022-10909042, chr16:10918512-10918532, 10916469, chr16:10923214-10923234, chr16:10906492-10906512, and chr16:10906487-1090650; or optionally chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907810-10907830, chr16:10907119-10907139, chr16:10907539-10907559, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

Embodiment 79 is the composition of any one of embodiments 71-77 further comprising a guide RNA that specifically hybridizes to genomic coordinates chosen from chr6:29942854-29942913 and chr6:29943518-29943619, optionally genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046

Embodiment 80 is the guide RNA of any one of embodiments 52-70 or the composition of any one of any one of embodiments 71-79, wherein the composition further comprises a pharmaceutically acceptable excipient.

Embodiment 81 is the guide or composition of embodiment 80, wherein the composition is non-pyrogenic.

Embodiment 82 is the guide RNA of any one of embodiments 52-70 or composition of any one of embodiments 71-81, wherein the guide RNA is associated with a lipid nanoparticle (LNP).

Embodiment 83 is a method of making a genetic modification in a PD1 sequence within a cell, comprising contacting the cell with the guide RNA or composition of any one of embodiments 52-82.

Embodiment 84 is the method of embodiment 83, further comprising making a genetic modification in a TCR sequence to inhibit expression of a TCR gene.

Embodiment 85 is a method of preparing a population of cells for immunotherapy comprising:
a. making a genetic modification in a PD1 sequence in the cells in the population with a PD1 guide RNA or composition of any one of embodiments 52-82;
b. making a genetic modification in a TCR sequence in the cells of the population to reduce expression of the TCR protein on the surface of the cells in the population;
c. expanding the population of cells in culture.

Embodiment 86 is the method of embodiment 85, wherein expression of the TCR protein on the surface of the cells is reduced to below the level of detection in at least 40%, 45%, 50%, 55%, 60%, 65%, preferably at least 70%, 75%, 80%, 85%, 90%, or 95% of the cells in the population.

Embodiment 87 is the method of embodiments 85 or 86, wherein the genetic modification of a TCR sequence in the cells of the population comprises modification of two or more TCR sequences.

Embodiment 88 is the method of embodiments 87 wherein the two or more TCR sequences comprise TRAC and TRBC.

Embodiment 89 is the method of any of embodiments 85-88, comprising insertion of an exogenous nucleic acid encoding a targeting receptor that is expressed on the surface of the engineered cell, e.g. a TCR or a CAR, optionally at a TRAC locus.

Embodiment 90 is the method of any one of embodiments 85-89, further comprising contacting the cells with an LNP composition comprising the PD1 guide RNA.

Embodiment 91 is the method of embodiment 90 comprising contacting the cells with a second LNP composition comprising a guide RNA.

Embodiment 92 is a population of cells made by the method of any one of embodiments 83-91.

Embodiment 93 is the population of cells of embodiment 92, wherein the population of cells is altered ex vivo.

Embodiment 94 is a pharmaceutical composition comprising a population of cells of embodiments 92 or 93.

Embodiment 95 is a method of administering the population of cells of embodiments 92 or 93, or pharmaceutical composition of embodiment 94 to a subject in need thereof.

Embodiment 96 is a method of administering the population of cells of embodiments 92 or 93, or pharmaceutical composition of embodiment 94 to a subject as an adoptive cell transfer (ACT) therapy.

Embodiment 97 is a population of cells of embodiments 92 or 93, or pharmaceutical composition of embodiment 94, for use as an ACT therapy.

Embodiment 98 is a population of cells comprising a genetic modification of a PD1 gene, wherein at least 40%, 45%, 50%, 55%, 60%, 65%, preferably at least 70%, 75%, 80%, 85%, 90%, or 95% of cells in the population comprise a modification selected from an insertion, a deletion, and a substitution in the endogenous PD1 sequence.

Embodiment 99 is the population of cells of embodiment 98, wherein the genetic modification is as defined in any one of embodiment 1-4.

Embodiment 100 is the population of cells of embodiments 98 or 99, wherein expression of PD1 is decreased by at least 40%, 45%, 50%, 55%, 60%, 65%, preferably at least 70%, 75%, 80%, 85%, 90%, 95%, or to below the limit of detection of the assay as compared to a suitable control, e.g., wherein the PD1 gene has not been modified.

Embodiment 101 is a population of cells comprising a genetic modification of a TCR gene, wherein at least 40%, 45%, 50%, 55%, 60%, 65%, preferably at least 70%, 75%, 80%, 85%, 90%, 95%, 96%, 97%, 98%, or 99% of cells comprise a modification selected from an insertion, a deletion, and a substitution in the endogenous TCR gene sequence.

Embodiment 102 is the population of cells of embodiment 101, wherein the genetic modification is as defined in any of embodiments 5-8.

Embodiment 103 is the population of cells of embodiments 101 or 102, wherein expression of TCR is decreased by at least 40%, 45%, 50%, 55%, 60%, 65%, preferably at least 70%, 75%, 80%, 85%, 90%, 95%, 96%, 97%, 98%, 99%, or to below the limit of detection of the assay as compared to a suitable control, e.g., wherein the TCR gene has not been modified.

Embodiment 104 is the population of cells of any of embodiments 98-103, wherein the population comprises at least 10³, 10⁴, 10⁵ or 10⁶ cells, preferably 10⁷, 2×10⁷, 5×10⁷, or 10⁸ cells.

Embodiment 105 is the population of cells of any one of embodiments 98-104, wherein at least 70% of cells in the population comprise a modification selected from an insertion, a deletion, and a substitution in the endogenous PD1 sequence.

Embodiment 106 is the population of cells of any one of embodiments 98-105, wherein at least 80% of cells in the population comprise a modification selected from an insertion, a deletion, and a substitution in the endogenous PD1 sequence.

Embodiment 107 is the population of cells of any one of embodiments 98-106, wherein at least 90% of cells in the population comprise a modification selected from an insertion, a deletion, and a substitution in the endogenous PD1 sequence.

Embodiment 108 is the population of cells of any one of embodiments 98-107, wherein at least 95% of cells in the population comprise a modification selected from an insertion, a deletion, and a substitution in the endogenous PD1 sequence.

Embodiment 109 is the population of cells of any one of embodiments 98-108, wherein expression of PD1 is decreased by at least 70%, or to below the limit of detection of the assay as compared to a suitable control, e.g., wherein the PD1 gene has not been modified.

Embodiment 110 is the population of cells of any one of embodiments 98-109, wherein expression of PD1 is decreased by at least 80%, or to below the limit of detection of the assay as compared to a suitable control, e.g., wherein the PD1 gene has not been modified.

Embodiment 111 is the population of cells of any one of embodiments 98-110, wherein expression of PD1 is decreased by at least 90%, or to below the limit of detection of the assay as compared to a suitable control, e.g., wherein the PD1 gene has not been modified.

Embodiment 112 is the population of cells of any one of embodiments 98-111, wherein expression of PD1 is decreased by at least 95%, or to below the limit of detection of the assay as compared to a suitable control, e.g., wherein the PD1 gene has not been modified.

Embodiment 113 is a pharmaceutical composition comprising the population of cells of any of embodiments 98-112.

Embodiment 114 is the population of cells of any of embodiments 98-113 or the pharmaceutical composition of embodiment 113, for use as an ACT therapy.

Embodiment 115 is an engineered cell, guide RNA, composition, pharmaceutical composition, or method of any one of the preceding embodiments, wherein the genetic modification is within the genomic coordinates of chr2:241852703-241852723.

Embodiment 116 is the engineered cell, guide RNA, composition, pharmaceutical composition, or method of any one of the preceding embodiments, wherein the genetic modification is within the genomic coordinates of chr2:241858807-241858827.

Embodiment 117 is the engineered cell, guide RNA, composition, pharmaceutical composition, or method of any one of the preceding embodiments, wherein the genetic modification is within the genomic coordinates of chr2:241858789-241858809.

Embodiment 118 is the engineered cell, guide RNA, composition, pharmaceutical composition, or method of any one of the preceding embodiments, wherein the genetic modification is within the genomic coordinates of chr2:241858788-241858808.

Embodiment 119 is the engineered cell, guide RNA, composition, pharmaceutical composition, or method of any one of the preceding embodiments, wherein the genetic modification is within the genomic coordinates of chr2:241858755-241858775.

Embodiment 120 is the engineered cell, guide RNA, composition, pharmaceutical composition, or method of any one of the preceding embodiments, wherein the genetic modification is within the genomic coordinates of chr2:241852919-241852939.

Embodiment 121 is the engineered cell, guide RNA, composition, pharmaceutical composition, or method of any one of the preceding embodiments, wherein the genetic modification is within the genomic coordinates of chr2:241852915-241852935.

Embodiment 122 is the engineered cell, guide RNA, composition, pharmaceutical composition, or method of any one of the preceding embodiments, wherein the genetic modification is within the genomic coordinates of chr2:241852755-241852775.

Embodiment 123 is the engineered cell, guide RNA, composition, pharmaceutical composition, or method of any one of the preceding embodiments, wherein the genetic modification is within the genomic coordinates of chr2:241852751-241852771.

Embodiment 124 is the engineered cell, guide RNA, composition, pharmaceutical composition, or method of any one of the preceding embodiments, wherein the genetic modification is within the genomic coordinates of chr2:241852750-241852770.

Embodiment 125 is the engineered cell, guide RNA, composition, pharmaceutical composition, or method of any one of the preceding embodiments, wherein the genetic modification is within the genomic coordinates of chr2:241852264-241852284.

Embodiment 126 is the engineered cell, guide RNA, composition, pharmaceutical composition, or method of any one of the preceding embodiments, wherein the genetic modification is within the genomic coordinates of chr2:241852201-241852221.

Embodiment 127 is the engineered cell, guide RNA, composition, pharmaceutical composition, or method of any one of the preceding embodiments, wherein the genetic modification is within the genomic coordinates of chr2:241852749-241852769.

Embodiment 128 is the engineered cell, guide RNA, composition, pharmaceutical composition, or method of any one of the preceding embodiments, wherein the genetic modification is within the genomic coordinates of chr2:241852821-241852841.

Embodiment 129 is the engineered cell, guide RNA, composition, pharmaceutical composition, or method of any one of the preceding embodiments, wherein the genetic modification is within the genomic coordinates of chr2:241852265-241852285.

Embodiment 130 is the engineered cell, guide RNA, composition, pharmaceutical composition, or method of any one of the preceding embodiments, wherein the genetic modification is within the genomic coordinates of chr2:241851221-241851241.

Embodiment 131 is the engineered cell, guide RNA, composition, pharmaceutical composition, or method of any one of the preceding embodiments, wherein the genetic modification is within the genomic coordinates of chr2:241852188-241852208.

What is claimed is:

1. An engineered cell comprising a genetic modification in a human PD1 sequence, within genomic coordinates of chr2: 241849881-241858908.

2. The engineered cell of claim 1, wherein the genetic modification is selected from an insertion, a deletion, and a substitution.

3. The engineered cell of claim 1 or 2, wherein the genetic modification inhibits expression of the PD1 gene.

4. The engineered cell of any one of claims 1-3, wherein the genetic modification comprises a modification of at least one nucleotide within the genomic coordinates selected from:

| PD1 NO. | Genomic Coordinates (hg38) |
|---|---|
| PD1-29 | chr2: 241852703-241852723 |
| PD1-43 | chr2: 241858807-241858827 |
| PD1-5 | chr2: 241858789-241858809 |
| PD1-6 | chr2: 241858788-241858808 |
| PD1-8 | chr2: 241858755-241858775 |
| PD1-11 | chr2: 241852919-241852939 |
| PD1-12 | chr2: 241852915-241852935 |
| PD1-22 | chr2: 241852755-241852775 |
| PD1-23 | chr2: 241852751-241852771 |
| PD1-24 | chr2: 241852750-241852770 |
| PD1-36 | chr2: 241852264-241852284 |
| PD1-57 | chr2: 241852201-241852221 |
| PD1-58 | chr2: 241852749-241852769 |
| PD1-17 | chr2: 241852821-241852841 |
| PD1-38 | chr2: 241852265-241852285 |
| PD1-56 | chr2: 241851221-241851241 |
| PD1-41 | chr2: 241852188-241852208; |

or
the genomic coordinates selected from those targeted by PD1-11, PD1-12, PD1-24, PD1-36, PD1-38, PD1-43, PD1-57, PD1-5, PD1-6, PD1-8, PD1-22, PD1-23, and PD1-29; chr2:241852919-241852939; chr2:241852915-241852935; chr2:241852750-241852770; chr2:241852264-241852284; chr2:241852265-241852285; chr2:241858807-241858827; chr2:241852201-241852221; chr2:241858789-241858809; chr2:241858788-241858808; chr2:241858755-241858775; chr2:241852755-241852775; chr2:241852751-241852771; and chr2:241852703-241852723; or
the genomic coordinates selected from those targeted by PD1-6, PD1-8, PD1-11, PD1-12, PD1-23, PD1-43, and PD1-29; chr2:241858788-241858808; chr2:241858755-241858775; chr2:241852919-241852939; chr2:241852915-241852935; chr2:241852751-241852771; chr2:241858807-241858827; and chr2:241852703-241852723; or
the genomic coordinates selected from those targeted by PD1-5, PD1-11, PD1-12, PD1-22, PD1-23, and PD1-43; chr2:241858789-241858809; chr2:241852919-241852939; chr2:241852915-241852935; chr2:241852755-241852775; chr2:241852751-241852771; and chr2:241858807-241858827; or
the genomic coordinates selected from those targeted by PD1-6, PD1-8, PD1-23, and PD1-29; chr2:241858788-241858808; chr2:241858755-241858775; chr2:241852751-241852771; and chr2:241852703-241852723; or
the genomic coordinates selected from those targeted by PD1-6 and PD1-29; chr2:241858788-241858808; and chr2:241852703-241852723; or
the genomic coordinates selected from those targeted by PD1-6, PD1-23, PD1-29, PD1-41, and PD1-57; chr2:241858788-241858808; chr2:241852751-241852771; chr2:241852703-241852723; chr2:241852188-241852208; and chr2:241852201-241852221; or
the genomic coordinates selected from those targeted by PD1-6, PD1-29, and PD1-57: chr2:241858788-241858808; chr2:241852703-241852723; and chr2:241852201-241852221; or
the genomic coordinates of PD1-43; chr2:241858807-241858827.

5. The engineered cell of any one of claims 1-4, wherein the engineered cell comprises a genetic modification within the genomic coordinates of an endogenous T cell receptor (TCR) sequence, wherein the genetic modification inhibits expression of the TCR gene, optionally wherein the TCR gene is TRAC or TRBC.

6. The engineered cell of claim 5, comprising a genetic modification of TRBC within genomic coordinates selected from:

| TRBC NO: | Genomic Coordinates (hg38) |
|---|---|
| TRBC-1 | chr7: 142791996-142792016 |
| TRBC-2 | chr7: 142792047-142792067 |
| TRBC-3 | chr7: 142792008-142792028 |
| TRBC-4 | chr7: 142791931-142791951 |
| TRBC-5 | chr7: 142791930-142791950 |
| TRBC-6 | chr7: 142791748-142791768 |
| TRBC-7 | chr7: 142791720-142791740 |
| TRBC-8 | chr7: 142792041-142792061 |
| TRBC-9 | chr7: 142802114-142802134 |
| TRBC-10 | chr7: 142792009-142792029 |
| TRBC-11 | chr7: 142792697-142792717 |
| TRBC-12 | chr7: 142791963-142791983 |
| TRBC-13 | chr7: 142791976-142791996 |
| TRBC-14 | chr7: 142791974-142791994 |
| TRBC-15 | chr7: 142791970-142791990 |
| TRBC-16 | chr7: 142791948-142791968 |
| TRBC-17 | chr7: 142791913-142791933 |
| TRBC-18 | chr7: 142791961-142791981 |
| TRBC-19 | chr7: 142792068-142792088 |
| TRBC-20 | chr7: 142791975-142791995 |
| TRBC-21 | chr7: 142791773-142791793 |
| TRBC-22 | chr7: 142791919-142791939 |
| TRBC-23 | chr7: 142791834-142791854 |
| TRBC-24 | chr7: 142791878-142791898 |
| TRBC-25 | chr7: 142802141-142802161 |
| TRBC-26 | chr7: 142791844-142791864 |
| TRBC-27 | chr7: 142801154-142801174 |
| TRBC-28 | chr7: 142791961-142791981 |
| TRBC-29 | chr7: 142792001-142792021 |
| TRBC-30 | chr7: 142791979-142791999 |
| TRBC-31 | chr7: 142792041-142792061 |
| TRBC-32 | chr7: 142792003-142792023 |
| TRBC-33 | chr7: 142791984-142792004 |
| TRBC-34 | chr7: 142792002-142792022 |
| TRBC-35 | chr7: 142791966-142791986 |
| TRBC-36 | chr7: 142792007-142792027 |
| TRBC-37 | chr7: 142791993-142792013 |
| TRBC-38 | chr7: 142791902-142791922 |
| TRBC-39 | chr7: 142791724-142791744 |
| TRBC-40 | chr7: 142791973-142791993 |
| TRBC-41 | chr7: 142791920-142791940 |
| TRBC-42 | chr7: 142791994-142792014 |
| TRBC-43 | chr7: 142791887-142791907 |
| TRBC-44 | chr7: 142791907-142791927 |
| TRBC-45 | chr7: 142791952-142791972 |
| TRBC-46 | chr7: 142791721-142791741 |
| TRBC-47 | chr7: 142792718-142792738 |
| TRBC-48 | chr7: 142791729-142791749 |
| TRBC-49 | chr7: 142791911-142791931 |
| TRBC-50 | chr7: 142791867-142791887 |
| TRBC-51 | chr7: 142791899-142791919 |
| TRBC-52 | chr7: 142791727-142791747 |
| TRBC-53 | chr7: 142791949-142791969 |
| TRBC-54 | chr7: 142791933-142791953 |
| TRBC-55 | chr7: 142791932-142791952 |
| TRBC-56 | chr7: 142792057-142792077 |
| TRBC-57 | chr7: 142791940-142791960 |
| TRBC-58 | chr7: 142791747-142791767 |
| TRBC-59 | chr7: 142791881-142791901 |
| TRBC-60 | chr7: 142791779-142791799 |
| TRBC-61 | chr7: 142792054-142792074 |
| TRBC-62 | chr7: 142792069-142792089 |
| TRBC-63 | chr7: 142792712-142792732 |
| TRBC-64 | chr7: 142791729-142791749 |
| TRBC-65 | chr7: 142791821-142791841 |
| TRBC-66 | chr7: 142792052-142792072 |
| TRBC-67 | chr7: 142791916-142791936 |
| TRBC-68 | chr7: 142791899-142791919 |
| TRBC-69 | chr7: 142791772-142791792 |
| TRBC-70 | chr7: 142792714-142792734 |
| TRBC-71 | chr7: 142792042-142792062 |
| TRBC-72 | chr7: 142791962-142791982 |
| TRBC-73 | chr7: 142791988-142792008 |
| TRBC-74 | chr7: 142791982-142792002 |
| TRBC-75 | chr7: 142792049-142792069 |
| TRBC-76 | chr7: 142791839-142791859 |
| TRBC-77 | chr7: 142791893-142791913 |
| TRBC-78 | chr7: 142791945-142791965 |
| TRBC-79 | chr7: 142791964-142791984 |
| TRBC-80 | chr7: 142791757-142791777 |
| TRBC-81 | chr7: 142792048-142792068 |
| TRBC-82 | chr7: 142791774-142791794 |
| TRBC-83 | chr7: 142792048-142792068 |
| TRBC-84 | chr7: 142791830-142791850 |
| TRBC-85 | chr7: 142791909-142791929 |
| TRBC-86 | chr7: 142791912-142791932 |
| TRBC-87 | chr7: 142791766-142791786 |
| TRBC-88 | chr7: 142791880-142791900 |
| TRBC-89 | chr7: 142791919-142791939 |

7. The engineered cell of any one of claims 5-6, comprising a genetic modification of TRAC within genomic coordinates selected from:

| TRAC NO: | Genomic Coordinates (hg38) |
|---|---|
| TRAC-90 | chr14: 22547524-22547544 |
| TRAC-91 | chr14: 22550581-22550601 |
| TRAC-92 | chr14: 22550608-22550628 |
| TRAC-93 | chr14: 22550611-22550631 |
| TRAC-94 | chr14: 22550622-22550642 |
| TRAC-95 | chr14: 22547529-22547549 |
| TRAC-96 | chr14: 22547512-22547532 |
| TRAC-97 | chr14: 22547525-22547545 |
| TRAC-98 | chr14: 22547536-22547556 |
| TRAC-99 | chr14: 22547575-22547595 |
| TRAC-100 | chr14: 22547640-22547660 |
| TRAC-101 | chr14: 22547647-22547667 |
| TRAC-102 | chr14: 22547777-22547797 |
| TRAC-103 | chr14: 22549638-22549658 |
| TRAC-104 | chr14: 22549646-22549666 |
| TRAC-105 | chr14: 22550600-22550620 |
| TRAC-106 | chr14: 22550605-22550625 |
| TRAC-107 | chr14: 22550625-22550645 |
| TRAC-108 | chr14: 22539116-22539136 |
| TRAC-109 | chr14: 22539120-22539140 |
| TRAC-110 | chr14: 22547518-22547538 |
| TRAC-111 | chr14: 22539082-22539102 |
| TRAC-112 | chr14: 22539061-22539081 |
| TRAC-113 | chr14: 22539097-22539117 |
| TRAC-114 | chr14: 22547697-22547717 |
| TRAC-115 | chr14: 22550571-22550591 |
| TRAC-116 | chr14: 22550631-22550651 |
| TRAC-117 | chr14: 22550658-22550678 |
| TRAC-118 | chr14: 22547712-22547732 |
| TRAC-119 | chr14: 22550636-22550656 |
| TRAC-120 | chr14: 22550636-22550656 |
| TRAC-121 | chr14: 22550582-22550602 |
| TRAC-122 | chr14: 22550606-22550626 |
| TRAC-123 | chr14: 22550609-22550629 |
| TRAC-124 | chr14: 22547691-22547711 |
| TRAC-125 | chr14: 22547576-22547596 |
| TRAC-126 | chr14: 22549648-22549668 |
| TRAC-127 | chr14: 22549660-22549680 |
| TRAC-128 | chr14: 22547716-22547736 |
| TRAC-129 | chr14: 22547514-22547534 |
| TRAC-130 | chr14: 22550662-22550682 |
| TRAC-131 | chr14: 22550593-22550613 |
| TRAC-132 | chr14: 22550612-22550632 |
| TRAC-133 | chr14: 22547521-22547541 |
| TRAC-134 | chr14: 22547540-22547560 |
| TRAC-135 | chr14: 22539121-22539141 |
| TRAC-136 | chr14: 22547632-22547652 |
| TRAC-137 | chr14: 22547674-22547694 |
| TRAC-138 | chr14: 22549643-22549663 |
| TRAC-139 | chr14: 22547655-22547675 |
| TRAC-140 | chr14: 22547667-22547687 |
| TRAC-141 | chr14: 22539085-22539105 |
| TRAC-142 | chr14: 22549634-22549654 |
| TRAC-143 | chr14: 22539064-22539084 |
| TRAC-144 | chr14: 22547639-22547659 |
| TRAC-145 | chr14: 22547731-22547751 |
| TRAC-146 | chr14: 22547734-22547754 |
| TRAC-147 | chr14: 22547591-22547611 |
| TRAC-148 | chr14: 22547657-22547677 |
| TRAC-149 | chr14: 22547519-22547539 |
| TRAC-150 | chr14: 22549674-22549694 |
| TRAC-151 | chr14: 22547678-22547698 |
| TRAC-152 | chr14: 22539087-22539107 |
| TRAC-153 | chr14: 22547595-22547615 |
| TRAC-154 | chr14: 22547633-22547653 |
| TRAC-155 | chr14: 22547732-22547752 |
| TRAC-156 | chr14: 22547656-22547676 |
| TRAC-157 | chr14: 22539086-22539106 |
| TRAC-158 | chr14: 22547491-22547511 |
| TRAC-159 | chr14: 22547618-22547638 |
| TRAC-160 | chr14: 22549644-22549664 |
| TRAC-161 | chr14: 22547522-22547542 |
| TRAC-162 | chr14: 22539089-22539109 |
| TRAC-163 | chr14: 22539062-22539082 |
| TRAC-164 | chr14: 22547597-22547617 |
| TRAC-165 | chr14: 22547677-22547697 |
| TRAC-166 | chr14: 22549645-22549665 |
| TRAC-167 | chr14: 22550610-22550630 |
| TRAC-168 | chr14: 22547511-22547531 |
| TRAC-169 | chr14: 22550607-22550627 |
| TRAC-170 | chr14: 22550657-22550677 |
| TRAC-171 | chr14: 22550604-22550624 |
| TRAC-172 | chr14: 22539132-22539152 |
| TRAC-173 | chr14: 22550632-22550652 |
| TRAC-174 | chr14: 22547571-22547591 |
| TRAC-175 | chr14: 22547711-22547731 |
| TRAC-176 | chr14: 22547666-22547686 |
| TRAC-177 | chr14: 22547567-22547587 |
| TRAC-178 | chr14: 22547624-22547644 |
| TRAC-185 | chr14: 22547501-22547521 |
| TRAC-213 | chr14: 22547519-22547539 |
| TRAC-214 | chr14: 22547556-22547576 |
| TRAC-215 | chr14: 22547486-22547506 |
| TRAC-216 | chr14: 22547487-22547507 |
| TRAC-217 | chr14: 22547493-22547513 |
| TRAC-218 | chr14: 22547502-22547522 |

or wherein the genetic modification is within the genomic coordinates selected from chr14:22547524-22547544, chr14:22547529-22547549, chr14:22547525-22547545, chr14:22547536-22547556, chr14:22547501-22547521, chr14:22547556-22547576, and chr14:22547502-22547522.

8. The engineered cell of any one of claims 1-7, wherein the cell comprises a genetic modification, wherein the genetic modification inhibits expression of one or more MHC class I proteins.

9. The engineered cell of claim 8, wherein the genetic modification that inhibits expression of one or more MHC class I proteins is a genetic modification in a B2M sequence, wherein the genetic modification is within genomic coordinates selected from:

| B2M NO: | Genomic Location (hg38) |
|---|---|
| B2M-1 | chr15: 44711469-44711494 |
| B2M-2 | chr15: 44711472-44711497 |
| B2M-3 | chr15: 44711483-44711508 |
| B2M-4 | chr15: 44711486-44711511 |
| B2M-5 | chr15: 44711487-44711512 |
| B2M-6 | chr15: 44711512-44711537 |
| B2M-7 | chr15: 44711513-44711538 |
| B2M-8 | chr15: 44711534-44711559 |
| B2M-9 | chr15: 44711568-44711593 |
| B2M-10 | chr15: 44711573-44711598 |
| B2M-11 | chr15: 44711576-44711601 |
| B2M-12 | chr15: 44711466-44711491 |
| B2M-13 | chr15: 44711522-44711547 |
| B2M-14 | chr15: 44711544-44711569 |
| B2M-15 | chr15: 44711559-44711584 |
| B2M-16 | chr15: 44711565-44711590 |
| B2M-17 | chr15: 44711599-44711624 |
| B2M-18 | chr15: 44711611-44711636 |
| B2M-19 | chr15: 44715412-44715437 |
| B2M-20 | chr15: 44715440-44715465 |
| B2M-21 | chr15: 44715473-44715498 |
| B2M-22 | chr15: 44715474-44715499 |
| B2M-23 | chr15: 44715515-44715540 |
| B2M-24 | chr15: 44715535-44715560 |
| B2M-25 | chr15: 44715562-44715587 |
| B2M-26 | chr15: 44715567-44715592 |
| B2M-27 | chr15: 44715672-44715697 |
| B2M-28 | chr15: 44715673-44715698 |
| B2M-29 | chr15: 44715674-44715699 |
| B2M-30 | chr15: 44715410-44715435 |
| B2M-31 | chr15: 44715411-44715436 |
| B2M-32 | chr15: 44715419-44715444 |
| B2M-33 | chr15: 44715430-44715455 |
| B2M-34 | chr15: 44715457-44715482 |
| B2M-35 | chr15: 44715483-44715508 |
| B2M-36 | chr15: 44715511-44715536 |
| B2M-37 | chr15: 44715515-44715540 |
| B2M-38 | chr15: 44715629-44715654 |
| B2M-39 | chr15: 44715630-44715655 |
| B2M-40 | chr15: 44715631-44715656 |
| B2M-41 | chr15: 4471S632-44715657 |
| B2M-42 | chr15: 44715653-44715678 |
| B2M-43 | chr15: 44715657-44715682 |
| B2M-44 | chr15: 44715666-44715691 |
| B2M-45 | chr15: 44715685-44715710 |
| B2M-46 | chr15: 44715686-44715711 |
| B2M-47 | chr15: 44716326-44716351 |
| B2M-48 | chr15: 44716329-44716354 |
| B2M-49 | chr15: 44716313-44716338 |
| B2M-50 | chr15: 44717599-44717624 |
| B2M-51 | chr15: 44717604-44717629 |
| B2M-52 | chr15: 44717681-44717706 |
| B2M-53 | chr15: 44717682-44717707 |
| B2M-54 | chr15: 44717702-44717727 |
| B2M-55 | chr15: 44717764-44717789 |
| B2M-56 | chr15: 44717776-44717801 |
| B2M-57 | chr15: 44717786-44717811 |
| B2M-58 | chr15: 44717789-44717814 |
| B2M-59 | chr15: 44717790-44717815 |
| B2M-60 | chr15: 44717794-44717819 |
| B2M-61 | chr15: 44717805-44717830 |
| B2M-62 | chr15: 44717808-44717833 |
| B2M-63 | chr15: 44717809-44717834 |
| B2M-64 | chr15: 44717810-44717835 |
| B2M-65 | chr15: 44717846-44717871 |
| B2M-66 | chr15: 44717945-44717970 |
| B2M-67 | chr15: 44717946-44717971 |
| B2M-68 | chr15: 44717947-44717972 |
| B2M-69 | chr15: 44717948-44717973 |
| B2M-70 | chr15: 44717973-44717998 |
| B2M-71 | chr15: 44717981-44718006 |
| B2M-72 | chr15: 44718056-44718081 |
| B2M-73 | chr15: 44718061-44718086 |
| B2M-74 | chr15: 44718067-44718092 |
| B2M-75 | chr15: 44718076-44718101 |
| B2M-76 | chr15: 44717589-44717614 |
| B2M-77 | chr15: 44717620-44717645 |
| B2M-78 | chr15: 44717642-44717667 |
| B2M-79 | chr15: 44717771-44717796 |
| B2M-80 | chr15: 44717800-44717825 |
| B2M-81 | chr15: 44717859-44717884 |
| B2M-82 | chr15: 44717947-44717972 |
| B2M-83 | chr15: 44718119-44718144 |

10. The engineered cell of claim 8, wherein the genetic modification that inhibits expression of one or more MHC class I proteins is a genetic modification in an HLA-A sequence and optionally wherein the genetic modification is within the genomic coordinates chosen from chr6:29942854 to chr6:29942913 and chr6:29943518 to chr6: 29943619, optionally genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

11. The engineered cell of any one of claims 1-10, wherein the cell comprises a genetic modification, wherein the genetic modification inhibits expression of one or more MHC class II proteins.

12. The engineered cell of claim 11, wherein the genetic modification that inhibits expression of one or more MHC class II proteins is a genetic modification in a CIITA sequence, wherein the genetic modification is within the genomic coordinates selected from chr: 16:10902171-10923242, optionally, chr16:10902662-10923285, chr16:10906542-10923285, or chr16:10906542-10908121, optionally chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150; optionally chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-1090650; or optionally chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

13. The engineered cell of any one of claims 1-12, wherein the cell has reduced cell surface expression of PD1 protein, or wherein the cell has reduced cell surface expression of PD1 protein and the cell has reduced cell surface expression of TRAC protein or TRBC protein.

14. The engineered cell of any one of claims 1-13, comprising a genetic modification in a human 2B4/CD244 sequence, within genomic coordinates of chr1: 160830160-160862887.

15. The engineered cell of claim 14, wherein the genetic modification in 2B4/CD244 is within genomic coordinates selected from:

| 2B4 NO | Genomic Coordinates (hg38) |
|---|---|
| 2B4-1 | chr1: 160841611-160841631 |
| 2B4-2 | chr1: 160841865-160841885 |
| 2B4-3 | chr1: 160862624-160862644 |
| 2B4-4 | chr1: 160862671-160862691 |
| 2B4-5 | chr1: 160841622-160841642 |
| 2B4-6 | chr1: 160841819-160841839 |
| 2B4-7 | chr1: 160841823-160841843 |
| 2B4-8 | chr1: 160841717-160841737 |
| 2B4-9 | chr1: 160841859-160841879 |
| 2B4-10 | chr1: 160841806-160841826 |
| 2B4-11 | chr1: 160841834-160841854 |
| 2B4-12 | chr1: 160841780-160841800 |
| 2B4-13 | chr1: 160841713-160841733 |
| 2B4-14 | chr1: 160841631-160841651 |
| 2B4-15 | chr1: 160841704-160841724 |
| 2B4-16 | chr1: 160841584-160841604 |
| 2B4-17 | chr1: 160841679-160841699 |
| 2B4-18 | chr1: 160841874-160841894 |
| 2B4-19 | chr1: 160841750-160841770 |
| 2B4-20 | chr1: 160841577-160841597 |
| 2B4-21 | chr1: 160841459-160841479 |
| 2B4-22 | chr1: 160841466-160841486 |
| 2B4-23 | chr1: 160841461-160841481 |
| 2B4-24 | chr1: 160841460-160841480 |
| 2B4-25 | chr1: 160841360-160841380 |
| 2B4-26 | chr1: 160841304-160841324 |
| 2B4-27 | chr1: 160841195-160841215 |
| 2B4-28 | chr1: 160841305-160841325 |

or
the genomic coordinates selected from those targeted by 2B4-1 through 2B4-5: chr1: 160841611-160841631; chr1: 160841865-160841885; chr1: 160862624-160862644; chr1: 160862671-160862691; and chr1: 160841622-160841642; or
the genomic coordinates selected from those targeted by 2B4-1 and 2B4-2: chr1:160841611-160841631; and chr1:160841865-160841885; or
the genomic coordinates selected from those targeted by 2B4-3, 2B4-4, 2B4-10, and 2B4-17; chr1:160862624-160862644; chr1:160862671-160862691; chr1:160841806-160841826; and chr1:160841679-160841699.

16. The engineered cell of any one of claims 1-15, comprising a genetic modification in a human TIM3 sequence, within the genomic coordinates of chr5:157085832-157109044.

17. The engineered cell of claim 16, wherein the genetic modification in TIM3 is within genomic coordinates selected from:

| TIM 3 NO | Genomic Coordinates (hg38) |
|---|---|
| TIM3 - 1 | chr5: 157106867-157106887 |
| TIM3 - 2 | chr5: 157106862-157106882 |
| TIM3 - 3 | chr5: 157106803-157106823 |
| TIM3 - 4 | chr5: 157106850-157106870 |
| TIM3 - 5 | chr5: 157104726-157104746 |
| TIM3 - 6 | chr5: 157106668-157106688 |
| TIM3 - 7 | chr5: 157104681-157104701 |
| TIM3 - 8 | chr5: 157104681-157104701 |
| TIM3 - 9 | chr5: 157104680-157104700 |
| TIM3 - 10 | chr5: 157106676-157106696 |
| TIM3 - 11 | chr5: 157087271-157087291 |
| TIM3 - 12 | chr5: 157095432-157095452 |
| TIM3 - 13 | chr5: 157095361-157095381 |
| TIM3 - 14 | chr5: 157095360-157095380 |
| TIM3 - 15 | chr5: 157108945-157108965 |
| TIM3 - 18 | chr5: 157106751-157106771 |
| TIM3 - 19 | chr5: 157095419-157095439 |
| TIM3 - 22 | chr5: 157104679-157104699 |
| TIM3 - 23 | chr5: 157106824-157106844 |
| TIM3 - 26 | chr5: 157087117-157087137 |
| TIM3 - 29 | chr5: 157095379-157095399 |
| TIM3 - 32 | chr5: 157106864-157106884 |
| TIM3 - 42 | chr5: 157095405-157095425 |
| TIM3 - 44 | chr5: 157095404-157095424 |
| TIM3 - 56 | chr5: 157106888-157106908 |
| TIM3 - 58 | chr5: 157087126-157087146 |
| TIM3 - 59 | chr5: 157087253-157087273 |
| TIM3 - 62 | chr5: 157106889-157106909 |
| TIM3 - 63 | chr5: 157106935-157106955 |
| TIM3 - 66 | chr5: 157106641-157106661 |
| TIM3 - 69 | chr5: 157087084-157087104 |
| TIM3 - 75 | chr5: 157104663-157104683 |
| TIM3 - 82 | chr5: 157106875-157106895 |
| TIM3 - 86 | chr5: 157087184-157087204 |
| TIM3 - 87 | chr5: 157106936-157106956 |
| TIM3 - 88 | chr5: 157104696-157104716; |

or
the genomic coordinates selected from those targeted by TIM3-1 through TIM3-4, TIM3-6 through TIM3-15, TIM3-18, TIM3-19, TIM3-22, TIM3-29, TIM3-42, TIM3-44, TIM3-58, TIM3-62, TIM3-69, TIM3-82, TIM3-86, and TIM3-88: chr5:157106867-157106887; chr5:157106862-157106882; chr5:157106803-157106823; chr5:157106850-157106870; chr5:157106668-157106688; chr5:157104681-157104701; chr5:157104681-157104701; chr5:157104680-157104700; chr5:157106676-157106696; chr5:157087271-157087291; chr5:157095432-157095452; chr5:157095361-157095381; chr5:157095360-157095380; chr5:157108945-157108965; chr5:157106751-157106771; chr5:157095419-157095439; chr5:157104679-157104699; chr5:157095379-157095399; chr5:157095405-157095425; chr5:157095404-157095424; chr5:157087126-157087146; chr5:157106889-157106909; chr5:157087084-157087104; chr5:157106875-157106895; chr5:157087184-157087204; and chr5:157104696-157104716; or
the genomic coordinates selected from those targeted by TIM3-1 through TIM3-5, TIM3-7, TIM3-8, TIM3-12 through TIM3-15, TIM3-23, TIM3-26, TIM3-32, TIM3-56, TIM3-59, TIM3-63, TIM3-66, TIM3-75, and TIM3-87: chr5:157106867-157106887; chr5:157106862-157106882; chr5:157106803-157106823; chr5:157106850-157106870; chr5:157106668-157106688; chr5:157104681-157104701; chr5:157104681-157104701; chr5:157095432-157095452; chr5:157095361-157095381; chr5:157095360-157095380; chr5:157108945-157108965; chr5:157106824-157106844; chr5:157087117-157087137; chr5:157106864-157106884; chr5:157106888-157106908; chr5:157087253-157087273; chr5:157106935-157106955; chr5:157106641-157106661; chr5:157104663-157104683; and chr5:157106936-157106956; or
the genomic coordinates selected from those targeted by TIM3-2, TIM3-4, TIM3-15, TIM3-23, TIM3-56, TIM3-59, TIM3-63, TIM3-75, and TIM3-87; chr5:157106862-157106882; chr5:157106850-157106870; chr5:157108945-157108965; chr5:157106824-157106844; chr5:157106888-157106908; chr5:157087253-157087273; chr5:157106935-157106955; chr5:157104663-157104683; and chr5:157106936-157106956; or
the genomic coordinates selected from those targeted by TIM3-1 through TIM3-4: chr5:157106867-157106887; chr5:157106862-157106882; chr5:157106803-157106823; and chr5:157106850-157106870; or
the genomic coordinates selected from those targeted by TIM3-2, TIM3-4, and TIM3-15: chr5:157106862-157106882; chr5:157106850-157106870; and chr5:157108945-157108965; or
the genomic coordinates selected from those targeted by TIM3-2, TIM3-4, TIM3-15, TIM3-63, and TIM3-87; chr5:157106862-157106882; chr5:157106850-157106870; chr5:157108945-157108965; chr5:157106935-157106955; and chr5:157106936-157106956; or
the genomic coordinates selected from those targeted by TIM3-2 and TIM3-15: chr5:157106862-157106882; and chr5:157108945-157108965; or
the genomic coordinates selected from those targeted by TIM3-63 and TIM3-87: chr5:157106935-157106955; and chr5:157106936-157106956; or
the genomic coordinates selected from those targeted by TIM3-15: chr5:157108945-157108965.

18. The engineered cell of any one of claims 1-17, comprising a genetic modification in a human LAG3 sequence, within the genomic coordinates of chr12: 6772483-6778455.

19. The engineered cell of claim 18, wherein the genetic modification is within the genetic coordinates selected from:

| LAG 3 NO | Genomic Coordinates (hg38) |
|---|---|
| LAG3-1 | chr12: 6773938-6773958 |
| LAG3-2 | chr12: 6774678-6774698 |
| LAG3-3 | chr12: 6772894-6772914 |
| LAG3-4 | chr12: 6774816-6774836 |
| LAG3-5 | chr12: 6774742-6774762 |
| LAG3-6 | chr12: 6775380-6775400 |
| LAG3-7 | chr12: 6774727-6774747 |
| LAG3-8 | chr12: 6774732-6774752 |
| LAG3-9 | chr12: 6777435-6777455 |
| LAG3-10 | chr12: 6774771-6774791 |
| LAG3-11 | chr12: 6772909-6772929 |
| LAG3-12 | chr12: 6774735-6774755 |
| LAG3-13 | chr12: 6773783-6773803 |
| LAG3-14 | chr12: 6775292-6775312 |
| LAG3-15 | chr12: 6777433-6777453 |
| LAG3-16 | chr12: 6778268-6778288 |
| LAG3-17 | chr12: 6775444-6775464 |
| LAG3-24 | chr12: 6777783-6777803 |
| LAG3-26 | chr12: 6777784-6777804 |
| LAG3-41 | chr12: 6778252-6778272 |
| LAG3-59 | chr12: 6777325-6777345 |
| LAG3-83 | chr12: 6777329-6777349; |

or the genomic coordinates selected from those targeted by LAG3-1 through LAG3-15: chr12:6773938-6773958; chr12:6774678-6774698; chr12:6772894-6772914; chr12:6774816-6774836; chr12:6774742-6774762; chr12:6775380-6775400; chr12:6774727-6774747; chr12:6774732-6774752; chr12:6777435-6777455; chr12:6774771-6774791; chr12:6772909-6772929; chr12:6774735-6774755; chr12:6773783-6773803; chr12:6775292-6775312; and chr12:6777433-6777453; or
the genomic coordinates selected from those targeted by LAG3-1 through LAG3-11: chr12:6773938-6773958; chr12:6774678-6774698; chr12:6772894-6772914; chr12:6774816-6774836; chr12:6774742-6774762; chr12:6775380-6775400; chr12:6774727-6774747; chr12:6774732-6774752; chr12:6777435-6777455; chr12:6774771-6774791; and chr12:6772909-6772929; or
the genomic coordinates selected from those targeted by LAG3-1 through LAG3-4: chr12:6773938-6773958; chr12:6774678-6774698; chr12:6772894-6772914; and chr12:6774816-6774836; or
the genomic coordinates selected from those targeted by: LAG3-1, LAG3-4, LAG3-5, and LAG3-9: chr12:6773938-6773958; chr12:6774816-6774836; chr12:6774742-6774762; and chr12:6777435-6777455.

20. The engineered cell of any one of claims 1-19, wherein the genetic modification comprises an indel.

21. The engineered cell of any one of claims 1-20, wherein the genetic modification comprises an insertion of a heterologous coding sequence.

22. The engineered cell of any one of claims 1-21, wherein the genetic modification comprises a substitution, optionally wherein the substitution comprises a C to T substitution or an A to G substitution.

23. The engineered cell of any one of claims 1-22, wherein the genetic modification results in a change in the nucleic acid sequence that prevents translation of a full-length protein having an amino acid sequence of the full-length protein prior to genetic modification, optionally wherein the genetic modification results in a change in the nucleic acid sequence that results in a premature stop codon in a coding sequence of the full-length protein, or results in a change in splicing of a pre-mRNA from the genomic locus.

24. The engineered cell of any one of claims 1-23, wherein the cell comprises an exogenous nucleic acid encoding a targeting receptor that is expressed on the surface of the engineered cell, optionally wherein the targeting receptor is a CAR or TCR.

25. The engineered cell of any one of claims 1-24, wherein the engineered cell is a T cell.

26. A pharmaceutical composition comprising the engineered cell of any one of claims 1-25.

27. A population of cells comprising the engineered cell of any one of claims 1-25.

28. A method of administering the engineered cell, population of cells, or pharmaceutical composition of any one of claims 1-27 to a subject in need thereof.

29. A method of administering the engineered cell, population of cells, or pharmaceutical composition of any one of claims 1-27 to a subject as an adoptive cell transfer (ACT) therapy.

30. An engineered cell, population of cells, or pharmaceutical composition of any one of claims 1-27, for use as an ACT therapy.

31. A PD1 guide RNA that specifically hybridizes to a PD1 sequence, the guide RNA comprising a nucleotide sequence selected from:
1. a guide sequence comprising a nucleotide sequence selected from SEQ ID NOs: 1-88;
2. a guide sequence comprising a nucleotide sequence of at least 17, 18, 19, or 20 contiguous nucleotides of a nucleotide sequence selected from the sequence of SEQ ID NOs: 1-88;
3. a guide sequence comprising a nucleotide sequence at least 95% identical or at least 90% identical to a nucleotide sequence selected from SEQ ID Nos: 1-88;
4. a guide sequence comprising a nucleotide sequence selected from SEQ ID NOs: 5, 6, 8, 11, 12, 17, 22, 23, 24, 29, 36, 38, 41, 43, 56, 57 and 58;
5. a guide sequence comprising a nucleotide sequence selected from SEQ ID NOs: 5, 6, 8, 11, 12, 23, 24, 29, 36, 38, 43, and 57;
6. a guide sequence comprising a nucleotide sequence selected from SEQ ID NOs: 5, 11, 12, 22, 23, and 43;
7. a guide sequence comprising a nucleotide sequence selected from SEQ ID NOs: 6, 8, 23, and 29;
8. a guide sequence comprising a nucleotide sequence selected from SEQ ID NOs: 6 and 29;
9. a guide sequence comprising a nucleotide sequence selected from SEQ ID NOs: 6, 23, 29, 41, and 57;
10. a guide sequence comprising a nucleotide sequence selected from SEQ ID NOs: 6, 29, and 57; and
11. a guide sequence comprising a nucleotide sequence of SEQ ID NO: 43.

32. A PD1 guide RNA comprising a guide sequence that directs an RNA-guided DNA binding agent to a chromosomal location within the genomic coordinates selected from those targeted by SEQ ID NOs: 1-88; SEQ ID NOs: 5, 6, 8, 11, 12, 17, 22, 23, 24, 29, 36, 38, 41, 43, 56, 57 and 58; or SEQ ID NOs: 5, 6, 8, 11, 12, 22, 23, 24, 29, 36, 43, and 57; or SEQ ID NOs: 5, 11, 12, 22, 23, and 43; or SEQ ID NOs: 6, 8, 23, and 29; SEQ ID NOs: 6 and 29; or SEQ ID NOs: 6, 23, 29, 41, and 57; or SEQ ID NOs: 6, 29, and 57; or SEQ ID NO: 43.

33. The guide RNA of claim 31 or 32, wherein the guide RNA is a single guide RNA (sgRNA).

34. The guide RNA of claim 33, further comprising the nucleotide sequence of SEQ ID NO: 201 at the 3′ end to the guide sequence, wherein the guide RNA comprises a 5′ end modification or a 3′ end modification.

35. The guide RNA of claim 33, further comprising 5′ end modification or a 3′ end modification and a conserved portion of an gRNA comprising one or more of:
A. a shortened hairpin 1 region or a substituted and optionally shortened hairpin 1 region relative to SEQ ID NO: 201, wherein
1. at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9, and the hairpin 1 region optionally lacks
a. any one or two of H1-5 through H1-8,
b. one, two, or three of the following pairs of nucleotides: H1-1 and H1-12, H1-2 and H1-11, H1-3 and H1-10, and H1-4 and H1-9, or
c. 1-8 nucleotides of hairpin 1 region; or
2. the shortened hairpin 1 region lacks 4-8 nucleotides, preferably 4-6 nucleotides; and
a. one or more of positions H1-1, H1-2, or H1-3 is deleted or substituted relative to SEQ ID NO: 201; or
b. one or more of positions H1-6 through H1-10 is substituted relative to SEQ ID NO: 201; or
1. the shortened hairpin 1 region lacks 5-10 nucleotides, preferably 5-6 nucleotides, and one or more of positions N18, H1-12, or n is substituted relative to SEQ ID NO: 201; or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 201; or
C. a substitution relative to SEQ ID NO: 201 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D, an upper stem region, wherein the upper stem modification comprises a modification to any one or more of US1-US12 in the upper stem region.

36. The guide RNA of claim 33 or 34, wherein the guide RNA is modified according to the pattern mN*mN*mN*NNNNNNNNNNNNNNNNNGUUUUAGAmGmCmUmAmGmAmA mAmUmAmGmCAAGUUAAAAUAAGGCUAGUCCGUUAUCAmAmCmUmUm GmAmAmAmAmAmGmUmGmGmCmAmCmCmGmAmGmUmCmGmGmUmGm CmU*mU*mU*mU (SEQ ID NO: 300), where “N” may be any natural or non-natural nucleotide, m is a 2′-O-methyl modified nucleotide, and * is a phosphorothioate linkage between nucleotide residues; and wherein the N's are collectively the nucleotide sequence of a guide sequence of any preceding claim, optionally wherein each N is independently any natural or non-natural nucleotide and the guide sequence targets Cas9 to the PD1 gene.

37. The guide RNA of any one of claims 33-36, wherein the guide RNA comprises a modification.

38. The guide RNA of claim 37, wherein the modification comprises (i) a 2′-O-methyl (2′-O-Me) modified nucleotide or a 2′-F modified nucleotide, (ii) a 2′-F modified nucleotide, (iii) a phosphorothioate (PS) bond between nucleotides, (iv) a modification at one or more of the first five nucleotides at the 5′ end of the guide RNA, (v) a modification at one or more of the last five nucleotides at the 3′ end of the guide RNA, (vi) a PS bond between each of the first four nucleotides of the guide RNA, (vii) a PS bond between each of the last four nucleotides of the guide RNA, (viii) a 2′-O-Me modified nucleotide at each of the first three nucleotides at the 5′ end of the guide RNA, (ix) a 2′-O-Me modified nucleotide at each of the last three nucleotides at the 3′ end of the guide RNA, or combinations of one or more of (i)-(ix).

39. A composition comprising a guide RNA of any one of claims 31-38 and an RNA guided DNA binding agent wherein the RNA guided DNA binding agent is a polypeptide RNA guided DNA binding agent or a nucleic acid encoding an RNA guided DNA binding agent polypeptide, optionally wherein the RNA guided DNA-binding agent is a Cas9 nuclease.

40. The guide RNA of any one of claims 31-38 or the composition of claim 39, wherein the composition further comprises a pharmaceutically acceptable excipient.

41. The guide RNA or composition of any one of claims 31-40, wherein the guide RNA is associated with a lipid nanoparticle (LNP).

42. A method of making a genetic modification in a PD1 sequence within a cell, comprising contacting the cell with the guide RNA or composition of any one of claims 31-41.

43. The method of claim 42, further comprising making a genetic modification in a TCR sequence to inhibit expression of a TCR gene.

44. A method of preparing a population of cells for immunotherapy comprising:
a. making a genetic modification in a PD1 sequence in the cells in the population with a PD1 guide RNA or composition of any one of claims 31-41;
b. making a genetic modification in a TCR sequence in the cells of the population to reduce expression of the TCR protein on the surface of the cells in the population;
c. expanding the population of cells in culture.

45. A population of cells made by the method of any one of claims 42-44.

46. The population of cells of claim 45, wherein the population of cells is altered ex vivo.

47. A method of administering the population of cells of claim 45 or 46 to a subject in need thereof.

48. A method of administering the population of cells of claim 45 or 46 to a subject as an adoptive cell transfer (ACT) therapy.

49. A population of cells of claim 45 or 46 for use as an ACT therapy.

50. A population of cells comprising a genetic modification of a PD1 gene, wherein at least 40%, 45%, 50%, 55%, 60%, 65%, preferably at least 70%, 75%, 80%, 85%, 90%, or 95% of cells in the population comprise a modification selected from an insertion, a deletion, and a substitution in the endogenous PD1 sequence.

51. The population of cells of claim 50, wherein expression of PD1 is decreased by at least 40%, 45%, 50%, 55%, 60%, 65%, preferably at least 70%, 75%, 80%, 85%, 90%, 95%, or to below the limit of detection of the assay as compared to a suitable control, e.g., wherein the PD1 gene has not been modified.

52. The population of cells of claim 50 or 51, wherein at least 70%, at least 80%, at least 90%, or at least 95% of cells in the population comprise a modification selected from an insertion, a deletion, and a substitution in the endogenous PD1 sequence.
